SEQUENCE LISTING (58 sequences)

SEQ ID NO: 1 - protein, 4 aa, synthetic construct
KERE

SEQ ID NO: 2 - protein, 4 aa, synthetic construct
KQRE

SEQ ID NO: 3 - protein, 4 aa, synthetic construct
GLEW

SEQ ID NO: 4 - protein, 5 aa, synthetic construct
KEREL

SEQ ID NO: 5 - protein, 5 aa, synthetic construct
KEREF

SEQ ID NO: 6 - protein, 5 aa, synthetic construct
KQREL

SEQ ID NO: 7 - protein, 5 aa, synthetic construct
KQREF

SEQ ID NO: 8 - protein, 5 aa, synthetic construct
KEREG

SEQ ID NO: 9 - protein, 5 aa, synthetic construct
KQREW

SEQ ID NO: 10 - protein, 5 aa, synthetic construct
KQREG

SEQ ID NO: 11 - protein, 4 aa, synthetic construct
TERE

SEQ ID NO: 12 - protein, 5 aa, synthetic construct
TEREL

SEQ ID NO: 13 - protein, 4 aa, synthetic construct
TQRE

SEQ ID NO: 14 - protein, 5 aa, synthetic construct
TQREL

SEQ ID NO: 15 - protein, 4 aa, synthetic construct
KECE

SEQ ID NO: 16 - protein, 5 aa, synthetic construct
KECEL

SEQ ID NO: 17 - protein, 5 aa, synthetic construct
KECER

SEQ ID NO: 18 - protein, 4 aa, synthetic construct
KQCE

SEQ ID NO: 19 - protein, 5 aa, synthetic construct
KQCEL

SEQ ID NO: 20 - protein, 4 aa, synthetic construct
RERE

SEQ ID NO: 21 - protein, 5 aa, synthetic construct
REREG

SEQ ID NO: 22 - protein, 4 aa, synthetic construct
RQRE

SEQ ID NO: 23 - protein, 5 aa, synthetic construct
RQREL

SEQ ID NO: 24 - protein, 5 aa, synthetic construct
RQREF

SEQ ID NO: 25 - protein, 5 aa, synthetic construct
RQREW

SEQ ID NO: 26 - protein, 4 aa, synthetic construct
QERE

SEQ ID NO: 27 - protein, 5 aa, synthetic construct
QEREG

SEQ ID NO: 28 - protein, 4 aa, synthetic construct
QQRE

SEQ ID NO: 29 - protein, 5 aa, synthetic construct
QQREW

SEQ ID NO: 30 - protein, 5 aa, synthetic construct
QQREL

SEQ ID NO: 31 - protein, 5 aa, synthetic construct
QQREF

SEQ ID NO: 32 - protein, 4 aa, synthetic construct
KGRE

SEQ ID NO: 33 - protein, 5 aa, synthetic construct
KGREG

SEQ ID NO: 34 - protein, 4 aa, synthetic construct
KDRE

SEQ ID NO: 35 - protein, 5 aa, synthetic construct
KDREV

SEQ ID NO: 36 - protein, 5 aa, synthetic construct
DECKL

SEQ ID NO: 37 - protein, 5 aa, synthetic construct
NVCEL

SEQ ID NO: 38 - protein, 4 aa, synthetic construct
GVEW

SEQ ID NO: 39 - protein, 4 aa, synthetic construct
EPEW

SEQ ID NO: 40 - protein, 4 aa, synthetic construct
GLER

SEQ ID NO: 41 - protein, 4 aa, synthetic construct
DQEW

SEQ ID NO: 42 - protein, 4 aa, synthetic construct
DLEW

SEQ ID NO: 43 - protein, 4 aa, synthetic construct
GIEW

SEQ ID NO: 44 - protein, 4 aa, synthetic construct
ELEW

SEQ ID NO: 45 - protein, 4 aa, synthetic construct
GPEW

SEQ ID NO: 46 - protein, 4 aa, synthetic construct
EWLP

SEQ ID NO: 47 - protein, 4 aa, synthetic construct
GPER

SEQ ID NO: 48 - protein, 4 aa, synthetic construct
GLER

SEQ ID NO: 49 - protein, 4 aa, synthetic construct
ELEW

SEQ ID NO: 50 - protein, 231 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 330 aa, Homo sapiens [sequence omitted]

SEQ ID NO: 52 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 56 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 57 - protein, 229 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 58 - protein, 228 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

(19) United States
(12) Patent Application Publication
(10) Pub. No.: US 2025/0154262 A1
(43) Pub. Date: May 15, 2025

(54) Fc DOMAIN VARIANTS WITH ENHANCED Fc RECEPTOR BINDING

(71) Applicant: ABLYNX N.V., Zwijnaarde (BE)

(72) Inventors: Julie A. JAWORSKI, Bolton, MA (US); Sagar V. KATHURIA, Harvard, MA (US); Brian MACKNESS, Lowell, MA (US); Sunghae PARK, Waban, MA (US); Victor Zhao SUN, Cambridge, MA (US); Qun ZHOU, Ashland, MA (US)

(21) Appl. No.: 18/925,338

(22) Filed: Oct. 24, 2024

Related U.S. Application Data
(60) Provisional application No. 63/638,212, filed on Apr. 24, 2024.
(60) Provisional application No. 63/593,011, filed on Oct. 25, 2023.

(51) Int. Cl. C07K 16/28

(52) U.S. Cl. CPC C07K 16/283; C07K 2317/52; C07K 2317/72; C07K 2317/92; C07K 2317/94

(57) ABSTRACT

The present disclosure provides Fc domain variant polypeptides, including effector-competent Fc domain variants. The present disclosure also provides Fc domain variant polypeptides comprising at least one substitution or at least two substitutions at amino acid positions 251, 267, 268, 298, 314, 330, 339, 373, and 376, according to EU numbering, as compared to a Fc domain parent polypeptide. The present disclosure also provides nucleic acids encoding Fc domain variant polypeptides and host cells for making Fc domain variant polypeptides. Methods for increasing the yield of Fc domain variant polypeptides, and methods of using Fc domain variant polypeptides to treat disease, are also provided.

### RELATED APPLICATIONS

This application claims the benefit of U.S. Patent Application Serial Nos. 63/638,212, filed Apr. 24, 2024, and 63/593,011, filed Oct. 25, 2023, the entire disclosures of which are hereby incorporated herein by reference.

### SEQUENCE LISTING

The instant application contains a Sequence Listing which has been submitted electronically in XML format and is hereby incorporated by reference in its entirety. Said XMLfile, created on Oct. 24, 2024, is named 758603_SA9-318CIP_ST26.xml and is 53,245 bytes in size.

### BACKGROUND

The specific engagement between the fragment crystallizable (Fc) region of an antibody and a Fc gamma receptor (FcγR) is the initial step in effector functions such as antibody-dependent cell-mediated cytotoxicity (ADCC). In humans, activating FcγRIIIa is expressed on the surface of natural killer cells. FcγRIIIa is a low-affinity receptor, and activation of cells results from crosslinking of these surface receptors upon engagement of clustered Fc regions in antibody-antigen immune complexes. The Fc region also interacts with the neonatal Fc receptor (FcRn). This interaction has been shown to extend the half-life of IgG by reducing lysosomal degradation in endothelial cells.

Efforts have been made to enhance ADCC for increasing the efficacy in the treatment against diseases via Fc engineering to identify Fc domain variants that enhance affinity to the Fc receptors and to therefore enhance ADCC activity and/or serum half-life. Despite these efforts, there remains a need to create new Fc variant domain polypeptides with enhanced efficacy and improved manufacturability for use in treating various diseases.

### SUMMARY

The present disclosure Fc domain variant polypeptides comprising at least one substitution or at least two substitutions at amino acid positions 251, 267, 268, 298, 314, 330, 339, 373, and 376, according to EU numbering, as compared to a Fc domain parent polypeptide. The Fc domain variant polypeptides have increased binding to a Fc receptor relative to the Fc domain parent polypeptide as well as high protein expression and protein purity profiles.

In one aspect, a Fc domain variant polypeptide, wherein the Fc domain variant polypeptide comprises at least two substitutions at amino acid positions 251, 267, 268, 298, 314, 330, 339, 373, and 376, according to EU numbering, as compared to a Fc domain parent polypeptide. In another aspect, a Fc domain variant polypeptide has increased binding to a Fc receptor relative to the Fc domain parent polypeptide. In another aspect, at least two amino acid substitutions are selected from: (i) an alanine (A), a histidine (H), an isoleucine (I), a phenylalanine (F), a glutamine (Q), or a tryptophan (W) at amino acid position 251, (ii) an alanine (A) or an aspartic acid (D) at amino acid position 267, (iii) an aspartic acid (D) or a glutamic acid (E) at amino acid position 268, (iv) an alanine (A) at amino acid position 298, (v) a glycine (G), a lysine (K), an asparagine (N), a methionine (M), a serine (S), a threonine (T), a valine (V), a glutamic acid (E), or a tryptophan (W) at amino acid position 314, (vi) a phenylalanine (F), a methionine (M), or a tyrosine (Y) at amino acid position 330, (vii) a threonine (T) at amino acid position 339, (viii) a tryptophan (W) at amino acid position 373, or (ix) a valine (V) at amino acid position 376, according to EU numbering. In one aspect, a Fc domain parent polypeptide is a wild-type Fc domain.

In one aspect, a Fc domain variant comprises an aspartic acid (D) at amino acid position 268, a glutamine (Q) at amino acid position 251, a tryptophan (W) at amino acid position 373, and an alanine (A) at amino acid position 298, according to EU numbering.

In one aspect, a Fc domain variant comprises an aspartic acid (D) at amino acid position 268, a glutamine (Q) at amino acid position 251, and an alanine (A) at amino acid position 298, according to EU numbering.

In one aspect, a Fc domain variant comprises an aspartic acid (D) at amino acid position 268, an isoleucine (I) at amino acid position 251, a phenylalanine (F) at amino acid position 330, and an alanine (A) at amino acid position 298, according to EU numbering.

In one aspect, a Fc domain variant comprises an aspartic acid (D) at amino acid position 268, a threonine (T) at amino acid position 339, and an alanine (A) at amino acid position 298, according to EU numbering.

In one aspect, a Fc domain variant comprises a glutamic acid (E) at amino acid position 268, a valine (V) at amino acid position 376, an alanine (A) at amino acid position 251, and a methionine (M) at amino acid position 330, according to EU numbering.

In one aspect, a Fc domain variant comprises an aspartic acid (D) at amino acid position 268, an aspartic acid (D) at amino acid position 267, an isoleucine (I) at amino acid position 251, and a threonine (T) at amino acid position 339, according to EU numbering.

In one aspect, a Fc domain variant comprises an aspartic acid (D) at amino acid position 268, a tyrosine (Y) at amino acid position 330, a threonine (T) at amino acid position 339, a tryptophan (W) at amino acid position 373, according to EU numbering.

In one aspect, a Fc domain variant comprises an aspartic acid (D) at amino acid position 268, a glutamine (Q) at amino acid position 251, a tryptophan (W) at amino acid position 373, according to EU numbering.

In one aspect, a Fc domain variant comprises an aspartic acid (D) at amino acid position 268 and a threonine (T) at amino acid position 314, according to EU numbering.

In one aspect, a Fc domain variant comprises an aspartic acid (D) at amino acid position 268, a phenylalanine (F) at amino acid position 251, and a valine (V) at amino acid position 314, according to EU numbering.

In one aspect, a Fc domain variant comprises an aspartic acid (D) at amino acid position 268, a tyrosine (Y) at amino acid position 330, a valine (V) at amino acid position 376, according to EU numbering.

In one aspect, a Fc domain variant polypeptide comprises an alanine (A) at amino acid position 298, a tyrosine (Y) at amino acid position 330, a threonine (T) at amino acid position 339, and a tryptophan (W) at amino acid position 373, according to EU numbering.

In one aspect, a Fc domain variant comprises an aspartic acid (D) at amino acid position 268 and an alanine (A) at amino acid position 251, according to EU numbering.

In one aspect, a Fc domain variant comprises an aspartic acid (D) at amino acid position 267, a tyrosine (Y) at amino acid position 330, a threonine (T) at amino acid position 339, according to EU numbering.

In one aspect, a Fc domain variant comprises an aspartic acid (D) at amino acid position 267, a tyrosine (Y) at amino acid position 330, a tryptophan (W) at amino acid position 373, according to EU numbering.

In one aspect, a Fc domain variant comprises a glutamic acid (E) at amino acid position 268, a histidine (H) at amino acid position 251, a phenylalanine (F) at amino acid position 330, according to EU numbering.

In one aspect, a Fc domain variant comprises an aspartic acid (D) at amino acid position 268, a tryptophan (W) at amino acid position 373, an alanine (A) at amino acid position 298, according to EU numbering.

In one aspect, a Fc domain variant comprises an aspartic acid (D) at amino acid position 268 and an alanine (A) at amino acid position 298, according to EU numbering.

In one aspect, a Fc domain variant comprises an aspartic acid (D) at amino acid position 268, a phenylalanine (F) at amino acid position 330, and an alanine (A) at amino acid position 298, according to EU numbering.

In one aspect, a Fc domain variant comprises a glutamic acid (E) at amino acid position 268, a valine (V) at amino acid position 314, a methionine (M) at amino acid position 330, according to EU numbering.

In one aspect, a Fc domain variant comprises an aspartic acid (D) at amino acid position 268, an aspartic acid (D) at amino acid position 267, a threonine (T) at amino acid position 339, according to EU numbering.

In one aspect, a Fc domain variant comprises an aspartic acid (D) at amino acid position 268 and a tryptophan (W) at amino acid position 373, according to EU numbering.

In one aspect, a Fc domain variant comprises an aspartic acid (D) at amino acid position 268 and a valine (V) at amino acid position 314, according to EU numbering.

In one aspect, a Fc domain variant comprises a glutamic acid (E) at amino acid position 268 and a phenylalanine (F) at amino acid position 330, according to EU numbering.

In one aspect, a Fc domain variant comprises an aspartic acid (D) at amino acid position 268, a glutamine (Q) at amino acid position 251, and a glutamic acid (E) at amino acid position 314, according to EU numbering.

In one aspect, a Fc domain variant comprises an aspartic acid (D) at amino acid position 268, a glutamine (Q) at amino acid position 251, and a methionine (M) at amino acid position 314, and an alanine (A) at amino acid position 298, according to EU numbering.

In one aspect, a Fc domain variant comprises an aspartic acid (D) at amino acid position 268, a tryptophan (W) at amino acid position 251, and a serine (S) at amino acid position 314, according to EU numbering.

In one aspect, a Fc domain variant comprises an aspartic acid (D) at amino acid position 268, a tryptophan (W) at amino acid position 251, and an asparagine (N) at amino acid position 314 according to EU numbering.

In one aspect, a Fc domain variant comprises an aspartic acid (D) at amino acid position 268, a phenylalanine (F) at amino acid position 251, a methionine (M) at amino acid position 314, and an alanine (A) at amino acid position 298, according to EU numbering.

In one aspect, a Fc domain variant comprises an aspartic acid (D) at amino acid position 268, a tryptophan (W) at amino acid position 314, a tryptophan (W) at amino acid position 373, and a methionine (M) at amino acid position 330, according to EU numbering.

In one aspect, a Fc domain variant comprises a glutamic acid (E) at amino acid position 268, an isoleucine (I) at amino acid position 251, a threonine (T) at amino acid position 314, and a valine (V) at amino acid position 376, according to EU numbering.

In one aspect, a Fc domain variant comprises an aspartic acid (D) at amino acid position 268, a glutamine (Q) at amino acid position 251, and a glycine (G) at amino acid position 314, according to EU numbering.

In one aspect, a Fc domain variant comprises an aspartic acid (D) at amino acid position 268, a tryptophan (W) at amino acid position 251, and a glycine (G) at amino acid position 314, and an alanine (A) at amino acid position 298, according to EU numbering.

In one aspect, a Fc domain variant comprises an aspartic acid (D) at amino acid position 268, a tyrosine (Y) at amino acid position 330, and a lysine (K) at amino acid position 314, according to EU numbering.

In one aspect, a Fc domain variant comprises an aspartic acid (D) at amino acid position 268, a tryptophan (W) at amino acid position 251, a threonine (T) at amino acid position 314, and an alanine (A) at amino acid position 298, according to EU numbering.

In one aspect, a Fc domain variant comprises a glutamic acid (E) at amino acid position 268, a valine (V) at amino acid position 314, an alanine (A) at amino acid position 251, and a methionine (M) at amino acid position 330, according to EU numbering.

In one aspect, a Fc domain variant comprises an alanine (A) at amino acid position 298, a tyrosine (Y) at amino acid position 330, and a threonine (T) at amino acid position 339, according to EU numbering.

In one aspect, a Fc domain variant comprises an aspartic acid (D) at amino acid position 268, a phenylalanine (F) at amino acid position 251, and a lysine (K) at amino acid position 314 according to EU numbering.

In one aspect, a Fc domain variant comprises an aspartic acid (D) at amino acid position 268, a phenylalanine (F) at amino acid position 251, and a glycine (G) at amino acid position 314, according to EU numbering.

In one aspect, a Fc domain variant comprises an aspartic acid (D) at amino acid position 268, a tyrosine (Y) at amino acid position 330, a tryptophan (W) at amino acid position 251, and a valine (V) at amino acid position 376, according to EU numbering.

In one aspect, a Fc domain variant comprises a tyrosine (Y) at amino acid position 330, an alanine (A) at amino acid position 267, a valine (V) at amino acid position 376, and an alanine (A) at amino acid position 298, according to EU numbering.

In one aspect, a Fc domain variant comprises an aspartic acid (D) at amino acid position 268, a phenylalanine (F) at amino acid position 251, and a threonine (T) at amino acid position 314, according to EU numbering.

In one aspect, a Fc domain variant comprises an aspartic acid (D) at amino acid position 268, a tryptophan (W) at amino acid position 251, a methionine (M) at amino acid position 314, and an alanine (A) at amino acid position 298, according to EU numbering.

In one aspect, a Fc domain variant comprises an aspartic acid (D) at amino acid position 268, a tyrosine (Y) at amino acid position 330, and a tryptophan (W) at amino acid position 373, according to EU numbering.

In one aspect, a Fc domain variant comprises an aspartic acid (D) at amino acid position 268 and a glutamic acid (E) at amino acid position 314, according to EU numbering.

In one aspect, a Fc domain variant comprises an aspartic acid (D) at amino acid position 268, a methionine (M) at amino acid position 314, and an alanine (A) at amino acid position 298, according to EU numbering.

In one aspect, a Fc domain variant comprises an aspartic acid (D) at amino acid position 268, a tryptophan (W) at amino acid position 373, and an alanine (A) at amino acid position 298, according to EU numbering.

In one aspect, a Fc domain variant comprises an aspartic acid (D) at amino acid position 268 and a serine (S) at amino acid position 314, according to EU numbering.

In one aspect, a Fc domain variant comprises an aspartic acid (D) at amino acid position 268 and an asparagine (N) at amino acid position 314, according to EU numbering.

In one aspect, a Fc domain variant comprises a glutamic acid (E) at amino acid position 268, a threonine (T) at amino acid position 314, and a valine (V) at amino acid position 376, according to EU numbering.

In one aspect, a Fc domain variant comprises an aspartic acid (D) at amino acid position 268 and a glycine (G) at amino acid position 314, according to EU numbering.

In one aspect, a Fc domain variant comprises an aspartic acid (D) at amino acid position 268, a threonine (T) at amino acid position 314, and an alanine (A) at amino acid position 298, according to EU numbering.

In one aspect, a Fc domain variant comprises a glutamic acid (E) at amino acid position 268, a valine (V) at amino acid position 314, and a methionine (M) at amino acid position 330, according to EU numbering.

In one aspect, a Fc domain variant comprises an aspartic acid (D) at amino acid position 268 and a lysine (K) at amino acid position 314, according to EU numbering.

In one aspect, a Fc domain variant comprises an aspartic acid (D) at amino acid position 268, a tyrosine (Y) at amino acid position 330, and a valine (V) at amino acid position 376, according to EU numbering.

In one aspect, a Fc domain variant comprises an aspartic acid (D) at amino acid position 268, a glutamine (Q) at amino acid position 251, a methionine (M) at amino acid position 314, and an alanine (A) at amino acid position 298, according to EU numbering.

In one aspect, a Fc domain variant comprises an aspartic acid (D) at amino acid position 268, an aspartic acid (D) at amino acid position 267, and a threonine (T) at amino acid position 339, according to EU numbering.

In one aspect, a Fc domain variant comprises an aspartic acid (D) at amino acid position 268 and a threonine (T) at position 314, according to EU numbering.

In one aspect, a Fc domain variant comprises an aspartic acid (D) at amino acid position 268 and a glutamic acid (E) at position 314, according to EU numbering.

In one aspect, a Fc domain variant comprises an aspartic acid (D) at amino acid position 268 and a glycine (G) at position 314, according to EU numbering.

In one aspect, a Fc domain variant comprises an aspartic acid (D) at amino acid position 268 and a serine (S) at position 314, according to EU numbering.

In one aspect, a Fc domain variant comprises an aspartic acid (D) at amino acid position 268, a tyrosine (Y) at amino acid position 330, a valine (V) at position 376, according to EU numbering.

In one aspect, a Fc domain variant comprises a glutamic acid (E) at amino acid position 268, a threonine (T) at amino acid position 314, a valine (V) at position 376, according to EU numbering.

In one aspect, a Fc domain variant comprises an aspartic acid (E) at amino acid position 268, a valine (V) at amino acid position 314, and a methionine (M) at amino acid position 330, according to EU numbering.

In one aspect, a Fc domain variant of the disclosure comprises a Fc domain variant of mammalian origin. In another aspect, a Fc domain variant is of human origin.

In another aspect, a Fc domain variant is derived from an immunoglobulin class selected from a group consisting of: IgM, IgG, IgD, IgA, and IgE. In another aspect, a Fc domain variant is derived from an IgG Fc domain. In another aspect, a Fc domain variant is derived from a IgG1 Fc domain or an IgG4 Fc domain.

In one aspect, a Fc domain variant of the disclosure further comprises an aspartic acid (D) at amino acid position 239.

In one aspect, a Fc domain variant of the disclosure further comprises a glutamic acid (E) at amino acid position 332.

In one aspect, a Fc domain variant of the disclosure further comprises an aspartic acid (D) at amino acid position 239 and a glutamic acid (E) at amino acid position 332.

In one aspect, a Fc domain variant of the disclosure is about 20%, 30%, 40%, 50%, 60%, 70%, 90% or more afucosylated.

In one aspect, a Fc domain variant of the disclosure further comprises a cysteine (C) at amino acid position 292 and a cysteine (C) at amino acid position 302.

In one aspect, a Fc domain variant of the disclosure further comprises at least one N-glycan. In another aspect, the N-glycan comprises a mannose and/or a GlcNAc. In another aspect, a N-glycan is an oligomannose-type. In another aspect, an oligomannose-type N-glycan comprise an oligosaccharide selected from the group consisting of Man₉(GlcNAc)₂, Man₈(GlcNAc)₂, Man₇(GlcNAc)₂, Man₆(GlcNAc)₂, and Man₅(GlcNAc)₂. In another aspect, a Fc domain variant polypeptide comprises 20%, 30%, 40%, 50%, 60%, 70%, 90% or more Man₅₋₉(GlcNAc)₂ glycans by molar ratio relative to all N-glycans. In another aspect, a Fc domain variant polypeptide comprises greater than 70%, 75%, 80%, 85%, 90%, or 95% Man₅₋₉(GlcNAc)₂ N-glycans by molar ratio relative to all N-glycans. In another aspect, a Fc domain variant polypeptide comprises Man₈ and Man₉ together as the major species of Man₅₋₉(GlcNAc)₂ N-glycans. In another aspect, a Fc domain variant polypeptide comprises at least 97% Man₅₋₉(GlcNAc)₂ N-glycans by molar ratio relative to all N-glycans. In another aspect, a Fc domain variant polypeptide comprises less than 30%, 20%, 10%, 5%, 1% of Man₅₋₉(GlcNAc)₂ glycans by molar ratio relative to all N-glycans, or substantially no Man₅₋₉(GlcNAc)₂ glycans by molar ratio relative to all N-glycans.

In one aspect, a Fc domain variant of the disclosure further comprises a cysteine (C) at amino acid position 292 and a cysteine (C) at amino acid position 302.

In one aspect, a Fc domain variant of the disclosure is produced by culturing cells that express the Fc domain variant polypeptide in the presence of a mannosidase inhibitor. In another aspect, the mannosidase inhibitor is kifunensine. In another aspect, the concentration of kifunensine is from about 60 ng/mL to about 2500 ng/mL. In another aspect, the concentration of kifunensine is about 2000 ng/mL.

In one aspect, a Fc domain variant of the disclosure comprises at least one binding site for selectively binding to a target antigen of interest and the Fc domain variant polypeptide. In another aspect, the binding polypeptide is a multispecific binding polypeptide. In another aspect, the binding polypeptide is an antibody or an antigen fragment thereof. In another aspect, the binding polypeptide is a multispecific antibody. In another aspect, the binding polypeptide comprises a VHH. In another aspect, the binding polypeptide comprises at least one antigen binding fragment selected from a group consisting of: a variable fragment (Fv), a Fab, a Fab′, a F(ab′)₂, a minibody, a diabody, a triabody, a tetrabody, a tandem di-scFv, a tandem tri-scFv, an immunoglobulin single variable domain (ISV).

In one aspect, a Fc domain variant of the disclosure comprises a human Fc receptor. In another aspect, the Fc receptor is a Fcγ receptor (FcγR). In another aspect, the FcγR is a FcγRI, FcγRIIA, FcγRIIB, FcγRIIIA, or a FcγRIIIB. In another aspect, the FcγRIIIa is hFcγRIIIa with a valine at amino acid position 158, according to EU numbering (FcγRIIIaᵥ₁₅₈). In another aspect, the FcγRIIIa is hFcγRIIIa with a phenylalanine at amino acid position 158, according to EU numbering (FcγRIIIa_{F158}). In another aspect, the Fc receptor is a neonatal Fc receptor (FcRn).

In one aspect, a Fc domain variant of the disclosure comprises a Fc domain parent polypeptide is a wild-type Fc domain. In another aspect, the Fc domain parent polypeptide comprises a modified Fc domain. In another aspect, the Fc domain parent polypeptide comprises at least one modified glycan. In another aspect, the modified glycan is a bis mannose 6 phosphate (bisM6P), a disaccharide mannose 6 phosphate, or a mannose 6 phosphate monosaccharide. In another aspect, the modified glycan is an oligomannose-type N-glycan comprising an oligosaccharide selected from the group consisting of: a Man₉(GlcNAc)₂, a Man₈(GlcNAc)₂, a Man₇(GlcNAc)₂, a Man₆(GlcNAc)₂, or a Man₅(GlcNAc)₂.

In one aspect, a Fc domain variant of the disclosure has about a 1.5-fold to a 20-fold increased binding affinity to a Fc receptor relative to the Fc domain parent polypeptide.

In one aspect, a Fc domain variant of the disclosure comprising a Fc domain variant has a production titer ranging from about 5 to about 500 milligrams per liter (mg/L).

In one aspect, a Fc domain variant of the disclosure comprising a Fc domain variant has at least 60, 70, 80, 90, or 100% purity when measured by size exclusion chromatography (SEC).

In one aspect, a pharmaceutical composition comprises the Fc domain variant polypeptide and a pharmaceutically acceptable carrier or diluent.

In one aspect, a nucleic acid molecule encodes the Fc domain variant polypeptide of the disclosure. In another aspect, a vector comprises the nucleic acid molecule encoding the Fc domain variant polypeptide. In another aspect, a producing cell expresses the nucleic acid molecule.

In order to provide a clear understanding of the specification and claims, the following definitions are provided.

### BRIEF DESCRIPTION OF THE DRAWINGS

The foregoing and other features and advantages of the present application will be more fully understood from the following detailed description of illustrative aspects taken in conjunction with the accompanying drawings.

FIG. 1 is a bar graph displaying the protein production for Fc domain variant polypeptides in milligram (mg) quantities.

FIG. 2 is a bar graph displaying the percent protein purity for Fc domain variant polypeptides as measured by size exclusion chromatography (SEC).

FIG. 3 is sensorgram data collected from the four Fc domain polypeptides used as controls. The four controls were: (1) a commercially available antibody with a wild-type IgG domain (adalimumab; left top panel); (2) the same antibody as (1) with a Fc-domain mutation that facilitated glycoengineering which abolished effector functions including hFcγRIIIa binding (an antibody with a S298N/T299A/Y300S Fc domain mutation; right top panel); (3) an antibody with a Fc-domain mutation that enhanced hFcγRIIIa binding (bottom left panel); and (4) the antibody in (3) with glycoengineering that further enhanced hFcγRIIIa binding.

FIG. 4 is sensorgram data measuring hFcγRIIIa binding affinity collected from 80 Fc domain variant polypeptides and the respective controls.

FIG. 5 is sensorgram data measuring hFcγRIIIa binding affinity collected from 45 Fc domain variant polypeptides and the respective controls.

FIG. 6 is an isoaffinity plot of 125 Fc domain variant polypeptides relative to controls (Ka, 1/Ms, y-axis; Kd, 1/s, x-axis).

FIG. 7 is a table binning the binding affinities of 125 Fc domain variant polypeptides relative to the binding affinity of wild-type IgG Fc domain (adalimumab).

FIGS. 8A-B. FIG. 8A shows a table of the 16 Fc domain variant polypeptides candidates with favorable protein expression, protein purity, and hFcγRIIIa binding kinetic profiles. FIG. 8B is a legend of the criteria applied to FIG. 8A.

FIG. 9 are tables of the correspondence between the IMGT unique numbering for C-DOMAIN, the IMGT exon numbering, the Eu and Kabat numberings for Human IGHG. The tables are for the CH2 and CH3 domains and were extracted from https://www.imgt.org/IMGTScientificChart/Numbering/Hu_lGHGnber.html which is herein incorporated in its entirety by reference.

### DETAILED DESCRIPTION

The present disclosure provides novel Fc domain variants (e.g., novel binding polypeptides comprising Fc domain variants) having improved binding to Fc receptors. The present disclosure further provides novel Fc domain variants (e.g., binding polypeptides comprising Fc domain variants) comprising at least one substitution or at least 2 substitutions at amino acid positions 251, 267, 268, 298, 314, 330, 339, 373, and 376, according to EU numbering, as compared to a Fc domain parent polypeptide.

The present disclosure also provides nucleic acids encoding Fc domain variants (e.g., novel binding polypeptides comprising Fc domain variants), recombinant expression vectors and host cells for making Fc domain variants (e.g., novel binding polypeptides comprising Fc domain variants), and pharmaceutical compositions comprising the isolated Fc domain variants (e.g., novel binding polypeptides comprising Fc domain variants). Methods of using the Fc domain variants (e.g., novel binding polypeptides comprising Fc domain variants) of the present disclosure to treat one or more diseases or disorders are also provided.

### Definitions

It is to be understood that the methods described in this disclosure are not limited to particular methods and experimental conditions such that methods and conditions can vary. It is also to be understood that the terminology used herein is for the purpose of describing particular embodiments only and is not intended to be limiting.

The experiments described herein, unless otherwise indicated, use conventional molecular and cellular biological and immunological techniques within the skill of the art. Such techniques are well known to the skilled worker and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2^{nd} edition). Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. Any single term, single element, single phrase, group of terms, group of phrases, or group of elements described herein can each be specifically excluded from the claims.

Generally, nomenclature used in connection with cell culture, molecular biology, immunology, microbiology, genetics, protein biology, and chemistry described herein is well-known and commonly used in the art. The nomenclatures used in connection with, and the laboratory procedures and techniques of, analytical chemistry, synthetic organic chemistry, and medicinal and pharmaceutical chemistry described herein are those well-known and commonly used in the art.

For the disclosure to be more readily understood, select terms are defined below.

### Polypeptide and Isolated Polypeptide

The term “polypeptide” refers to any polymeric chain of amino acids and encompasses native or artificial proteins, polypeptide analogs or variants of a protein sequence, or fragments thereof, unless otherwise contradicted by context. A polypeptide can be monomeric or polymeric. For a polypeptide (e.g., a Fc domain variant polypeptide comprising at least one substitution or at least two substitutions at amino acid positions 251, 267, 268, 298, 314, 330, 339, 373, and 376, according to EU numbering, as compared to a Fc domain parent polypeptide) a fragment of a polypeptide optionally contains at least one contiguous or nonlinear epitope of a polypeptide. A fragment polypeptide can be about 25, 50, 75, 100, 150, 200, 250, 300, 350, 400 or more amino acids in length while retaining the capacity to bind to Fc receptors. The precise boundaries of the at least one epitope fragment can be confirmed using ordinary skill in the art. A polypeptide fragment comprises at least about 5 contiguous amino acids, at least about 10 contiguous amino acids, at least about 15 contiguous amino acids, or at least about 20 contiguous amino acids, at least about 50 contiguous amino acids, at least about 100 contiguous amino acids, at least about 150 contiguous amino acids, at least about 200 contiguous amino acids, at least about 250 contiguous amino acids, at least about 300 contiguous amino acids, at least about 400 contiguous amino acids for example.

In certain aspects, a Fc domain variant polypeptide or a Fc domain parent polypeptide are “isolated polypeptides.” The term “isolated polypeptide” refers to a protein or polypeptide that by virtue of its origin or source of derivation is not associated with naturally associated components that accompany it in its native state, is substantially free of other proteins from the same species. An isolated recombinant polypeptide can be expressed by a cell that does not naturally express the recombinant polypeptide. In some aspects, an isolated polypeptide does not occur in nature. A protein or polypeptide that is chemically synthesized or synthesized in a cellular system can be different from the cell from which it naturally originates and therefore will be “isolated” from its naturally associated components. A protein or polypeptide can also be rendered substantially free of naturally associated components by isolation using protein purification techniques.

Native (Wild-Type) Residue Vs. Variant Polypeptide

As used herein, the term “native residue,” “wild-type residue,” “parental residue” refers to an amino acid residue that occurs naturally at a particular amino acid position of a binding polypeptide (e.g., a wild-type IgG1 or IgG4 Fc domain) and which has not been modified, introduced, or altered by the hand of man. Accordingly, the “parental polypeptide” can refer to a wild-type amino acid sequence encoding a binding polypeptide (e.g., a wild-type IgG1 or IgG4 Fc domain).

The “parental polypeptide” can refer to an amino acid sequence which has been modified but still serves as the reference binding polypeptide when compared to the variant binding polypeptide. For example, a Fc domain parent polypeptide can be a humanized IgG Fc domain. In another example, the parental polypeptide sequence was altered to remove the C′ terminal amino acid residue. In another example, the parental polypeptide sequence had an amino acid substitution, e.g., the parental polypeptide sequence is:

(SEQ ID NO: 50)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG.

As used herein, the term “altered binding protein,” “altered binding polypeptide,” “modified binding protein,” “modified binding polypeptide,” “variant polypeptide,” “mutant polypeptide,” or “engineered polypeptide” refers to binding polypeptides and/or binding proteins (e.g., an antibody or fragment thereof) comprising at least one amino acid substitution, deletion, and/or addition relative to the native (i.e., wild-type) amino acid sequence, and/or a mutation that results in altered glycosylation (e.g., hyperglycosylation, hypoglycosylation and/or aglycosylation) at one or more amino acid positions relative to the native, parental (i.e., wild-type) amino acid sequence.

In one aspect, a Fc domain parent polypeptide comprises a wild-type human IgG1 Fc domain.

In one aspect, the Fc domain parent polypeptide is a wild-type Fc domain comprising a human IgG1 constant domains (CH1-Hinge-CH2-CH3) as set forth in the following amino acid sequence:

(SEQ ID NO: 51)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

In one aspect, the Fc domain parent polypeptide is a wild-type Fc domain comprising a human IgG1 constant domains (CH1-Hinge-CH2-CH3) as set forth in the amino acid sequence above without the C-terminal lysine (K) as set forth in the following amino acid sequence:

(SEQ ID NO: 52)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG.

In one aspect, the Fc domain parent polypeptide is a Fc domain comprising human IgG1 Fc (Hinge-CH2-CH3) as set forth in the following amino acid sequence:

(SEQ ID NO: 53)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK.

In one aspect, the Fc domain parent polypeptide is a Fc domain comprising human IgG1 Fc (Hinge-CH2-CH3) as set forth in the amino acid sequence above but without the C-terminal lysine (K) as set forth in the following amino acid sequence:

(SEQ ID NO: 54)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPG.

In one aspect, the Fc domain parent polypeptide is a Fc domain comprising human IgG1 Fc which comprises an amino acid residue substitution from a cysteine (C) to a serine (S) and does not include a C-terminal lysine (K) resulting in as set forth in the following amino acid sequence:

(SEQ ID NO: 50)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG.

In one aspect, a Fc domain parent polypeptide comprises a wild-type human IgG4 Fc domain.

In one aspect, the Fc domain parent polypeptide is a Fc domain comprising human IgG4P constant domains (CH1-Hinge-CH2-CH3) as set forth in the following amino acid sequence:

(SEQ ID NO: 55)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK.

In one aspect, the Fc domain parent polypeptide is a Fc domain comprising human IgG4P constant domains (CH1-Hinge-CH2-CH3) as set forth in the amino acid sequence above but without the C-terminal lysine (K) as set forth in the following amino acid sequence:

(SEQ ID NO: 56)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLG.

In one aspect, the Fc domain parent polypeptide is a Fc domain comprising human IgG4P Fc (Hinge-CH2-CH3) constant domains as set forth in the following amino acid sequence:

(SEQ ID NO: 57)
ESKYGPPCPP CPAPEFLGGP SVFLFPPKPK DTLMISRTPE
VTCVVVDVSQEDPEVQFNWY VDGVEVHNAK TKPREEQFNS
TYRVVSVLTV LHQDWLNGKEYKCKVSNKGL PSSIEKTISK
AKGQPREPQV YTLPPSQEEMTKNQVSLTCLVKGFYPSDIA
VEWESNGQPE NNYKTTPPVL DSDGSFFLYS
RLTVDKSRWQEGNVFSCSVM HEALHNHYTQ KSLSLSLGK.

In one aspect, the Fc domain parent polypeptide is a Fc domain comprising human IgG4P Fc (Hinge-CH2-CH3) constant but without the C-terminal lysine (K) as set forth in the following amino acid sequence:

(SEQ ID NO: 58)
ESKYGPPCPP CPAPEFLGGP SVFLFPPKPK DTLMISRTPE
VTCVVVDVSQEDPEVQFNWY VDGVEVHNAK TKPREEQFNS
TYRVVSVLTV LHQDWLNGKEYKCKVSNKGL PSSIEKTISK
AKGQPREPQV YTLPPSQEEM TKNQVSLTCLVKGFYPSDIA
VEWESNGQPE NNYKTTPPVL DSDGSFFLYS
RLTVDKSRWQEGNVFSCSVM HEALHNHYTQ KSLSLSLG.

In one aspect, the Fc domain parent polypeptide is encoded by an amino acid sequence comprising at least 80%, 85%, at least 90%, at least 95%, at least 96%, at least 97%, at least 98%, or at least 99% sequence identity to an amino acid sequence set forth in SEQ ID Nos: 50-58.

In one aspect, the Fc domain parent polypeptide is encoded by an amino acid sequence comprising at least 80%, 85%, at least 90%, at least 95%, at least 96%, at least 97%, at least 98%, or at least 99% sequence identity to the amino acid sequence set forth in SEQ ID No: 50.

In one aspect, the Fc domain parent polypeptide is encoded by an amino acid sequence comprising at least 80%, 85%, at least 90%, at least 95%, at least 96%, at least 97%, at least 98%, or at least 99% sequence identity to the amino acid sequence set forth in SEQ ID No: 51.

In one aspect, the Fc domain parent polypeptide is encoded by an amino acid sequence comprising at least 80%, 85%, at least 90%, at least 95%, at least 96%, at least 97%, at least 98%, or at least 99% sequence identity to the amino acid sequence set forth in SEQ ID No: 52.

In one aspect, the Fc domain parent polypeptide is encoded by an amino acid sequence comprising at least 80%, 85%, at least 90%, at least 95%, at least 96%, at least 97%, at least 98%, or at least 99% sequence identity to the amino acid sequence set forth in SEQ ID No: 53.

In one aspect, the Fc domain parent polypeptide is encoded by an amino acid sequence comprising at least 80%, 85%, at least 90%, at least 95%, at least 96%, at least 97%, at least 98%, or at least 99% sequence identity to the amino acid sequence set forth in SEQ ID No: 54.

In one aspect, the Fc domain parent polypeptide is encoded by an amino acid sequence comprising at least 80%, 85%, at least 90%, at least 95%, at least 96%, at least 97%, at least 98%, or at least 99% sequence identity to the amino acid sequence set forth in SEQ ID No: 55.

In one aspect, the Fc domain parent polypeptide is encoded by an amino acid sequence comprising at least 80%, 85%, at least 90%, at least 95%, at least 96%, at least 97%, at least 98%, or at least 99% sequence identity to the amino acid sequence set forth in SEQ ID No: 56.

In one aspect, the Fc domain parent polypeptide is encoded by an amino acid sequence comprising at least 80%, 85%, at least 90%, at least 95%, at least 96%, at least 97%, at least 98%, or at least 99% sequence identity to the amino acid sequence set forth in SEQ ID No: 57.

In one aspect, the Fc domain parent polypeptide is encoded by an amino acid sequence comprising at least 80%, 85%, at least 90%, at least 95%, at least 96%, at least 97%, at least 98%, or at least 99% sequence identity to the amino acid sequence set forth in SEQ ID No: 58.

### Binding Protein or Binding Polypeptide

As used herein, the term “binding protein,” “binding polypeptide,” or “multispecific binding polypeptide or protein” refer to a protein or polypeptide (e.g., an antibody or an antigen binding fragment thereof) that contains at least one binding site which is responsible for selectively binding to a target protein of interest (e.g., a human antigen) as well as a Fc domain (e.g., a wild-type Fc domain or a Fc domain variant as described herein). Exemplary binding sites include an antibody variable domain, a ligand binding site of a receptor, or a receptor binding site of a ligand. In certain aspects, the binding proteins or binding polypeptides comprise multiple (e.g., two, three, four, or more) binding sites. In one aspect, a binding polypeptide can have one or more binding sites for an antigen and one or more binding sites for a Fc receptor. In another aspect, a binding protein or binding polypeptide is not a therapeutic enzyme.

### Ligand and Antigen

The term “ligand” refers to any substance capable of binding, or of being bound, to another substance. The term “antigen” or “target antigen” as used herein refers to a molecule or a portion of a molecule that is capable of being bound by the binding site of a binding polypeptide e.g., any substance to which an antibody can be generated. A target antigen can have one or more epitopes.

Although “antigen” is commonly used in reference to an antibody binding substrate, and “ligand” is often used when referring to receptor binding substrates, these terms are not distinguishing, one from the other, and encompass a wide range of overlapping chemical entities. For the avoidance of doubt, antigen and ligand are used interchangeably throughout herein.

Examples of antigens/ligands can be a peptide, a polypeptide, a protein, an aptamer, a polysaccharide, a sugar molecule, a carbohydrate, a lipid, an oligonucleotide, a polynucleotide, a synthetic molecule, an inorganic molecule, an organic molecule, and any combination thereof.

### Immunoglobulin Domain

The term immunoglobulin domain as used herein can refer to an immunoglobulin A (IgA), an immunoglobulin D (IgD), an immunoglobulin E (IgE), an immunoglobulin G (IgG), or an immunoglobulin M (IgM). The immunoglobulin domain can be an immunoglobulin heavy chain region or fragment thereof. In some instances, the immunoglobulin domain is from an antibody (e.g., a mammalian antibody, a recombinant antibody, a chimeric antibody, an engineered antibody, a human antibody, a humanized antibody) or an antigen binding fragment thereof.

In one aspect, a Fc domain variant polypeptide or a Fc domain parental polypeptide is derived from an immunoglobulin class selected from a group consisting of: IgM, IgG, IgD, IgA, and IgE.

In one aspect, a Fc domain variant polypeptide or a Fc domain parental polypeptide is derived from an IgG Fc domain. In another aspect, a Fc domain variant polypeptide or a Fc domain parental polypeptide is derived from a IgG1 Fc domain or an IgG4 Fc domain.

### Fc Domain Polypeptide

In one aspect, Fc domain polypeptides, e.g., Fc domain variant polypeptides, are provided. As used herein, the term “Fc region” or “Fc domain” refers to the portion of a heavy chain constant region beginning in the hinge region just upstream of the papain cleavage site (i.e., residue 216 in IgG, taking the first residue of heavy chain constant region to be 114) and ending at the C-terminus of the antibody. Accordingly, a complete Fc region comprises at least a hinge domain, a CH2 domain, and a CH3 domain.

The Fc domain of an antibody is involved in non-antigen binding and can mediate effector function by binding to a Fc receptor. There are several different types of Fc receptors, which are classified based on the type of antibody that they recognize. For example, Fc-gamma receptors (FcγR) bind to IgG class antibodies, Fc-alpha receptors (FcαR) bind to IgA class antibodies, and Fc-epsilon receptors (FcεR) bind to IgE class antibodies. The neonatal Fc receptor (FcRn) interacts with the Fc region of an antibody to promote antibody recycling through rescue of normal lysosomal degradation. The FcγRs belong to a family that includes several members, e.g., FcγRI, FcγRIIa, FcγRIIb, FcγRIIIa, and FcγRIIIb.

In one aspect, a Fc domain variant polypeptide is of human origin. In one aspect, a Fc domain variant polypeptide is derived from an immunoglobulin class selected from a group consisting of: IgM, IgG, IgD, IgA, and IgE. In one aspect, a Fc domain variant polypeptide is derived from an IgG Fc domain. In one aspect, a Fc domain polypeptide is derived from a IgG1 Fc domain or an IgG4 Fc domain.

In an aspect, a human IgG1 Fc domain comprises:

(SEQ ID NO: 50)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG.

In an aspect, the fifth amino acid is a serine (S).

In an aspect, a human IgG1 Fc domain comprises:

(SEQ ID NO: 51)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

In an aspect, the C-terminal lysine (K) is deleted.

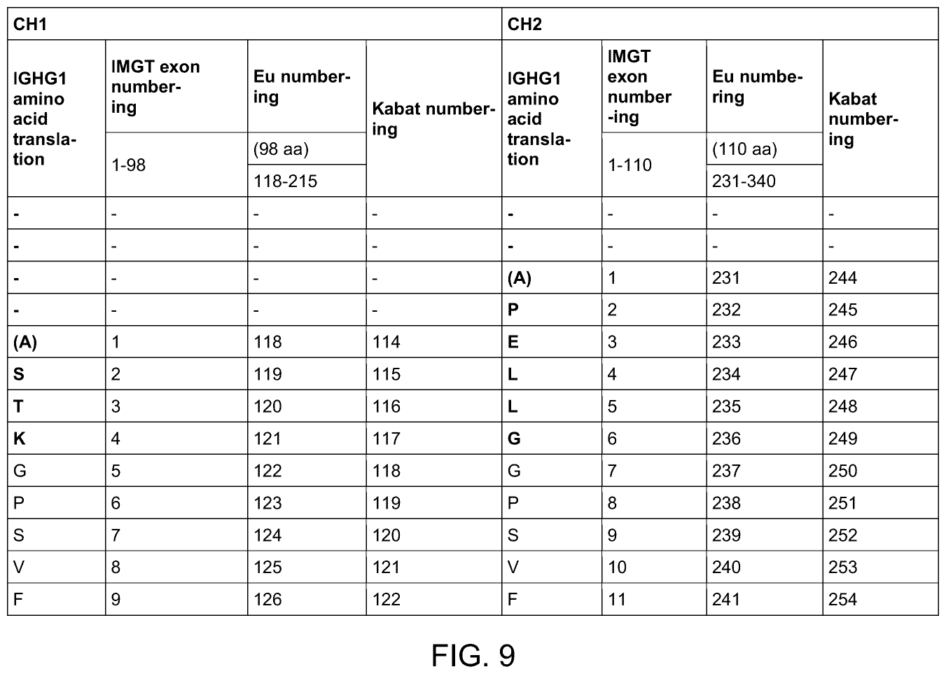

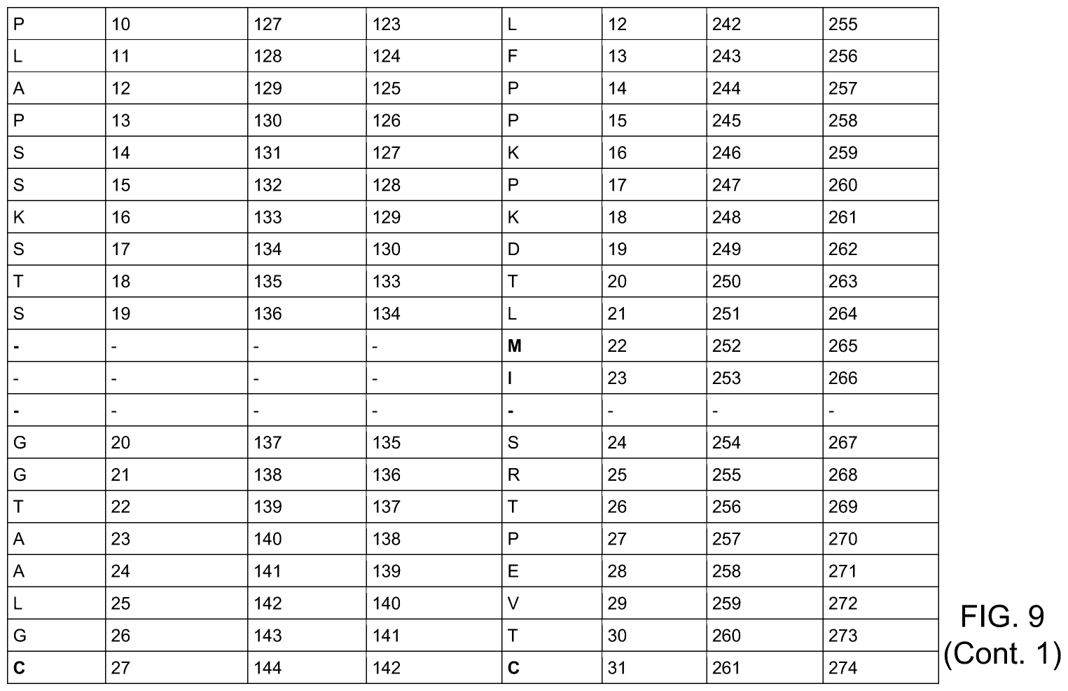

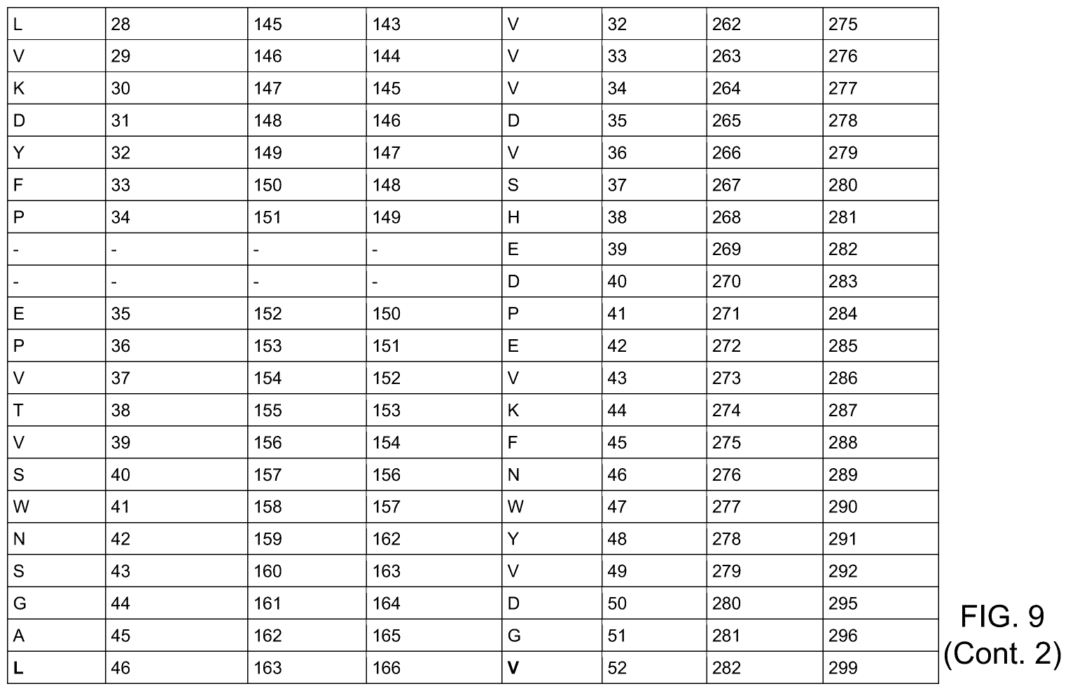

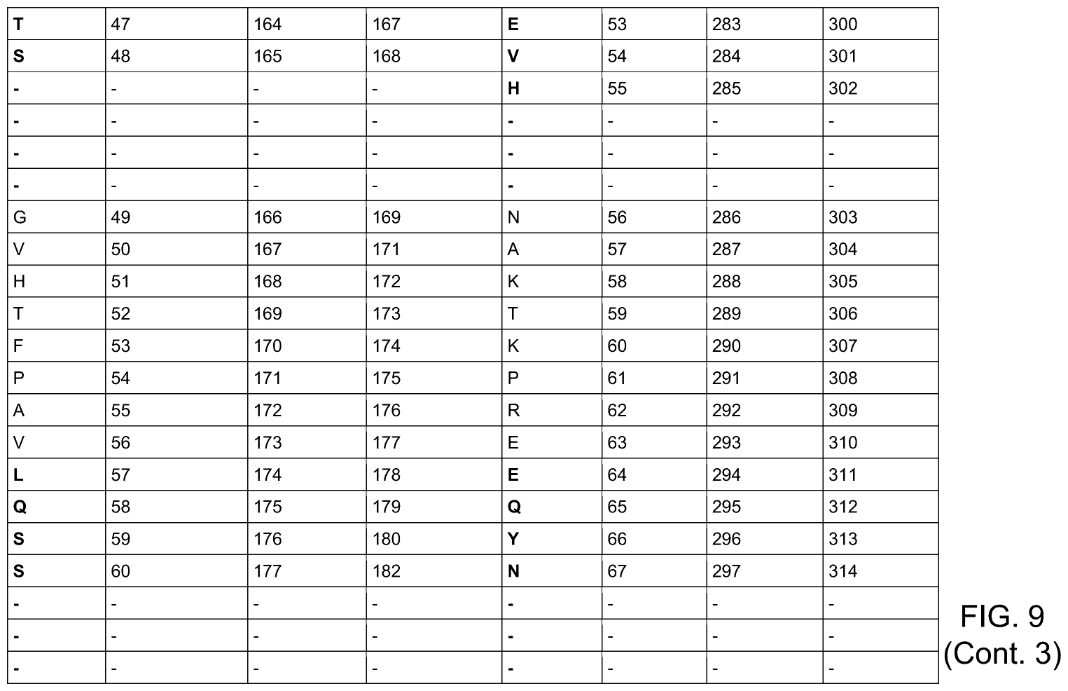

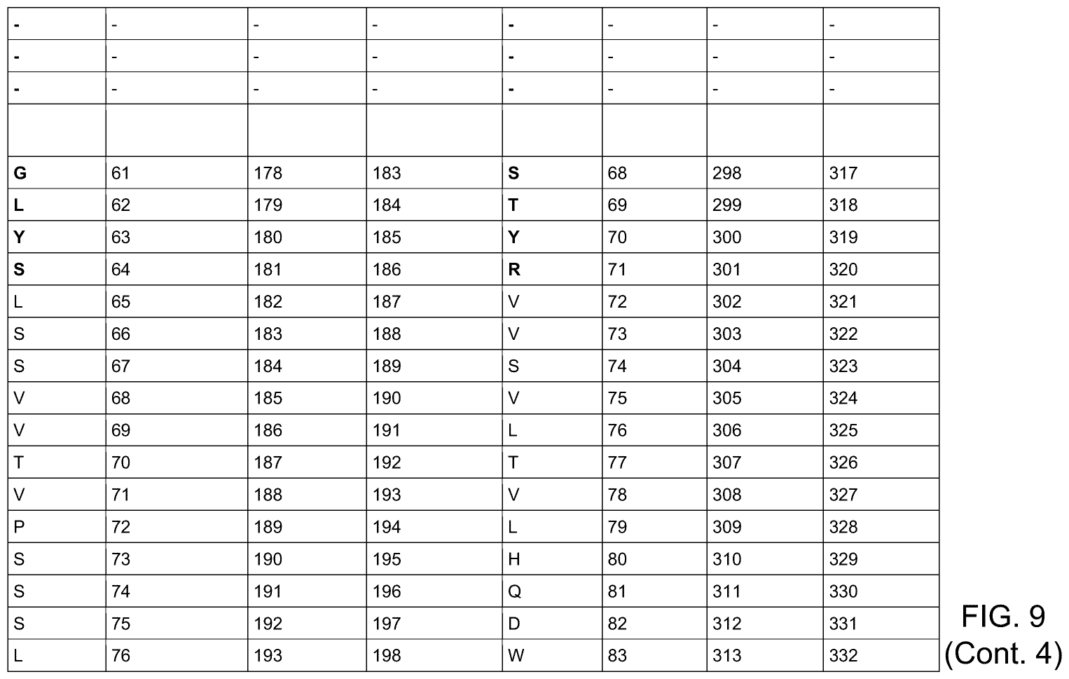

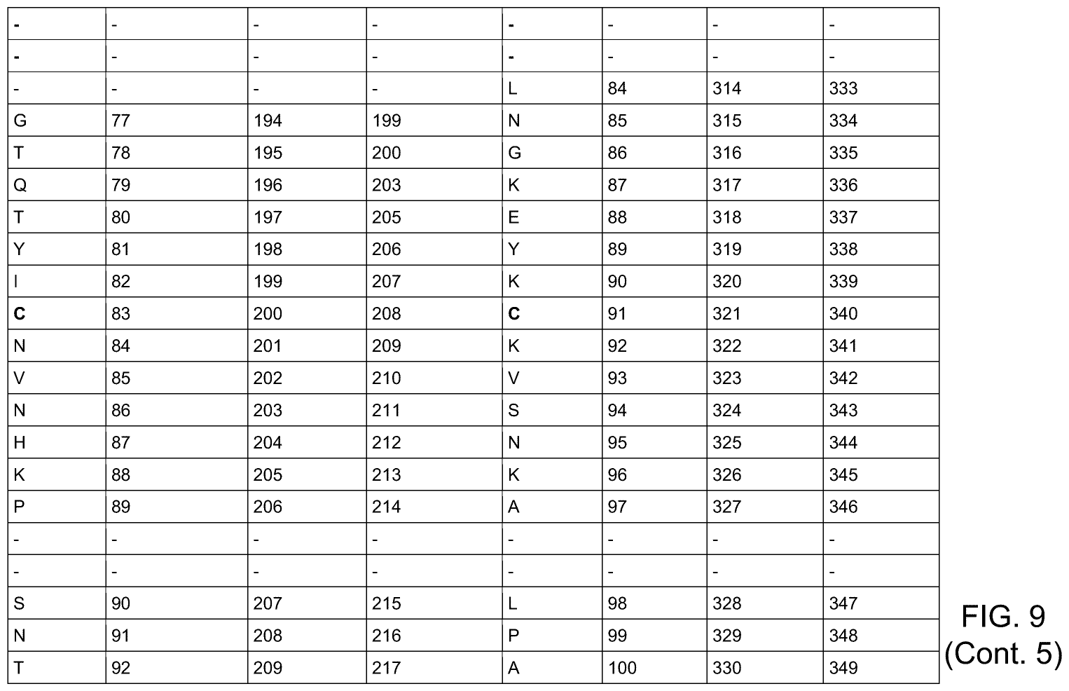

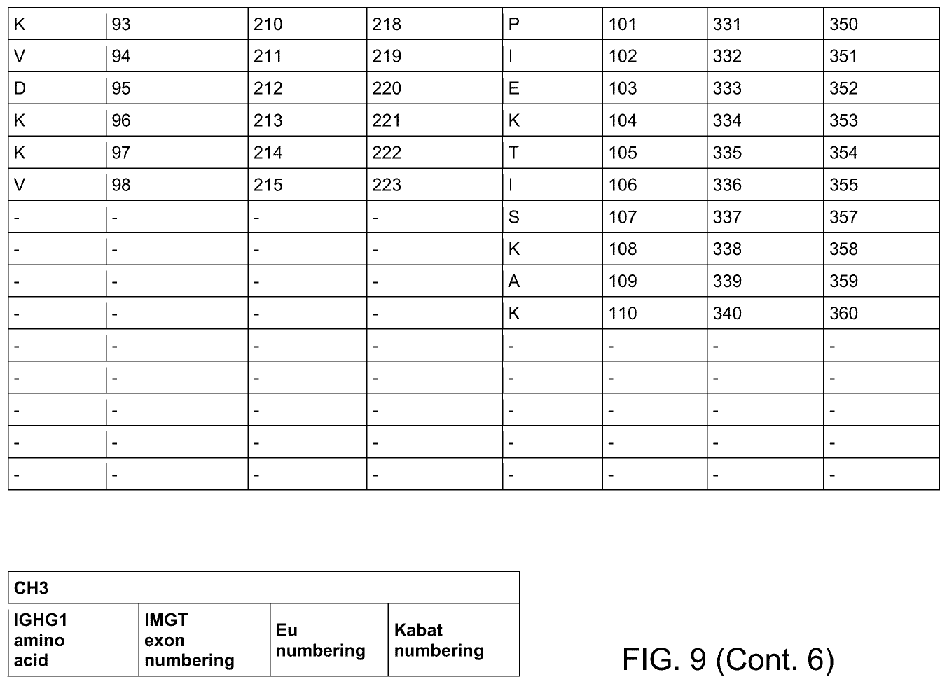

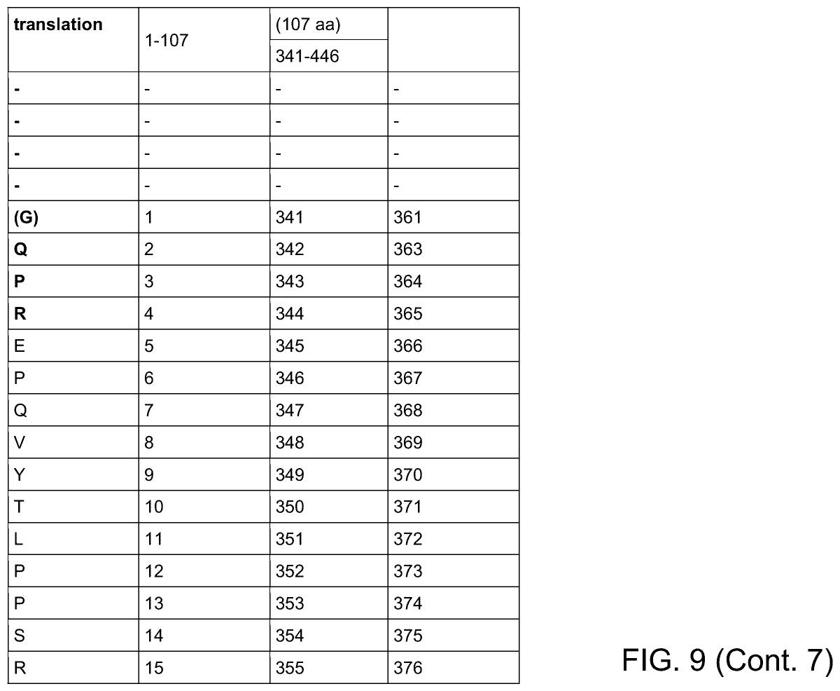

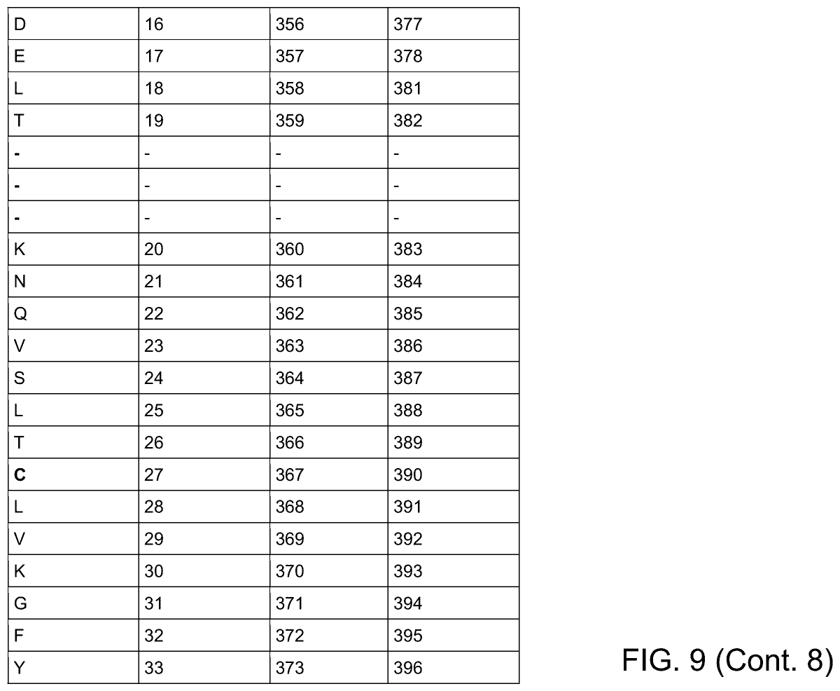

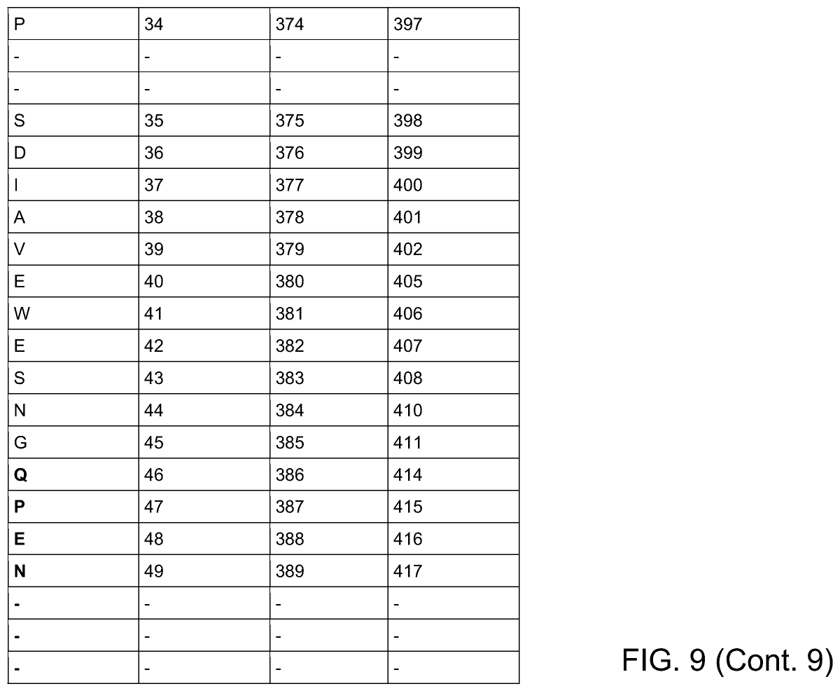

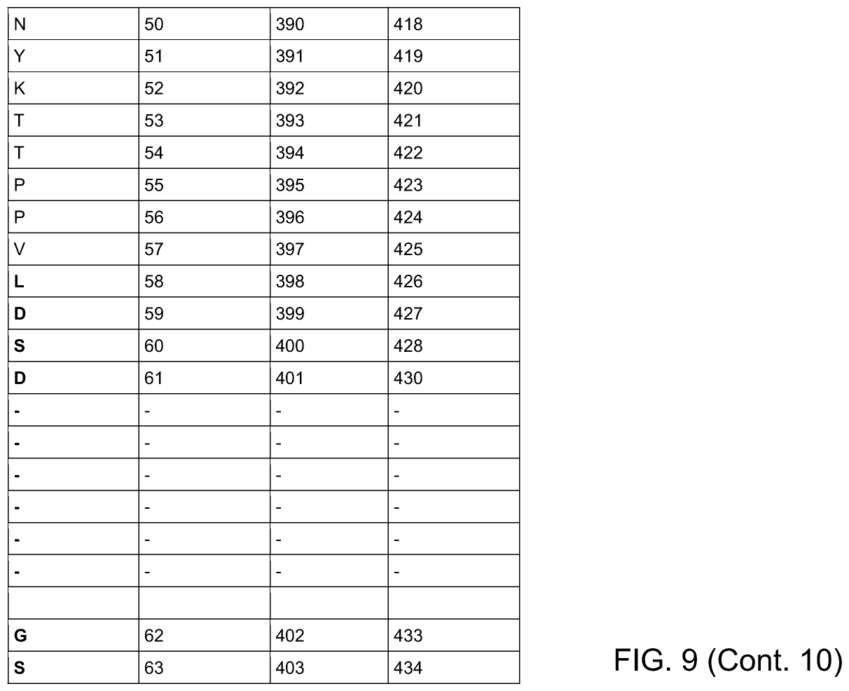

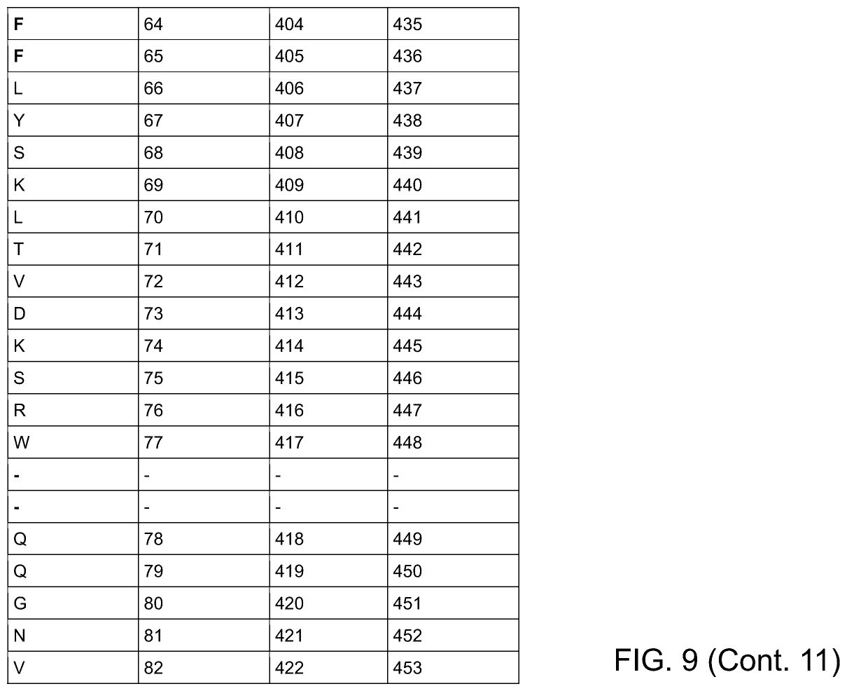

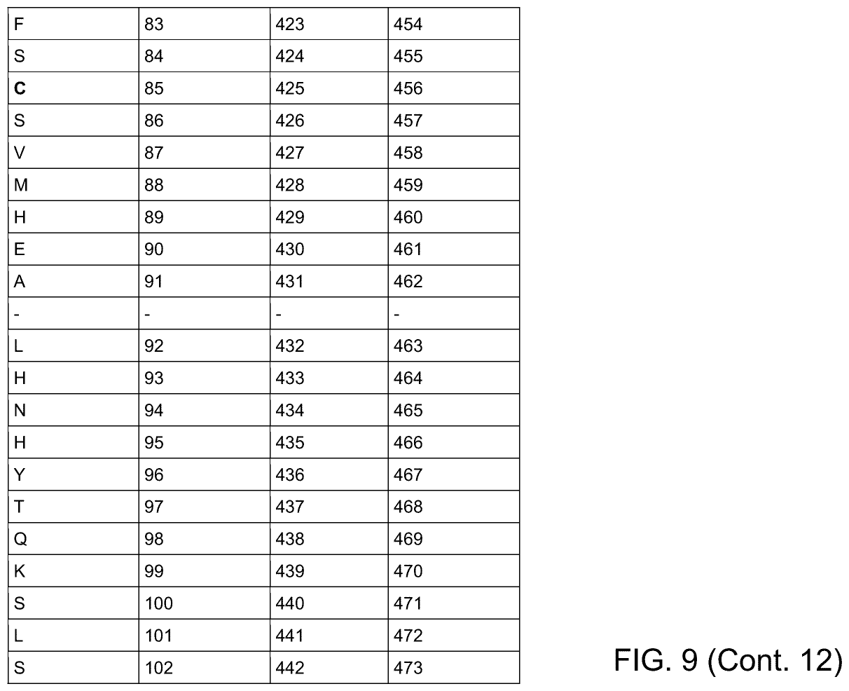

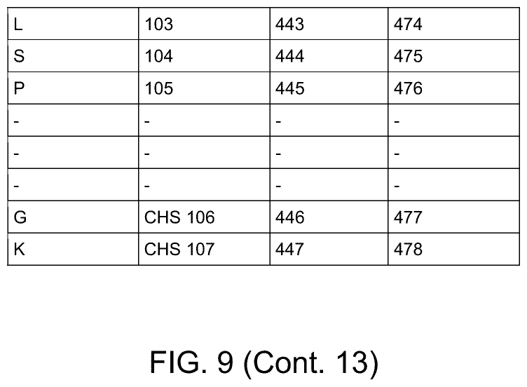

Note, in some aspects, the amino acid residue positions are expressed in EU numbering as shown in Figure. 9. For example, in SEQ ID NO: 50, the M amino acid at position 220 of SEQ ID NO:50 is amino acid 428 according to EU numbering.

### FcγRIIIa

FcγRIIIa V158, or human CD16a-V receptor, or CD16aV, refers to a polypeptide construct comprising a fragment of the CD16 human receptor binding to a Fc region of a natural antibody, mediating antibody-dependent cellular cytotoxicity and bearing a Valine (V) on position 158, which is also reported in the literature as allotype CD16a V158.

FcγRIIIa F158, or human CD16a-F receptor, or CD16aF, refers to a polypeptide construct comprising a fragment of the CD16 human receptor binding to a Fc region of a natural antibody, mediating antibody-dependent cellular cytotoxicity and bearing a phenylalanine (F) on position 158, which is also known as as allotype CD16a F158.

In one aspect, a Fc domain variant polypeptide comprises at least two substitutions at amino acid positions 251, 267, 268, 298, 314, 330, 339, 373, and 376, according to EU numbering, as compared to a Fc domain parent polypeptide. In another aspect, a Fc domain variant polypeptide has increased binding to a Fc receptor relative to the Fc domain parent polypeptide. In another aspect, a Fc receptor is a human Fc receptor. In another aspect, a Fc receptor is a Fcγ receptor (FcγR). In another aspect, a FcγR is a FcγRIIIa or a FcγRIIIb. In another aspect, a FcγRIIIa is hFcγRIIIa with a valine at amino acid position 158, according to EU numbering (FcγRIIIaᵥ₁₅₈). In another aspect, a FcγRIIIa is hFcγRIIIa with a phenylalanine at amino acid position 158, according to EU numbering (FcγRIIIa_{F158}).

### Fc Domain Parent Polypeptide

The term “native Fc,” or “wild-type Fc” or “Fc domain parent polypeptide” as used herein, refers to a molecule corresponding to the sequence of a non-antigen-binding fragment resulting from digestion of an antibody or produced by other means, whether in monomeric or multimeric form, and can contain the hinge region. The original immunoglobulin source of the native Fc is typically of human origin and can be any of the immunoglobulins, such as IgG1 and IgG2. A native Fc can also be of a non-human species origin but has been modified to, e.g., a mouse immunoglobulin that has been humanized.

Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, and IgGA2). One example of a native Fc is a disulfide-bonded dimer resulting from papain digestion of an IgG. The term “native Fc” or “wild-type Fc” as used herein, is generic to the monomeric, dimeric, and multimeric forms.

In one aspect, a Fc domain parent polypeptide is a wild-type Fc domain. In one aspect, a Fc domain parent polypeptide is a wild-type Fc IgG domain. In one aspect, a Fc domain parent polypeptide is derived from a commercially available antibody. In another aspect, a Fc domain parent polypeptide is derived from a modified Fc domain.

In one aspect, a Fc domain parent polypeptide comprises at least one modified glycan. In one aspect, a Fc domain parent polypeptide comprises at least one N-glycan. In another aspect, a Fc domain parent polypeptide comprises a bis mannose 6 phosphate (bisM6P), a disaccharide mannose 6 phosphate, or a mannose 6 phosphate monosaccharide. In another aspect, a Fc domain parent polypeptide comprises an oligomannose-type N-glycan comprising an oligosaccharide selected from the group consisting of: a Man₉(GlcNAc)₂, a Man₈(GlcNAc)₂, a Man₇(GlcNAc)₂, a Man₆(GlcNAc)₂, or a Man₅(GlcNAc)₂. In another aspect, a Fc domain parent polypeptide is about 20%, 30%, 40%, 50%, 60%, 70%, 90% or more afucosylated.

In one aspect, a Fc domain variant polypeptide comprising at least one substitution or at least two substitutions at amino acid positions 251, 267, 268, 298, 314, 330, 339, 373, and 376, according to EU numbering, as compared to a Fc domain parent polypeptide.

### Fc Domain Variant Polypeptide and Fc Domain

The term “Fc domain variant,” “Fc variant,” “modified Fc,” or “Fc domain variant polypeptide” as used herein, refers to a molecule or sequence that is modified from a native/wild-type Fc, but still comprises a binding site for a Fc receptor. Thus, the term “Fc variant” can comprise a molecule or sequence that is humanized from a non-human native Fc. Furthermore, a native Fc comprises regions that can be removed because they provide structural features or biological activities that are not required for the antibody-like binding polypeptides described herein. Thus, the term “Fc domain variant polypeptide” comprises a molecule or sequence that lacks one or more native Fc sites or residues, or in which one or more Fc sites or residues has been modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to a Fc receptor (e.g., FcγRIIIa) other than a salvage receptor, or (7) antibody-dependent cellular cytotoxicity (ADCC).

As used herein, an “effector-competent Fc domain variant” or “effector-competent polypeptide” refers to a Fc domain variant polypeptide that has one or more Fc effector functions as described further herein.

In one aspect, a Fc domain variant polypeptide featured herein has one or more of increased serum half-life, enhanced FcRn binding affinity, enhanced FcRn binding affinity at acidic pH, enhanced FcγRIIIa binding affinity, and/or similar thermal stability, as compared to a Fc domain parent polypeptide.

The term “Fc domain” as used herein encompasses a Fc domain parent polypeptide (e.g., a native/wild-type Fc domain) and a Fc domain variant polypeptide and sequences as defined herein. As with Fc domain variant polypeptides and Fc domain parent polypeptides (e.g., native Fc domain molecules), the term “Fc domain” includes molecules in monomeric or multimeric form, whether digested from whole antibody or produced by other means.

In one aspect, a Fc domain variant polypeptide as described herein is thermally stabilized. Exemplary Fc domain variant polypeptides that can enhance thermal stability are described in U.S. Prov. Patent Application Ser. No. 63/193,655 which is incorporated by reference in its entirety.

In one aspect, a Fc domain as described herein comprises at least one N-glycan. In one aspect, a Fc domain as described herein is glycosylated (e.g., via N-linked glycosylation). In one aspect, an Fc domain comprises N-linked glycosylation, e.g., at an N-linked glycosylation motif that contains the amino acid sequence NXT or NXS (X being any amino acid residue except proline). In certain exemplary embodiments, a Fc domain is glycosylated at amino acid position 297, according to EU numbering.

In one aspect, a Fc domain as described herein is effector-competent.

In one aspect, a Fc domain as described herein is any combination of thermally stabilized, glycosylated, and effector-competent.

### Effector-Enhancing Fc Domain Variants

In one aspect, the present disclosure provides a Fc domain variant polypeptide comprising effector-enhancing amino acid substitutions.

In one aspect, a Fc domain variant polypeptide has altered binding affinity to a Fc receptor. There are several different types of Fc receptors, which are classified based on the type of antibody that they recognize. For example, Fc-gamma receptors (FcγR) bind to IgG class antibodies, Fc-alpha receptors (FcaR) bind to IgA class antibodies, and Fc-epsilon receptors (FcεR) bind to IgE class antibodies. The FcγRs belong to a family that includes several members, e.g., FcγRI, FcγRIIa, FcγRIIb, FcγRIIIa, and FcγRIIIb.

In one aspect, a Fc domain variant polypeptide has altered FcγRIIIa binding affinity, compared to a Fc domain parent polypeptide. In another aspect, a Fc domain variant has reduced FcγRIIIa binding affinity, compared to a wild-type Fc domain polypeptide. In another aspect, a Fc domain variant has enhanced FcγRIIIa binding affinity, compared to a wild-type IgG Fc domain polypeptide. In another aspect, a Fc domain variant polypeptide modified Fc domain has approximately the same FcγRIIIa binding affinity, compared to a Fc domain parent polypeptide.

In another aspect, a Fc domain variant polypeptide with altered FcγRIIIa binding comprising one or more amino acid substitutions (e.g., 1, 2, 3, 4, 5 or more) is disclosed herein. In another aspect, a Fc domain variant polypeptide with enhanced FcγRIIIa binding affinity has one or more amino acid substitutions as disclosed herein. In one aspect, a Fc domain variant polypeptide with enhanced FcγRIIIa binding affinity comprises two or more amino acid substitutions as disclosed herein. In one aspect, a Fc domain variant polypeptide with enhanced FcγRIIIa binding affinity comprises three or more amino acid substitutions as disclosed herein. In one aspect, a Fc domain variant polypeptide with enhanced FcγRIIIa binding affinity comprises four or more amino acid substitutions as disclosed herein.

In one aspect, a Fc domain variant polypeptide with altered FcRn binding comprises a Fc domain having one or more amino acid substitutions (e.g., 1, 2, 3, 4, 5 or more) as disclosed herein. In another aspect, a Fc domain variant polypeptide with enhanced FcRn binding affinity comprises a Fc domain having one or more amino acid substitutions as disclosed herein. In another aspect, a Fc domain variant polypeptide with enhanced FcRn binding affinity comprises a Fc domain having two or more amino acid substitutions as disclosed herein. In another aspect, a Fc domain variant polypeptide with enhanced FcRn binding affinity comprises a Fc domain having three or more amino acid substitutions as disclosed herein. In one aspect, a Fc domain variant polypeptide with enhanced FcRn binding affinity comprises four or more amino acid substitutions as disclosed herein.

In one aspect, a Fc domain variant polypeptide may exhibit a species-specific FcRn binding affinity. In one aspect, a Fc domain variant polypeptide may exhibit human FcRn binding affinity. In one aspect, a Fc domain variant polypeptide may exhibit cyno FcRn binding affinity. In another aspect, a Fc domain variant polypeptide may exhibit cross-species FcRn binding affinity. Such a Fc domain variant polypeptide is said to be cross-reactive across one or more different species. In another aspect, a Fc domain variant may exhibit both human and cyno FcRn binding affinity.

In certain embodiments, a Fc domain variant polypeptide comprises an antibody constant region (e.g., an IgG constant region e.g., a human IgG constant region, e.g., a human IgG1 constant region) which mediates one or more effector functions. For example, binding of the C1-complex to an antibody constant region may activate the complement system. Activation of the complement system is important in the opsonization and lysis of cell pathogens. The activation of the complement system also stimulates the inflammatory response and may also be involved in autoimmune hypersensitivity.

Furthermore, antibodies and antibodies comprising a Fc domain variant polypeptide can bind to receptors on various cells via the Fc domain (Fc receptor binding sites on the antibody Fc region bind to Fc receptors (FcRs) on a cell). There are a number of Fc receptors which are specific for different classes of antibody, including IgG (gamma receptors), IgE (epsilon receptors), IgA (alpha receptors) and IgM (mu receptors). Binding of antibody and/or Fc domain variant polypeptides to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. In one aspect, a Fc domain variant polypeptide, e.g., a binding polypeptide (e.g., an antibody or immunoadhesin) binds to a Fc-gamma (Fcγ) receptor. In another aspect, a Fc domain variant polypeptide comprised a constant region which is devoid of one or more effector functions (e.g., ADCC activity) and/or is unable to bind Fcγ receptor.

### Amino Acid Substitutions

In one aspect, a Fc domain variant polypeptide comprises at least one substitution (e.g., 1, 2, 3, 4, 5 or more) at amino acid positions 251, 267, 268, 298, 314, 330, 339, 373, and 376, according to EU numbering, as compared to a Fc domain parent polypeptide. In another aspect, a Fc domain variant polypeptide comprises at least one or two substitutions at amino acid position 251 or 376, according to EU numbering, as compared to a Fc domain parent polypeptide. In another aspect, a Fc domain variant polypeptide comprises at least two substitutions at amino acid positions 251, 267, 268, 298, 314, 330, 339, 373, and 376, according to EU numbering, as compared to a Fc domain parent polypeptide.

In one aspect, a Fc domain variant polypeptide comprises at least two amino acid substitutions selected from: (i) an alanine (A), a histidine (H), an isoleucine (I), a phenylalanine (F), a glutamine (Q), or a tryptophan (W) at amino acid position 251, (ii) an alanine (A) or an aspartic acid (D) at amino acid position 267, (iii) an aspartic acid (D) or a glutamic acid (E) at amino acid position 268, (iv) an alanine (A) at amino acid position 298, (v) a glycine (G), a lysine (K), an asparagine (N), a methionine (M), a serine (S), a threonine (T), a valine (V), a glutamic acid (E), or a tryptophan (W) at amino acid position 314, (vi) a phenylalanine (F), a methionine (M), or a tyrosine (Y) at amino acid position 330, (vii) a threonine (T) at amino acid position 339, (viii) a tryptophan (W) at amino acid position 373, or (ix) a valine (V) at amino acid position 376, according to EU numbering.

In one aspect, a Fc domain variant polypeptide comprises an aspartic acid (D) at amino acid position 268, a glutamine (Q) at amino acid position 251, a tryptophan (W) at amino acid position 373, and an alanine (A) at amino acid position 298, according to EU numbering.

In one aspect, a Fc domain variant polypeptide comprises an aspartic acid (D) at amino acid position 268, a glutamine (Q) at amino acid position 251, and an alanine (A) at amino acid position 298, according to EU numbering.

In one aspect, a Fc domain variant polypeptide comprises an aspartic acid (D) at amino acid position 268, an isoleucine (I) at amino acid position 251, a phenylalanine (F) at amino acid position 330, and an alanine (A) at amino acid position 298, according to EU numbering.

In one aspect, a Fc domain variant polypeptide comprises an aspartic acid (D) at amino acid position 268, a threonine (T) at amino acid position 339, and an alanine (A) at amino acid position 298, according to EU numbering.

In one aspect, a Fc domain variant polypeptide comprises a glutamic acid (E) at amino acid position 268, a valine (V) at amino acid position 376, an alanine (A) at amino acid position 251, and a methionine (M) at amino acid position 330, according to EU numbering.

In one aspect, a Fc domain variant polypeptide comprises an aspartic acid (D) at amino acid position 268, an aspartic acid (D) at amino acid position 267, an isoleucine (I) at amino acid position 251, and a threonine (T) at amino acid position 339, according to EU numbering.

In one aspect, a Fc domain variant polypeptide comprises an aspartic acid (D) at amino acid position 268, a tyrosine (Y) at amino acid position 330, a threonine (T) at amino acid position 339, a tryptophan (W) at amino acid position 373, according to EU numbering.

In one aspect, a Fc domain variant polypeptide comprises an aspartic acid (D) at amino acid position 268, a glutamine (Q) at amino acid position 251, a tryptophan (W) at amino acid position 373, according to EU numbering.

In one aspect, a Fc domain variant polypeptide comprises an aspartic acid (D) at amino acid position 268 and a threonine (T) at amino acid position 314, according to EU numbering.

In one aspect, a Fc domain variant polypeptide comprises an aspartic acid (D) at amino acid position 268, a phenylalanine (F) at amino acid position 251, and a valine (V) at amino acid position 314, according to EU numbering.

In one aspect, a Fc domain variant comprises an aspartic acid (D) at amino acid position 268, a tyrosine (Y) at amino acid position 330, a valine (V) at amino acid position 376, according to EU numbering.

In one aspect, a Fc domain variant polypeptide comprises an alanine (A) at amino acid position 298, a tyrosine (Y) at amino acid position 330, a threonine (T) at amino acid position 339, and a tryptophan (W) at amino acid position 373, according to EU numbering.

In one aspect, a Fc domain variant polypeptide comprises an aspartic acid (D) at amino acid position 268 and an alanine (A) at amino acid position 251, according to EU numbering.

In one aspect, a Fc domain variant polypeptide comprises an aspartic acid (D) at amino acid position 267, a tyrosine (Y) at amino acid position 330, a threonine (T) at amino acid position 339, according to EU numbering.

In one aspect, a Fc domain variant polypeptide comprises an aspartic acid (D) at amino acid position 267, a tyrosine (Y) at amino acid position 330, a tryptophan (W) at amino acid position 373, according to EU numbering.

In one aspect, a Fc domain variant polypeptide comprises a glutamic acid (E) at amino acid position 268, a histidine (H) at amino acid position 251, a phenylalanine (F) at amino acid position 330, according to EU numbering.

In one aspect, a Fc domain variant polypeptide comprises an aspartic acid (D) at amino acid position 268, a tryptophan (W) at amino acid position 373, an alanine (A) at amino acid position 298, according to EU numbering.

In one aspect, a Fc domain variant polypeptide comprises an aspartic acid (D) at amino acid position 268 and an alanine (A) at amino acid position 298, according to EU numbering.

In one aspect, a Fc domain variant polypeptide comprises an aspartic acid (D) at amino acid position 268, a phenylalanine (F) at amino acid position 330, and an alanine (A) at amino acid position 298, according to EU numbering.

In one aspect, a Fc domain variant polypeptide comprises a glutamic acid (E) at amino acid position 268, a valine (V) at amino acid position 314, a methionine (M) at amino acid position 330, according to EU numbering.

In one aspect, a Fc domain variant polypeptide comprises an aspartic acid (D) at amino acid position 268, an aspartic acid (D) at amino acid position 267, a threonine (T) at amino acid position 339, according to EU numbering.

In one aspect, a Fc domain variant polypeptide comprises an aspartic acid (D) at amino acid position 268 and a tryptophan (W) at amino acid position 373, according to EU numbering.

In one aspect, a Fc domain variant polypeptide comprises an aspartic acid (D) at amino acid position 268 and a valine (V) at amino acid position 314, according to EU numbering.

In one aspect, a Fc domain variant polypeptide comprises a glutamic acid (E) at amino acid position 268 and a phenylalanine (F) at amino acid position 330, according to EU numbering.

In one aspect, a Fc domain variant polypeptide comprises an aspartic acid (D) at amino acid position 268, a glutamine (Q) at amino acid position 251, and a glutamic acid (E) at amino acid position 314, according to EU numbering.

In one aspect, a Fc domain variant polypeptide comprises an aspartic acid (D) at amino acid position 268, a glutamine (Q) at amino acid position 251, a methionine (M) at amino acid position 314, and an alanine (A) at amino acid position 298, according to EU numbering.

In one aspect, a Fc domain variant polypeptide comprises an aspartic acid (D) at amino acid position 268, a glutamine (Q) at amino acid position 251, a tryptophan (W) at amino acid position 373, and an alanine (A) at amino acid position 298, according to EU numbering.

In one aspect, a Fc domain variant polypeptide comprises an aspartic acid (D) at amino acid position 268, a tryptophan (W) at amino acid position 251, and a serine (S) at amino acid position 314, according to EU numbering.

In one aspect, a Fc domain variant polypeptide comprises an aspartic acid (D) at amino acid position 268, a glutamine (Q) at amino acid position 251, and an alanine (A) at amino acid position 298, according to EU numbering.

In one aspect, a Fc domain variant polypeptide comprises an aspartic acid (D) at amino acid position 268, an isoleucine (I) at amino acid position 251, a phenylalanine (F) at amino acid position 330, and an alanine (A) at amino acid position 298, according to EU numbering.

In one aspect, a Fc domain variant comprises an aspartic acid (D) at amino acid position 268, a glutamine (Q) at amino acid position 251, and a methionine (M) at amino acid position 314, and an alanine (A) at amino acid position 298, according to EU numbering.

In one aspect, a Fc domain variant polypeptide comprises an aspartic acid (D) at amino acid position 268, a tryptophan (W) at amino acid position 251, and an asparagine (N) at amino acid position 314 according to EU numbering.

In one aspect, a Fc domain variant polypeptide comprises an aspartic acid (D) at amino acid position 268, a phenylalanine (F) at amino acid position 251, a methionine (M) at amino acid position 314, and an alanine (A) at amino acid position 298, according to EU numbering.

In one aspect, a Fc domain variant polypeptide comprises an aspartic acid (D) at amino acid position 268, a tryptophan (W) at amino acid position 314, a tryptophan (W) at amino acid position 373, and a methionine (M) at amino acid position 330, according to EU numbering.

In one aspect, a Fc domain variant polypeptide comprises a glutamic acid (E) at amino acid position 268, an isoleucine (I) at amino acid position 251, a threonine (T) at amino acid position 314, and a valine (V) at amino acid position 376, according to EU numbering.

In one aspect, a Fc domain variant polypeptide, wherein the Fc domain variant comprises an aspartic acid (D) at amino acid position 268, a glutamine (Q) at amino acid position 251, and a glycine (G) at amino acid position 314, according to EU numbering.

In one aspect, a Fc domain variant polypeptide comprises an aspartic acid (D) at amino acid position 268, a tryptophan (W) at amino acid position 251, and a glycine (G) at amino acid position 314, and an alanine (A) at amino acid position 298, according to EU numbering.

In one aspect, a Fc domain variant polypeptide comprises an aspartic acid (D) at amino acid position 268, a tyrosine (Y) at amino acid position 330, and a lysine (K) at amino acid position 314, according to EU numbering.

In one aspect, a Fc domain variant polypeptide comprises an aspartic acid (D) at amino acid position 268, a tryptophan (W) at amino acid position 251, a threonine (T) at amino acid position 314, and an alanine (A) at amino acid position 298, according to EU numbering.

In one aspect, a Fc domain variant polypeptide comprises a glutamic acid (E) at amino acid position 268, a valine (V) at amino acid position 314, an alanine (A) at amino acid position 251, and a methionine (M) at amino acid position 330, according to EU numbering.

In one aspect, a Fc domain variant polypeptide comprises an alanine (A) at amino acid position 298, a tyrosine (Y) at amino acid position 330, and a threonine (T) at amino acid position 339, according to EU numbering.

In one aspect, a Fc domain variant polypeptide comprises an aspartic acid (D) at amino acid position 268, a phenylalanine (F) at amino acid position 251, and a lysine (K) at amino acid position 314 according to EU numbering.

In one aspect, a Fc domain variant polypeptide comprises an aspartic acid (D) at amino acid position 268, a phenylalanine (F) at amino acid position 251, and a glycine (G) at amino acid position 314, according to EU numbering.

In one aspect, a Fc domain variant polypeptide comprises an aspartic acid (D) at amino acid position 268, a tyrosine (Y) at amino acid position 330, a tryptophan (W) at amino acid position 251, and a valine (V) at amino acid position 376, according to EU numbering.

In one aspect, a Fc domain variant polypeptide comprises a tyrosine (Y) at amino acid position 330, an alanine (A) at amino acid position 267, a valine (V) at amino acid position 376, and an alanine (A) at amino acid position 298, according to EU numbering.

In one aspect, a Fc domain variant polypeptide comprises an aspartic acid (D) at amino acid position 268, a phenylalanine (F) at amino acid position 251, and a threonine (T) at amino acid position 314, according to EU numbering.

In one aspect, a Fc domain variant polypeptide comprises an aspartic acid (D) at amino acid position 268, a tryptophan (W) at amino acid position 251, a methionine (M) at amino acid position 314, and an alanine (A) at amino acid position 298, according to EU numbering.

In one aspect, a Fc domain variant polypeptide comprises an aspartic acid (D) at amino acid position 268, a tyrosine (Y) at amino acid position 330, and a tryptophan (W) at amino acid position 373, according to EU numbering.

In one aspect, a Fc domain variant polypeptide comprises an aspartic acid (D) at amino acid position 268 and a glutamic acid (E) at amino acid position 314, according to EU numbering.

In one aspect, a Fc domain variant polypeptide comprises an aspartic acid (D) at amino acid position 268, a methionine (M) at amino acid position 314, and an alanine (A) at amino acid position 298, according to EU numbering.

In one aspect, a Fc domain variant polypeptide comprises an aspartic acid (D) at amino acid position 268, a tryptophan (W) at amino acid position 373, and an alanine (A) at amino acid position 298, according to EU numbering.

In one aspect, a Fc domain variant polypeptide comprises an aspartic acid (D) at amino acid position 268 and a serine (S) at amino acid position 314, according to EU numbering.

In one aspect, a Fc domain variant polypeptide comprises an aspartic acid (D) at amino acid position 268 and an asparagine (N) at amino acid position 314, according to EU numbering.

In one aspect, a Fc domain variant polypeptide comprises a glutamic acid (E) at amino acid position 268, a threonine (T) at amino acid position 314, and a valine (V) at amino acid position 376, according to EU numbering.

In one aspect, a Fc domain variant polypeptide comprises an aspartic acid (D) at amino acid position 268 and a glycine (G) at amino acid position 314, according to EU numbering.

In one aspect, a Fc domain variant polypeptide comprises an aspartic acid (D) at amino acid position 268, a threonine (T) at amino acid position 314, and an alanine (A) at amino acid position 298, according to EU numbering.

In one aspect, a Fc domain variant polypeptide comprises a glutamic acid (E) at amino acid position 268, a valine (V) at amino acid position 314, and a methionine (M) at amino acid position 330, according to EU numbering.

In one aspect, a Fc domain variant polypeptide comprises an aspartic acid (D) at amino acid position 268 and a lysine (K) at amino acid position 314, according to EU numbering.

In one aspect, a Fc domain variant polypeptide comprises an aspartic acid (D) at amino acid position 268, a tyrosine (Y) at amino acid position 330, and a valine (V) at amino acid position 376, according to EU numbering.

In one aspect, an effector-enhancing Fc domain variant polypeptide has one or more amino acid substitutions selected from the group consisting of: an aspartic acid (D) at amino acid position 221; a cysteine (C) at amino acid position 222; a tyrosine (Y) at amino acid position 234; an alanine (A) at amino acid position 236; a tryptophan (W) at amino acid position 236; an aspartic acid (D) at amino acid position 239; a leucine (L) at amino acid position 243; a glutamic acid (E) at amino acid position 267; a phenylalanine (F) at amino acid position 268; a proline (P) at amino acid position 292; an alanine (A) at amino acid position 298; a leucine (L) at amino acid position 300; an isoleucine (I) at amino acid position 305; a threonine (T) at amino acid position 324; a tryptophan (W) at amino acid position 326; an alanine (A) at amino acid position 326; a leucine (L) at amino acid position 330; a glutamic acid (E) at amino acid position 332; an alanine (A) at amino acid position 333; a serine (S) at amino acid position 333; an alanine (A) at amino acid position 334; an alanine (A) at amino acid position 336; an arginine (R) at amino acid position 345; and a leucine (L) at amino acid position 396, according to EU numbering. See Saunders, 2009, Front. Immunol. doi: 10.3389/fimmu.2019.01296, for a review.

In one aspect, a Fc domain variant polypeptide can comprise an amino acid substitution at positions selected from amino acid positions 236, 239, 330, and 332, according to EU numbering. In one aspect, the substitutions may comprise an alanine (A) at amino acid position 236, an aspartic acid (D) at amino acid 239, a leucine (L) at amino acid position 330, and a glutamic acid (E) at amino acid position 332, according to EU numbering. In one aspect, a Fc domain variant polypeptide can comprise a double amino acid substitution at any two amino acid positions selected from an alanine (A) at amino acid position 236, aspartic acid (D) at amino acid 239, a leucine (L) at amino acid position 330, and a glutamic acid (E) at amino acid position 332. In one aspect, a Fc domain variant can comprise a triple amino acid substitution at any three amino acid positions selected from an alanine (A) at amino acid position 236, an aspartic acid (D) at amino acid 239, a leucine (L) at amino acid position 330, and a glutamic acid (E) at amino acid position 332. In one aspect, a Fc domain variant polypeptide can comprise a quadruple amino acid substitution at any four amino acid positions selected from an alanine (A) at amino acid position 236, an aspartic acid (D) at amino acid 239, a leucine (L) at amino acid position 330, and a glutamic acid (E) at amino acid position 332. In one aspect, a Fc domain variant polypeptide can comprise the combination of amino acid substitutions comprising an aspartic acid (D) at amino acid 239 and a glutamic acid (E) at amino acid position 332. In one aspect, a Fc domain variant polypeptide can comprise the combination of amino acid substitutions comprising an alanine (A) at amino acid position 236, an aspartic acid (D) at amino acid position and a glutamic acid at position 332.

In one aspect, a Fc domain variant polypeptide can further comprise an amino acid substitution at amino acid positions 256 and/or 307, according to EU numbering. In one aspect, a Fc domain variant polypeptide can comprise the combination of amino acid substitutions comprising an aspartic acid (D) at amino acid positions 256 and a glutamine (Q) at amino acid position 307. See Mackness et al., 2019 MAbs 11:1276-88 as well as WO2019147973A1, which are incorporated in its entirety by reference herein.

In one aspect, a Fc domain variant polypeptide comprises an aspartic acid (D) at amino acid position 268, a glutamine (Q) at amino acid position 251, a methionine (M) at amino acid position 314, and an alanine (A) at amino acid position 298, according to EU numbering.

In one aspect, a Fc domain variant polypeptide comprises an aspartic acid (D) at amino acid position 268, an aspartic acid (D) at amino acid position 267, and a threonine (T) at amino acid position 339, according to EU numbering.

In one aspect, a Fc domain variant polypeptide comprises an aspartic acid (D) at amino acid position 268 and a threonine (T) at position 314, according to EU numbering.

In one aspect, a Fc domain variant polypeptide comprises an aspartic acid (D) at amino acid position 268 and a glutamic acid (E) at position 314, according to EU numbering.

In one aspect, a Fc domain variant polypeptide comprises an aspartic acid (D) at amino acid position 268 and a glycine (G) at position 314, according to EU numbering.

In one aspect, a Fc domain variant polypeptide comprises an aspartic acid (D) at amino acid position 268 and a serine (S) at position 314, according to EU numbering.

In one aspect, a Fc domain variant polypeptide comprises an aspartic acid (D) at amino acid position 268, a tyrosine (Y) at amino acid position 330, a valine (V) at position 376, according to EU numbering.

In one aspect, a Fc domain variant polypeptide comprises a glutamic acid (E) at amino acid position 268, a threonine (T) at amino acid position 314, a valine (V) at position 376, according to EU numbering.

In one aspect, a Fc domain variant polypeptide comprises an aspartic acid (E) at amino acid position 268, a valine (V) at amino acid position 314, and a methionine (M) at amino acid position 330, according to EU numbering.

pH-Dependent Fc Domain Variant Polypeptide

The neonatal Fc receptor (FcRn) interacts with the Fc region of antibodies to promote recycling through rescue of normal lysosomal degradation. This process is a pH-dependent process that occurs in the endosomes at acidic pH (e.g., a pH less than 6.5) but not under the physiological pH conditions of the bloodstream (e.g., a non-acidic pH).

In one aspect, a Fc domain variant polypeptide has enhanced FcRn binding affinity at an acidic pH compared to a Fc domain parent polypeptide. In one aspect, a Fc domain variant polypeptide has enhanced FcRn binding affinity at pH less than 7, e.g., at about pH 6.5, at about pH 6.0, at about pH 5.5, at about pH 5.0, compared to a Fc domain parent polypeptide. In another aspect, a Fc domain variant polypeptide has enhanced FcRn binding affinity at pH less than 7, e.g., at about pH 6.5, at about pH 6.0, at about pH 5.5, at about pH 5.0, compared to the FcRn binding affinity of a Fc domain parent polypeptide at an elevated non-acidic pH. An elevated non-acidic pH can be, e.g., pH greater than 7, about pH 7, about pH 7.4, about pH 7.6, about pH 7.8, about pH 8.0, about pH 8.5, about pH 9.0.

In one aspect, it may be desired for a Fc domain variant polypeptide to exhibit approximately the same FcRn binding affinity at non-acidic pH as a Fc domain parent polypeptide. In another aspect, it may be desired for a Fc domain variant to exhibit less FcRn binding affinity at non-acidic pH than a binding polypeptide comprising a modified Fc domain having the double amino acid substitution M428L/N434S, according to EU numbering (See U.S. Pat. No. 8,088,376). Accordingly, it may be desired a Fc domain variant polypeptide to exhibit minimal perturbation to pH dependent FcRn binding.

In one aspect, a Fc domain variant polypeptide having enhanced FcRn binding affinity at an acidic pH, has a reduced (i.e., slower) FcRn off-rate as compared to a Fc domain parent polypeptide. In another aspect, a Fc domain variant polypeptide having enhanced FcRn binding affinity at an acidic pH compared to the FcRn binding affinity of the binding polypeptide at an elevated non-acidic pH, has a slower FcRn off-rate at the acidic pH compared to the FcRn off-rate of a Fc domain parent polypeptide at the elevated non-acidic pH.

### Other Fc Domain Variant Polypeptides

Certain aspects include Fc domain variant polypeptides in which at least one amino acid in one or more of the constant region domains has been deleted or otherwise altered to provide desired biochemical characteristics such as reduced or enhanced effector functions, the ability to non-covalently dimerize, enhanced protein expression, increased protein purity, increased ability to localize at the site of a tumor, reduced serum half-life, or increased serum half-life when compared with a whole, unaltered antibody of approximately the same immunogenicity or when compared with a Fc domain parent polypeptide.

In one aspect, a Fc domain variant polypeptide comprises constant regions derived from different antibody isotypes (e.g., constant regions from two or more of a human IgG1, IgG2, IgG3, or IgG4). In another aspect, a Fc domain variant polypeptide comprises a chimeric hinge (i.e., a hinge comprising hinge portions derived from hinge domains of different antibody isotypes, e.g., an upper hinge domain from an IgG4 molecule and an IgG1 middle hinge domain). In another aspect, a Fc domain may be mutated to increase or decrease effector function relative to a Fc domain parent polypeptide using techniques known in the art.

### Antibody

As used herein, the term “antibody” refers to such assemblies (e.g., intact antibody molecules, antibody fragments, or variants thereof) which have significant known specific immunoreactive activity to an antigen of interest (e.g., a CD25 associated antigen or a target protein associated antigen). Antibodies and immunoglobulins comprise light and heavy chains, with or without an interchain covalent linkage between them.

As will be discussed in more detail below, the generic term “antibody” comprises five distinct classes of antibody that can be distinguished biochemically. While all five classes of antibodies are clearly within the scope of the current disclosure, the following discussion will generally be directed to the IgG class of immunoglobulin molecules. With regard to IgG, immunoglobulins comprise two identical light chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

Light chains of immunoglobulin are classified as either kappa or lambda (K, A). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells. In the heavy chain, the amino acid sequences run from an N-terminus at the forked ends of the Y configuration to the C-terminus at the bottom of each chain. Heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin isotype subclasses (e.g., IgG1, IgG2, IgG3, IgG4, IgA1, etc.) confer functional specialization. Modified versions of each of these classes and isotypes are readily discernable to the skilled artisan in view of the instant disclosure and, accordingly, are within the scope of the current disclosure.

Both the light and heavy chains are divided into regions of structural and functional homology. The term “region” refers to a part or portion of an immunoglobulin or antibody chain and includes constant region or variable regions, as well as more discrete parts or portions of said regions. For example, light chain variable regions include “complementarity determining regions” or “CDRs” interspersed among “framework regions” or “FRs,” as defined herein.

In one aspect, an antibody comprises a Fc domain variant polypeptide as described herein. In another aspect, an antibody comprises a Fc domain variant polypeptide comprising at least one substitution or at least two substitutions (e.g., 1, 2, 3, 4, 5, or more) at amino acid positions 251, 267, 268, 298, 314, 330, 339, 373, and 376, according to EU numbering, as compared to a Fc domain parent polypeptide. In another aspect, an antibody comprises a Fc domain variant polypeptide comprising at least two substitutions at amino acid position 251 or 376, according to EU numbering, as compared to a Fc domain parent polypeptide. In another aspect, an antibody comprises a Fc domain variant polypeptide comprising at least two substitutions at amino acid positions 251, 267, 268, 298, 314, 330, 339, 373, and 376, according to EU numbering, as compared to a Fc domain parent polypeptide.

In another aspect, an antibody comprises a Fc domain variant polypeptide comprising at least two amino acid substitutions are selected from: (i) an alanine (A), a histidine (H), an isoleucine (I), a phenylalanine (F), a glutamine (Q), or a tryptophan (W) at amino acid position 251, (ii) an alanine (A) or an aspartic acid (D) at amino acid position 267, (iii) an aspartic acid (D) or a glutamic acid (E) at amino acid position 268, (iv) an alanine (A) at amino acid position 298, (v) a glycine (G), a lysine (K), an asparagine (N), a methionine (M), a serine (S), a threonine (T), a valine (V), a glutamic acid (E), or a tryptophan (W) at amino acid position 314, (vi) a phenylalanine (F), a methionine (M), or a tyrosine (Y) at amino acid position 330, (vii) a threonine (T) at amino acid position 339, (viii) a tryptophan (W) at amino acid position 373, or (ix) a valine (V) at amino acid position 376, according to EU numbering.

### Constant and Variable Domains

The regions of an immunoglobulin heavy or light chain can be defined as “constant” (C) region or “variable” (V) regions, based on the relative lack of sequence variation within the regions of various class members in the case of a “constant region”, or the significant variation within the regions of various class members in the case of a “variable regions.” The terms “constant region” and “variable region” may also be used functionally. In this regard, it will be appreciated that the variable regions of an immunoglobulin or antibody determine antigen recognition and specificity. Conversely, the constant regions of an immunoglobulin or antibody confer important effector functions such as secretion, transplacental mobility, Fc receptor binding, complement binding, and the like. The subunit structures and three-dimensional configurations of the constant regions of the various immunoglobulin classes are known.

The constant and variable regions of immunoglobulin heavy and light chains are folded into domains. The term “domain” refers to a globular region of a heavy or light chain comprising peptide loops (e.g., comprising 3 to 4 peptide loops) stabilized, for example, by β-pleated sheet and/or intrachain disulfide bond. Constant region domains on the light chain of an immunoglobulin are referred to interchangeably as “light chain constant region domains”, “CL regions” or “CL domains.” Constant domains on the heavy chain (e.g., hinge, CH1, CH2 or CH3 domains) are referred to interchangeably as “heavy chain constant region domains”, “CH” region domains or “CH domains”. Variable domains on the light chain are referred to interchangeably as “light chain variable region domains”, VL region domains or “VL domains.” Variable domains on the heavy chain are referred to interchangeably as “heavy chain variable region domains”, “VH region domains” or “VH domains.”

By convention the numbering of the variable constant region domains increases as they become more distal from the antigen binding site or amino-terminus of the immunoglobulin or antibody. The N-terminus of each heavy and light immunoglobulin chain is a variable region and at the C-terminus is a constant region; the CH3 and CL domains actually comprise the carboxy-terminus of the heavy and light chain, respectively. Accordingly, the domains of a light chain immunoglobulin are arranged in a VL-CL orientation, while the domains of the heavy chain are arranged in the VH-CH1-hinge-CH2-CH3 orientation.

The assignment of amino acids to each variable region domain is in accordance with the definitions of Kabat, Sequences of Proteins of Immunological Interest (National Institutes of Health, Bethesda, MD, 1987 and 1991). Kabat also provides a widely used numbering convention (Kabat numbering) in which corresponding residues between different heavy chain variable regions or between different light chain variable regions are assigned the same number. CDRs 1, 2 and 3 of a VL domain are also referred to herein, respectively, as CDR-L1, CDR-L2 and CDR-L3. CDRs 1, 2 and 3 of a VH domain are also referred to herein, respectively, as CDR-H1, CDR-H2 and CDR-H3. If so noted, the assignment of CDRs can be in accordance with IMGT® (Lefranc et al., Developmental & Comparative Immunology 27:55-77; 2003) in lieu of Kabat. Numbering of the heavy chain constant region is via the EU index as set forth in Kabat (Kabat, Sequences of Proteins of Immunological Interest, National Institutes of Health, Bethesda, MD, 1987 and 1991).

In one aspect, a binding moiety that binds to the target protein comprises an antibody or an antigen binding fragment thereof. In one aspect, an antibody or the antigen binding fragment thereof specific for a target protein comprises a variable domain. In one aspect, a variable domain specific for a target protein is operatively linked to a Fc domain variant polypeptide of the disclosure.

### VH Domain and VL Domain

As used herein, the term “VH domain” includes the amino terminal variable domain of an immunoglobulin heavy chain, and the term “VL domain” includes the amino terminal variable domain of an immunoglobulin light chain. In one aspect, a Fc domain variant polypeptide comprises a VH domain. In one aspect, a Fc domain variant polypeptide comprises a VL domain.

As used herein, the term “CH1 domain” includes the first (most amino terminal) constant region domain of an immunoglobulin heavy chain that extends, e.g., from about positions 114-223 in the Kabat numbering system (EU positions 118-215). The CH1 domain is adjacent to the VH domain and amino terminal to the hinge region of an immunoglobulin heavy chain molecule and does not form a part of the Fc region of an immunoglobulin heavy chain. In one aspect, a Fc domain variant polypeptide comprises a CH1 domain.

As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al. J. Immunol. 1998, 161:4083). In one aspect, a Fc domain variant polypeptide comprises a hinge domain.

### CH2 Domain

As used herein, the term “CH2 domain” includes the portion of a heavy chain immunoglobulin molecule that extends, e.g., from about positions 244-360 in the Kabat numbering system (EU positions 231-340). The CH2 domain is unique in that it is not closely paired with another domain. Rather, two N-linked branched carbohydrate chains are interposed between the two CH2 domains of an intact native IgG molecule. In certain aspects, a Fc domain variant polypeptide comprises a CH2 domain (e.g., a human IgG1 molecule or variant thereof).

### CH3 Domain

As used herein, the term “CH3 domain” includes the portion of a heavy chain immunoglobulin molecule that extends approximately 110 residues from N-terminus of the CH2 domain, e.g., from about positions 361-476 of the Kabat numbering system (EU positions 341-445). The CH3 domain typically forms the C-terminal portion of the antibody. In some immunoglobulins, however, additional domains may extend from CH3 domain to form the C-terminal portion of the molecule (e.g., the CH4 domain in the μ chain of IgM and the e chain of IgE). In certain aspects, a Fc domain variant polypeptide comprises a CH3 domain (e.g., a human IgG1 molecule or variant thereof).

### CL Domain

As used herein, the term “CL domain” includes the constant region domain of an immunoglobulin light chain that extends, e.g., from about Kabat position 107A-216. The CL domain is adjacent to the VL domain. In certain aspects, a Fc domain variant polypeptide of the current disclosure comprises a CL domain derived from a kappa light chain (e.g., a human kappa light chain).

As indicated above, the variable regions of an antibody allow it to selectively recognize and specifically bind epitopes on antigens. That is, the VL domain and VH domain of an antibody combine to form the variable region (Fv) that defines a three dimensional antigen binding site. This quaternary antibody structure forms the antigen binding site present at the end of each arm of the Y. More specifically, the antigen binding site is defined by three complementary determining regions (CDRs) on each of the heavy and light chain variable regions. As used herein, the term “antigen binding site” includes a site that specifically binds (immunoreacts with) an antigen (e.g., a cell surface or soluble antigen). The antigen binding site includes an immunoglobulin heavy chain and light chain variable region and the binding site formed by these variable regions determines the specificity of the antibody. An antigen binding site is formed by variable regions that vary from one antibody to another. In certain aspects, an antibody comprising a Fc domain variant polypeptide of the current disclosure comprises at least one antigen binding site. In certain aspects, an antibody comprising a Fc domain variant polypeptide of the current disclosure comprises at least two antigen binding sites.

In certain aspects, the antigen binding domains are not derived from the same immunoglobulin molecule. In this regard, the variable region may be derived from any type of animal that can be induced to mount a humoral response and generate immunoglobulins against the desired antigen. As such, the variable region of a binding protein may be, for example, of mammalian origin e.g., may be human, murine, rat, goat, sheep, non-human primate (such as cynomolgus monkeys, macaques, etc.), lupine, or camelid (e.g., from camels, llamas and related species).

In naturally occurring antibodies, the six CDRs present on each monomeric antibody are short, non-contiguous sequences of amino acids that are specifically positioned to form the antigen binding site as the antibody assumes its three-dimensional configuration in an aqueous environment. The remainder of the heavy and light variable domains show less inter-molecular variability in amino acid sequence and are termed the framework regions. The framework regions largely adopt a R-sheet conformation and the CDRs form loops which connect, and in some cases form part of, the R-sheet structure. Thus, these framework regions act to form a scaffold that provides for positioning the six CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to the immunoreactive antigen epitope.

### CDR and FR

As used herein, the term “complementarity determining region” or “CDR” refers to sequences of amino acids within antibody variable regions, which confer antigen specificity and binding affinity. In general, there are three CDRs in each heavy chain variable region (HCDR1, HCDR2, HCDR3) and three CDRs in each light chain variable region (LCDR1, LCDR2, LCDR3). “Framework regions” or “FR” refer to the non-CDR portions of the variable regions of the heavy and light chains. In general, there are four FRs in each heavy chain variable region (FR-H1, FR-H2, FR-H3, and FR-H4), and four FRs in each light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745. (“Contact” numbering scheme), Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme), and Honegger A and Pluckthun A, “Yet another numbering scheme for immunoglobulin variable domains: an automatic modeling and analysis tool,” J Mol Biol, 2001 Jun. 8; 309(3):657-70, (AHo numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

A “CDR” or “complementarity determining region,” or individual specified CDRs (e.g., “HCDR1,” “HCDR2,” “HCDR3”), of a given antibody or region thereof, such as a variable region thereof, should be understood to encompass a (or the specific) complementarity determining region as defined by any of the known schemes. Likewise, an “FR” or “framework region,” or individual specified FRs (e.g., “FR-H1,” “FR-H2”) of a given antibody or region thereof, such as a variable region thereof, should be understood to encompass a (or the specific) framework region as defined by any of the known schemes. In some instances, the scheme for identification of a particular CDR or FR is specified, such as the CDR as defined by the IMGT, Kabat, Chothia, AbM, or Contact method. In other cases, the particular amino acid sequence of a CDR or FR is given. Unless otherwise specified, all particular CDR amino acid sequences mentioned in the disclosure are IMGT CDRs. However, alternative CDRs defined by other schemes are also encompassed by the present disclosure, such as those determined by abYsis Key Annotation (Website: abysis.org/abysis/sequence_input/key_annotation/key_annotation.cgi).

### Antibody Variant

Exemplary antibody variants and/or specific antibody binding fragments variants can comprise a Fc domain variant polypeptide of the disclosure. As used herein, the term “antibody variant” and “antibody specific binding fragment variant” includes synthetic and engineered forms of antibodies which are altered such that they are not naturally occurring, e.g., antibodies that comprise at least two heavy chain portions but not two complete heavy chains (such as, domain deleted antibodies or minibodies) and multispecific forms of antibodies altered to bind to two or more different antigens or to different epitopes on a single antigen); heavy chain molecules joined to scFv molecules and the like.

### Antigen Binding Fragments

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen binding fragments thereof. In one aspect, a Fc domain variant polypeptide of the disclosure comprises or is complexed with (e.g., fused to) an antibody. In one aspect, a binding polypeptide of the current disclosure comprises an antibody and a Fc domain variant polypeptide.

Other engineered molecules, such as domain specific binding proteins, single domain binding proteins, domain deleted binding proteins, chimeric binding proteins, CDR grafted binding proteins, diabodies, triabodies, tetrabodies, minibodies, immunoglobulin single variable domains (ISVs) (e.g., monovalent ISVs, bivalent ISVs, etc.), small modular immunopharmaceuticals (SMIPs), and shark variable IgNAR domains, are also encompassed within the expression “antigen binding fragment,” as used herein. In one aspect, a Fc domain variant polypeptide of the disclosure comprises or is complexed with (e.g., fused to) an antigen binding fragment. In one aspect, a binding polypeptide of the current disclosure comprises an antigen binding fragment and a Fc domain variant polypeptide.

The term “multispecific antibody” denotes a binding fragment or derivative thereof that combines the antigen binding sites of two or more antibodies within a single molecule. The terms “antigen binding portion”, “antigen binding fragment”, “binding protein” or “binding moiety” and the like, as used herein, include any naturally occurring, enzymatically obtainable, synthetic, or genetically engineered polypeptide or glycoprotein that specifically binds to at least one target antigen to form a complex.

In one aspect, a Fc domain variant polypeptide of the disclosure comprises or is complexed with (e.g., fused to) a multispecific antibody. In one aspect, a binding polypeptide of the current disclosure comprises a multispecific antibody and a Fc domain variant polypeptide.

In certain aspects, a binding moiety can refer to one or more fragments in an antibody binding fragment that retains the ability to specifically bind a target protein. In certain aspects, the term “antigen binding fragment” refers to a polypeptide fragment of a binding protein. In one aspect, a Fc domain variant polypeptide can comprise or can be linked to an antigen binding fragment that specifically binds a target protein.

A binding fragment or derivative thereof can be derived, e.g., from full multispecific binding protein molecules using any suitable standard techniques such as proteolytic digestion or recombinant genetic engineering techniques involving the manipulation and expression of DNA encoding multispecific binding protein, or a binding fragment or derivative thereof variable and (optionally) constant domains. Such DNA is known and/or is readily available from, e.g., commercial sources, DNA libraries (including, e.g., phage-antibody libraries), or can be synthesized. The DNA can be sequenced and manipulated chemically or by using molecular biology techniques, for example, to arrange one or more variable and/or constant domains into a suitable configuration, or to introduce codons, create cysteine residues, modify, add, or delete amino acids, etc.

### Multispecific

As used herein, a “multispecific” binding protein is a binding protein that specifically binds two or more types of antigens or epitopes. A multispecific binding protein that binds two antigens, and/or two different epitopes of different antigens, is also referred to herein as a “bispecific” binding protein. A multispecific binding protein that binds three antigens, and/or three different epitopes, is also referred to herein as a “trispecific” binding protein. Thus, the multispecific binding protein is able to bind two or more different targets simultaneously. A multispecific binding protein can comprise two different antigen binding sites as well as a Fc domain variant polypeptide which can bind to a Fc receptor. Genetic engineering can be used to design, modify, and produce the multispecific binding protein, or a binding fragment or derivative thereof with a desired set of binding properties and effector functions.

A multispecific binding protein can comprise an antibody or an antigen binding fragment thereof (such as, scFv, Fab, Fab′, Fv, F(ab′)₂), a minibody, a diabody, a triabody, a tetrabody, a tandem di-scFv, a tandem tri-scFv, an immunoglobulin single variable domain (ISV), such as, a VHH (including humanized VHH), a camelized VH, a single domain antibody, a domain antibody, or a dAb).

In one aspect, a Fc domain variant polypeptide of the disclosure comprises or is complexed with (e.g., fused to) a multispecific binding protein. In one aspect, a binding polypeptide of the current disclosure comprises a multispecific binding protein and a Fc domain variant polypeptide.

### Fc-Containing Binding Polypeptides

In one aspect, the present disclosure provides an isolated Fc domain variant polypeptide comprising or complexed with (e.g., fused to) at least one binding domain (e.g., at least one binding polypeptide). In another aspect, a binding domain comprises one or more antigen binding domains. The antigen binding domains need not be derived from the same molecule as a Fc domain parent polypeptide. In another aspect, a Fc domain variant is present in an antibody. In one aspect, a binding polypeptide of the current disclosure comprises a binding domain and a Fc domain variant polypeptide.

In one aspect, a Fc domain variant polypeptide is present in an antibody or is complexed with an antibody. Any antibody from any source or species can be employed with a Fc domain variant polypeptide disclosed herein. Suitable antibodies include without limitation, chimeric antibodies, humanized antibodies, or human antibodies. Suitable antibodies include without limitation, full-length antibodies, monoclonal antibodies, polyclonal antibodies, or single-domain antibodies, such as VHH antibodies. In one aspect, a binding polypeptide of the current disclosure comprises an antibody and a Fc domain variant polypeptide.

In one aspect, a Fc domain variant polypeptide disclosed herein may be bound to or complexed with an antigen binding fragment of an antibody. The term “antigen binding fragment” refers to a polypeptide fragment of an immunoglobulin or antibody which binds antigen or competes with intact antibody (i.e., with the intact antibody from which they were derived) for antigen binding (i.e., specific binding). Antigen binding fragments can be produced by recombinant or biochemical methods that are well known in the art. Exemplary antigen binding fragments include a variable fragment (Fv), Fab, Fab′, and F(ab′)₂, a minibody, a diabody, a triabody, a tandem di-scFv, a tandem tri-scFv, an immunoglobulin single variable domain (ISV). In one aspect, a binding polypeptide of the current disclosure comprises at least one antigen binding fragment and a Fc domain variant polypeptide.

The term “Fab” denotes a binding protein or a binding fragment thereof having a molecular weight of about 50,000 Da and antigen binding activity, in which about a half of the N-terminal side of H chain and the entire L chain, among fragments obtained by treating IgG with a protease, papain, are bound together through a disulfide bond. In one aspect, a Fc domain variant polypeptide disclosed herein may be bound to or complexed with a Fab. In one aspect, a binding polypeptide of the current disclosure comprises at least one Fab and a Fc domain variant polypeptide.

The term F(ab′)₂ refers to a binding protein or a binding fragment thereof having a molecular weight of about 100,000 Da and antigen binding activity, which is slightly larger than a Fab bound via a disulfide bond of the hinge region, among fragments obtained by treating IgG with a protease, pepsin. In one aspect, a Fc domain variant polypeptide disclosed herein may be bound to or complexed with a F(ab′)₂. In one aspect, a binding polypeptide of the current disclosure comprises at least one F(ab′)₂ and a Fc domain variant polypeptide.

The term Fab′ refers to a binding protein or a binding fragment having a molecular weight of about 50,000 Da and antigen binding activity, which is obtained by cutting a disulfide bond of the hinge region of the F(ab′)₂. In one aspect, a Fc domain variant polypeptide disclosed herein may be bound to or complexed with a Fab′. In one aspect, a binding polypeptide of the current disclosure comprises at least one Fab′ and a Fc domain variant polypeptide.

In one aspect, a Fc domain variant polypeptide of the current disclosure comprises a single chain variable region sequence (ScFv). Single chain variable region sequences comprise a single polypeptide having one or more antigen binding sites, e.g., a VL domain linked by a flexible linker to a VH domain. ScFv molecules can be constructed in a VH-linker-VL orientation or VL-linker-VH orientation. The flexible hinge that links the VL and VH domains that make up the antigen binding site includes from about 10 to about 50 amino acid residues. Tandem scFvs (e.g., a tandem di-scFv or a tandem tri-scFv) is two or more ScFv connected by a peptide linker. Connecting peptides are known in the art. Binding polypeptides may comprise at least one scFv and/or at least one constant region. In one aspect, a Fc domain variant polypeptide of the current disclosure may comprise at least one scFv linked or fused to a Fc domain variant. In one aspect, a Fc domain variant polypeptide of the current disclosure may comprise a tandem di-scFv or a tandem tri-scFv. In one aspect, a binding polypeptide of the current disclosure comprises one or more ScFv and a Fc domain variant polypeptide.

In one aspect, a Fc domain variant polypeptide of the current disclosure is a multispecific antibody, e.g., a multivalent or a tetravalent antibody which is produced by fusing a DNA sequence encoding an antibody with a ScFv molecule (e.g., an altered ScFv molecule). For example, in one aspect, a Fc domain variant polypeptide sequence is combined such that the ScFv molecule (e.g., an altered ScFv molecule) is linked at its N-terminus or C-terminus to a Fc domain variant via a flexible linker (e.g., a gly/ser linker). In another aspect, a tetravalent antibody can be made by fusing an ScFv molecule to a connecting peptide, which is fused to a Fc domain variant polypeptide to construct an ScFv-Fab tetravalent molecule. In one aspect, a binding polypeptide of the current disclosure comprises a ScFv-Fab tetravalent comprising a Fc domain variant polypeptide.

In one aspect, a Fc domain variant polypeptide of the current disclosure is an altered minibody. An altered minibody of the current disclosure is a dimeric molecule made up of two polypeptide chains each comprising an ScFv molecule which is fused to a Fc domain variant polypeptide via a connecting peptide. Minibodies can be made by constructing an ScFv component and connecting peptide components using methods described in the art (see, e.g., U.S. Pat. No. 5,837,821 or WO 94/09817AI). In another aspect, a tetravalent minibody can be constructed. Tetravalent minibodies can be constructed in the same manner as minibodies, except that two ScFv molecules are linked using a flexible linker. The linked scFv-scFv construct is then joined to a Fc domain variant polypeptide. In one aspect, a binding polypeptide of the current disclosure comprises an altered minibody comprising a Fc domain variant polypeptide.

In another aspect, a Fc domain variant polypeptide of the current disclosure comprises a diabody, triabody, or a tetrabody. Diabodies are dimeric, tetravalent molecules each having a polypeptide similar to scFv molecules, but usually having a short (less than 10, e.g., about 1 to about 5) amino acid residue linker connecting both variable domains, such that the VL and VH domains on the same polypeptide chain cannot interact. Instead, the VL and VH domain of one polypeptide chain interact with the VH and VL domain (respectively) on a second polypeptide chain (see, for example, WO2002002781A1). Reducing the linker length below three residues can force scFv association into trimers (triabodies, −90 kDa) or tetramers (˜120 kDa) depending on linker length, composition and V-domain orientation. A diabody, a triabody, or a tetrabody of the current disclosure can comprise an scFv-like molecule fused to a Fc domain variant. In one aspect, a binding polypeptide of the current disclosure comprises a diabody and a Fc domain variant polypeptide. In one aspect, a binding polypeptide of the current disclosure comprises a triabody and a Fc domain variant polypeptide. In one aspect, a binding polypeptide of the current disclosure comprises a tetrabody and a Fc domain variant polypeptide.

In one aspect, a Fc domain variant polypeptide of the current disclosure is a VHH. The term “VHH” or “VHH antibody” is a type of single domain antibody that comprises variable heavy chain domains devoid of light chains. Similar to conventional VH domains, VHHs contain four FRs and three CDRs. VHHs have advantages over conventional antibodies. As they are about ten times smaller than IgG molecules, properly folded functional VHHs can be produced by in vitro expression while achieving high yield. Furthermore, VHHs are very stable, and resistant to the action of proteases. The properties and production of VHHs have been reviewed by Harmsen and De Haard H J (Appl. Microbiol. Biotechnol. 2007 November, 77(1):13-22). In one aspect, a Fc domain variant polypeptide is present in a VHH or is complexed (e.g., fused to) a VHH. In one aspect, a binding polypeptide of the disclosure comprises a Fc domain variant polypeptide fused with one or more VHHs.

In one aspect, a Fc domain variant polypeptide of the current disclosure comprises an immunoglobulin single variable domain (ISV), such as a domain antibody, a “dAb,” a VHH (including a humanized VHH), a camelized VHH, other single variable domains, or any suitable fragment of any one thereof, fused to an Fc domain variant. In one aspect, a binding polypeptide of the current disclosure comprises an ISV and a Fc domain variant polypeptide.

The term “immunoglobulin single variable domain” (ISV or ISVD), interchangeably used with “single variable domain”, defines immunoglobulin molecules wherein the antigen binding site is present on, and formed by, a single immunoglobulin domain. This sets immunoglobulin single variable domains apart from “conventional” immunoglobulins (e.g., monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e., a total of 6 CDRs will be involved in antigen binding site formation. ISVs of the so-called “V_{H}3 class” (i.e., ISVs with a high degree of sequence homology to human germline sequences of the V_{H}3 class such as DP-47, DP-51 or DP-29) or ISVs belonging to the so-called “VH4 class” (i.e., ISVs with a high degree of sequence homology to human germline sequences of the V_{H}4 class such as DP-78), as for example described in WO 2007/118 670 A1, can be used herein.

ISVs have advantages over conventional antibodies: they are about ten times smaller than IgG molecules, and as a consequence properly folded functional ISVs can be produced by in vitro expression while achieving high yield. Furthermore, ISVs are very stable, and resistant to the action of proteases. The properties and production of ISVs have been reviewed by Harmsen and De Haard H J (Appl. Microbiol. Biotechnol. 2007 November, 77(1):13-22).

ISVs (in particular V_{HH} sequences and partially humanized VHHs) can in particular be characterized by the presence of one or more “Hallmark residues” (as described herein in Table 1 and in subsequent paragraphs describing NANOBODY® immunoglobulin single variable domains) such that the ISV is a NANOBODY® ISV.

Thus, generally, a NANOBODY® ISV (in particular a V_{HH}, including (partially or fully) humanized V_{HH} and camelized V_{H}) can be defined as an amino acid sequence with the (general) structure: FR1-CDR1-FR2-CDR2-FR3-CDR3-FR4 in which FR1 to FR4 refer to framework regions 1 to 4, respectively, and in which CDR1 to CDR3 refer to the complementarity determining regions 1 to 3, respectively, and in which one or more of the Hallmark residues are as further defined in Table 1. In particular, a NANOBODY® ISV (in particular a V_{HH}, including (partially) humanized V_{HH} and camelized VH) can be an amino acid sequence with the (general) structure: FR1-CDR1-FR2-CDR2-FR3-CDR3-FR4 in which FR1 to FR4 refer to framework regions 1 to 4, respectively, and in which CDR1 to CDR3 refer to the complementarity determining regions 1 to 3, respectively, and in which the framework sequences are as further defined herein. More in particular, an ISV can be an amino acid sequence with the (general) structure: FR1-CDR1-FR2-CDR2-FR3-CDR3-FR4 in which FR1 to FR4 refer to framework regions 1 to 4, respectively, and in which CDR1 to CDR3 refer to the complementarity determining regions 1 to 3, respectively.

The term “immunoglobulin single variable domain (ISV)” encompasses a NANOBODY® VHH as described in or WO 08/020079 or WO 09/138519, and thus in an aspect denotes a VHH, a humanized VHH or a camelized VH (such as a camelized human VH) or generally a sequence optimized VHH (such as e.g., optimized for chemical stability and/or solubility, maximum overlap with known human framework regions and maximum expression).

Generally, NANOBODY® immunoglobulin single variable domains (ISVs) (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Thus, generally, a NANOBODY® ISV can be defined as an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a NANOBODY® ISV can be an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a NANOBODY® ISV can be an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which:
one or more of the amino acid residues at positions 11, 37, 44, 45, 47, 83, 84, 103, 104 and 108 according to the Kabat numbering are chosen from the Hallmark residues mentioned in Table 1 below.

TABLE 1
Hallmark Residues in Nanobody ® ISVs.

| Position | Human V_{H}3 | Hallmark Residues |
|---|---|---|
| 11 | L, V; | L, S, V, M, W, F, T, Q, E, A, R, G, K, Y, N, P, I; |
| | predominantly L | preferably L |
| 37 | V, I, F; | F⁽¹⁾, Y, V, L, A, H, S, I, W, C, N, G, D, T, P, |
| | usually V | preferably F⁽¹⁾ or Y |
| 44⁽⁸⁾ | G | E⁽³⁾, Q⁽³⁾, G⁽²⁾, D, A, K, R, L, P, S, V, H, T, N, W, |
| | | M, I; |
| | | preferably G⁽²⁾, E⁽³⁾ or Q⁽³⁾; |
| | | most preferably G⁽²⁾ or Q⁽³⁾. |
| 45⁽⁸⁾ | L | L⁽²⁾, R⁽³⁾, P, H, F, G, Q, S, E, T, Y, C, I, D, V; |
| | | preferably L⁽²⁾ or R⁽³⁾ |
| 47⁽⁸⁾ | W, Y | F⁽¹⁾, L⁽¹⁾ or W⁽²⁾ G, I, S, A, V, M, R, Y, E, P, T, C, |
| | | H, K, Q, N, D; preferably W⁽²⁾, L⁽¹⁾ or F⁽¹⁾ |
| 83 | R or K; | R, K⁽⁵⁾, T, E⁽⁵⁾, Q, N, S, I, V, G, M, L, A, D, Y, H; |
| | usually R | preferably K or R; most preferably K |
| 84 | A, T, D; | P⁽⁵⁾, S, H, L, A, V, I, T, F, D, R, Y, N, Q, G, E; |
| | predominantly A | preferably P |
| 103 | W | W⁽⁴⁾, R⁽⁶⁾, G, S, K, A, M, Y, L, F, T, N, V, Q, P⁽⁶⁾, |
| | | E, C; preferably W |
| 104 | G | G, A, S, T, D, P, N, E, C, L; preferably G |
| 108 | L, M or T; | Q, L⁽⁷⁾, R, P, E, K, S, T, M, A, H; |
| | predominantly L | preferably Q or L⁽⁷⁾ |

Notes:
⁽¹⁾In particular, but not exclusively, in combination with KERE (SEQ ID NO: 1) or KQRE (SEQ ID NO: 2) at positions 43-46.
⁽²⁾Usually as GLEW (SEQ ID NO: 3) at positions 44-47.
⁽³⁾Usually as KERE (SEQ ID NO: 1) or KQRE (SEQ ID NO: 2) at positions 43-46, e.g., as KEREL (SEQ ID NO: 4), KEREF (SEQ ID NO: 5), KQREL(SEQ ID NO: 6), KQREF (SEQ ID NO: 7), KEREG (SEQ ID NO: 8), KQREW (SEQ ID NO: 9) or KQREG (SEQ ID NO: 10) at positions 43-47. Alternatively, also sequences such as TERE (SEQ ID NO: 11) (for example TEREL (SEQ ID NO: 12)), TQRE (SEQ ID NO: 13) (for example TQREL (SEQ ID NO: 14)), KECE (SEQ ID NO: 15) (for example KECEL (SEQ ID NO: 16) or KECER (SEQ ID NO: 17)), KQCE (SEQ ID NO: 18) (for example KQCEL (SEQ ID NO: 19)), RERE (SEQ ID NO: 20) (for example REREG (SEQ ID NO: 21)), RQRE (SEQ ID NO: 22) (for example RQREL (SEQ ID NO: 23), RQREF (SEQ ID NO: 24) or RQREW (SEQ ID NO: 25)), QERE (SEQ ID NO: 26) (for example QEREG (SEQ ID NO: 27)), QQRE (SEQ ID NO: 28), (for example QQREW (SEQ ID NO: 29), QQREL (SEQ ID NO: 30) or QQREF (SEQ ID NO: 31)), KGRE (SEQ ID NO: 32) (for example KGREG (SEQ ID NO: 33)), KDRE (SEQ ID NO: 34) (for example KDREV (SEQ ID NO: 35)) are possible. Some other possible, but less preferred sequences include for example DECKL (SEQ ID NO: 36) and NVCEL (SEQ ID NO: 37).
⁽⁴⁾With both GLEW (SEQ ID NO: 3) at positions 44-47 and KERE (SEQ ID NO: 1) or KQRE (SEQ ID NO: 2) at positions 43-46.
⁽⁵⁾Often as KP or EP at positions 83-84 of naturally occurring V_{HH} domains.
⁽⁶⁾In particular, but not exclusively, in combination with GLEW (SEQ ID NO: 3) at positions 44-47.
⁽⁷⁾With the proviso that when positions 44-47 are GLEW (SEQ ID NO: 3), position 108 is always Q in (non-humanized) V_{HH} sequences that also contain a W at 103.
⁽⁸⁾The GLEW (SEQ ID NO: 3) group also contains GLEW-like (SEQ ID NO: 3) sequences at positions 44-47, such as for example GVEW (SEQ ID NO: 38), EPEW (SEQ ID NO: 39), GLER, (SEQ ID NO: 40) DQEW (SEQ ID NO: 41), DLEW (SEQ ID NO: 42), GIEW (SEQ ID NO: 43), ELEW (SEQ ID NO: 44), GPEW (SEQ ID NO: 45), EWLP (SEQ ID NO: 46), GPER (SEQ ID NO: 47), GLER (SEQ ID NO: 48) and ELEW (SEQ ID NO: 49).

In one aspect, a Fc domain variant polypeptide comprises multispecific or multivalent antibodies comprising one or more variable domain in series on the same polypeptide chain, e.g., tandem variable domain (TVD) polypeptides. Exemplary TVD polypeptides include the “double head” or “Dual-FV” configuration described in U.S. Pat. No. 5,989,830. In the Dual-Fv configuration, the variable domains of two different antibodies are expressed in a tandem orientation on two separate chains (one heavy chain and one light chain), wherein one polypeptide chain has two VH domains in series separated by a peptide linker (VH1-linker-VH2) and the other polypeptide chain consists of complementary VL domains connected in series by a peptide linker (VL1-linker-VL2). In the cross-over double head configuration, the variable domains of two different antibodies are expressed in a tandem orientation on two separate polypeptide chains (one heavy chain and one light chain), wherein one polypeptide chain has two VH domains in series separated by a peptide linker (VH1-linker-VH2) and the other polypeptide chain consists of complementary VL domains connected in series by a peptide linker in the opposite orientation (VL2-linker-VL1). Additional antibody variants based on the “Dual-Fv” format include the Dual-Variable-Domain IgG (DVD-IgG) bispecific antibody (see U.S. Pat. No. 7,612,181 and the TBTI format (see US 2010/0226923 A1). In one aspect, a Fc domain variant polypeptide comprises multi-specific or multivalent antibodies comprising one or more variable domain in series on the same polypeptide chain fused to a Fc domain variant polypeptide. In one aspect, a binding polypeptide of the current disclosure comprises a TVD polypeptide and a Fc domain variant polypeptide.

In one aspect, a Fc domain variant polypeptide comprises a cross-over dual variable domain IgG (CODV-IgG) bispecific antibody based on a “double head” configuration (see US20120251541 A1, which is incorporated by reference herein in its entirety). In one aspect, a binding polypeptide of the current disclosure comprises a CODV-IgG comprising a Fc domain variant polypeptide.

In one aspect, a Fc domain variant polypeptide comprises a CrossMab or a CrossMab-Fab multispecific format (see WO2009080253 and Schaefer, et al., PNAS (2011), 108: 11187-1191). Antibody variants based on the CrossMab format have a crossover of antibody domains within one arm of a bispecific IgG antibody enabling correct chain association. In one aspect, a binding polypeptide of the current disclosure comprises a CrossMab comprising a Fc domain variant polypeptide.

In one aspect, a Fc domain variant polypeptide can comprise a glycosylated effector-competent polypeptide comprising a multispecific antibody in a T cell engager format. A “T cell engager” refers to binding proteins directed to a host's immune system, more specifically the T cells' cytotoxic activity as well as directed to a tumor target protein. In some embodiments, an isolated effector-competent polypeptide comprises a multispecific antibody in an NK cell engagerformat. An “NK cell engager” refers to binding proteins comprising monoclonal antibody fragments targeting activating NK cell receptors, antigen-specific targeting regions, and a Fc region (Gauthier, et al. Cell (2019), 177: 1701-13). In one aspect, a binding polypeptide of the current disclosure comprises a T cell engager comprising a Fc domain variant polypeptide. In another aspect, a binding polypeptide of the current disclosure comprises a NK cell engager comprising a Fc domain variant polypeptide.

A Fc domain variant polypeptide of the present disclosure, comprising a Fc domain variant described herein, can include the CDR sequences or the variable domain sequences of a known “parent” antibody. In some embodiments, the parent antibody and the antibody of the disclosure can share similar or identical sequences except for modifications to the Fc domain as disclosed herein.

In one aspect, a Fc domain variant polypeptide comprises a therapeutic polypeptide. In another aspect, a therapeutic polypeptide may be a receptor, a ligand, or an enzyme. In another aspect, a therapeutic polypeptide may be a clotting factor. In another aspect, a clotting factor is selected from the group consisting of FI, FII, FIII, FIV, FV, FVI, FVII, FVIII, FIX, FX, FXI, FXII, FXIII), VWF, prekallikrein, high-molecular weight kininogen, fibronectin, antithrombin III, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI-1), plasminogen activator inhibitor-2 (PAI2), any zymogen thereof, any active form thereof, and any combination thereof. In another aspect, a therapeutic polypeptide may be a growth factor. The growth factor can be selected from any growth factor known in the art. In another aspect, the growth factor is a hormone, in one aspect, the growth factor is a cytokine. In another aspect, a growth factor is a chemokine. In another aspect, a binding polypeptide comprises a therapeutic molecule or therapeutic polypeptide linked to the N-terminus and/or the C-terminus of the Fc domain variant polypeptide described herein. In one aspect, the Fc domain variant polypeptide is a Fc-fusion polypeptide.

### Fc Receptor Binding Moieties

In general, a Fc domain variant polypeptide as described herein can function by binding to a Fc receptor and a target protein with high affinity or avidity. In certain aspects, a Fc domain variant polypeptide can bind Fc receptor and/or a target protein with a K_{D} of less than about 1 μM as measured by surface plasmon resonance (e.g., at 25° C. or at 37° C.). In certain aspects, a Fc domain variant polypeptide bind Fc receptor and/or the target protein with a K_{D} of less than about 40 nM, less than about 30 nM, less than about 20 nM, less than about 10 nM less than about 5 nM, less than about 2 nM or less than about 1 nM, as measured by surface plasmon resonance.

In certain aspects, a Fc domain variant polypeptide described herein bind a Fc receptor with a dissociative half-life (t %) of greater than about 1.1 minutes as measured by surface plasmon resonance at, e.g., about 25° C. or 37° C. In certain aspects, a Fc domain variant polypeptide bind a Fc receptor and the target protein with a t % of greater than about 5 minutes, greater than about 10 minutes, greater than about 30 minutes, greater than about 50 minutes, greater than about 60 minutes, greater than about 70 minutes, greater than about 80 minutes, greater than about 90 minutes, greater than about 100 minutes, greater than about 200 minutes, greater than about 300 minutes, greater than about 400 minutes, greater than about 500 minutes, greater than about 600 minutes, greater than about 700 minutes, greater than about 800 minutes, greater than about 900 minutes, greater than about 1000 minutes, or greater than about 1200 minutes, as measured by surface plasmon resonance at 25° C. or 37° C.

In certain aspects, a Fc domain variant polypeptide binds to a Fc receptor on the surface of a cell with an affinity from about 100 pM to about 1 μM (e.g., about 100 pM to about 1,000 pM, about 1,000 pM to about 0.01 μM, about 0.01 μM to about 0.1 μM, or about 0.1 μM to about 1.0 μM). In certain aspects, a Fc domain variant polypeptide binds to the target protein with an affinity from about 100 pM to about 1 μM (e.g., about 100 pM to about 1,000 pM, about 1,000 pM to about 0.01 μM, about 0.01 μM to about 0.1 μM, or about 0.1 μM to about 1.0 μM).

### Glycoengineering

As used herein, the term “glycoengineering” refers to any method for altering the glycoform profile of Fc domain polypeptides (Fc domain variant or Fc domain parental polypeptides) to generate a “modified glycan.” In certain embodiments, glycoengineered Fc domain polypeptides and/or binding proteins comprising Fc domains and methods of making glycoengineered binding proteins are provided or incorporated herein by reference to WO2014164503A1, WO2015143091A1, WO2016057769A2, and U.S. Prov. Patent Application Ser. No. 63/419,188.

In one aspect, a Fc domain variant polypeptide is glycoengineered. In another aspect, a Fc domain variant polypeptide comprises a modified glycan. In one aspect, a Fc domain variant polypeptide is about 20%, 30%, 40%, 50%, 60%, 70%, 90% or more afucosylated. In one aspect, a Fc domain variant polypeptide comprises substantially no fucose.

In another aspect, a Fc domain variant polypeptide comprises an altered number of fucose residues compared to a Fc domain parent polypeptide. In another aspect, a Fc domain variant polypeptide comprises a reduced number of fucose residues relative to a Fc domain parent polypeptide. For example, a Fc domain variant polypeptide can have a reduced number of fucose residues on an N-glycan at Asn297 of the Fc region, i.e., is afucosylated. Afucosylation can increase FcγRII binding on the NK cells and potently increases ADCC.

### Glycosylated Fc Domain Variant Polypeptide

In certain aspects, a Fc domain polypeptide (either a Fc domain variant or a Fc domain parent polypeptide) is glycosylated. Glycosylation of antibodies at conserved positions in their constant regions can have a profound effect on antibody function, particularly effector functioning such as those described above, see for example, Boyd et al (Mol. Immunol, 32: 1311-1318, 1996). Glycosylation of a Fc domain variant described herein wherein one or more carbohydrate moiety is added, substituted, deleted or modified are contemplated. In some embodiments, the glycosylation of the Fc domain is an N-linked glycosylation. Introduction of an asparagine-X-serine or asparagine-X-threonine motif creates a potential site for enzymatic attachment of carbohydrate moieties and may therefore be used to manipulate the glycosylation of a Fc domain variant polypeptide. In Raju et al. (Biochemistry 40: 8868-8876, 2001) the terminal sialylation of a TNFR-IgG immunoadhesin was increased through a process of re-galactosylation and/or re-sialylation using β-1,4-galactosyltransferace and/or alpha, 2,3 sialyltransferase. Increasing the terminal sialylation is believed to increase the half-life of the immunoglobulin.

Antibodies, in common with most glycoproteins, are typically produced as a mixture of glycoforms. This mixture is particularly apparent when antibodies are produced in eukaryotic cells, particularly mammalian cells. A variety of methods have been developed to manufacture defined glycoforms (see Zhang et al. 2004, Science 303: 371; Sears et al, 2001, Science 291: 2344; Wacker et al., 2002, Science 298: 1790; Davis et al. 2002, Chem. Rev. 102: 579; Hang et al., 2001, Acc. Chem. Res. 34: 727). In one aspect, a glycosylated Fc domain comprises a native glycan at amino acid position 297, according to EU numbering. Glycosylation of the asparagine at amino acid position 297 in the CH2 domain of IgG1 can facilitate interaction between the Fc domain and FcγR. Elimination of this glycosylation site eliminates effector function (Leabman, et al., 2013, MAbs 5:896-903). In another aspect, a Fc domain variant polypeptide comprises wild-type levels, or near wild-type levels, of glycosylation at amino acid position 297, according to EU numbering.

In one aspect, a glycosylated Fc domain variant polypeptide comprises an engineered or non-native glycan. In another aspect, an engineered or non-native glycan is a modified glycan that can be conjugated to a therapeutic molecule (e.g., antibody-drug conjugate).

### Engineered N-Linked Glycosylation

Exemplary aspects of a modified glycan linked to a Fc domain via an engineered glycosylation site present on the Fc domain are described in WO2014043361A1, which is incorporated by reference in its entirety.

In certain aspects, a Fc domain variant polypeptide of the disclosure comprising a Fc domain has an engineered N-linked glycosylation site. In certain aspects, a Fc domain variant polypeptide of the disclosure comprises one or more mutations or glycan modifications to modulate Fc mediated effector function. In certain aspects, a Fc domain variant polypeptide can comprise one or more mutations to modulate serum half-life.

In certain aspects, a Fc domain variant polypeptide of the disclosure comprises mutations or glycan modifications to modulate Fc mediated effector function. In certain aspects, a Fc domain variant polypeptide comprises one or more mutations or glycan modifications to modulate serum half-life.

### Oligomannose-Type N-Glycans

Exemplary aspects of a modified glycan linked to a Fc domain variant polypeptide comprising an oligomannose glycan and methods for making the same are described in U.S. Prov. Patent Application Ser. No. 63/419,188. which is incorporated by reference in its entirety,

As used herein, the term “oligomannose-Type N-glycans” or “oligomannose glycans” refers to any one or a combination of the following mannose-rich structures Man₅(GlcNAc)₂, Man₆(GlcNAc)₂, Man₇(GlcNAc)₂, Man₈(GlcNAc)₂, and Man₉(GlcNAc)₂ (See Schachter et al. “Mannose Oligosaccharide”, 4.06.3.3.1, Comprehensive Glycoscience, 2007.) As used herein, Man₅ refers to the structure Man₅(GlcNAc)₂; Man₆ refers to the structure Man₆(GlcNAc)₂; Man₇ refers to the structure Man₇(GlcNAc)₂; Man₈ refers to the structure Man₈(GlcNAc)₂; and Man₉ refers to the structure Man₉(GlcNAc)₂. In certain aspects, the oligomannose glycan is Man₈(GlcNAc)₂ or Man₉(GlcNAc)₂.

In one aspect, a Fc domain variant polypeptide of the disclosure can contain a modified glycan. In another aspect, the modified glycan is a mannose. In another aspect, the mannose is an oligomannose-type N-linked glycan. In another aspect, an oligomannose-type N-glycan comprise an oligosaccharide selected from the group consisting of Man₉(GlcNAc)₂, Man₈(GlcNAc)₂, Man₇(GlcNAc)₂, Man₆(GlcNAc)₂, and Man₅(GlcNAc)₂.

In one aspect, a Fc domain variant polypeptide of the disclosure comprises 20%, 30%, 40%, 50%, 60%, 70%, 90% or more Man₅₋₉(GlcNAc)₂ glycans by molar ratio relative to all N-glycans.

In one aspect, a Fc domain variant polypeptide of the disclosure comprises greater than 70%, 75%, 80%, 85%, 90%, or 95% Man₅₋₉(GlcNAc)₂ N-glycans by molar ratio relative to all N-glycans.

In one aspect, a Fc domain variant polypeptide of the disclosure comprises Man₈ and Man₉ together as the major species of Man₅₋₉(GlcNAc)₂ N-glycans.

In one aspect, a Fc domain variant polypeptide of the disclosure comprises at least 97% Man₅₋₉(GlcNAc)₂ N-glycans by molar ratio relative to all N-glycans.

In one aspect, a Fc domain variant polypeptide of the disclosure comprises less than 30%, 20%, 10%, 5%, 1% of Man₅₋₉(GlcNAc)₂ glycans by molar ratio relative to all N-glycans, or substantially no Man₅₋₉(GlcNAc)₂ glycans by molar ratio relative to all N-glycans.

In one aspect, a Fc domain variant polypeptide of the disclosure comprises a cysteine (C) at amino acid position 292 and a cysteine (C) at amino acid position 302.

### Kifunensine

In one aspect, disclosed herein are Fc domain variant polypeptides that comprise oligomannose-type N-glycans. In another aspect, oligomannose-type N-glycans are the result of culturing cells engineered to express binding polypeptides in the presence of a mannosidase inhibitor (e.g., the α-mannosidase I inhibitor kifunensine or its derivatives or functional homologs). In another aspect, the treatment of cells with the mannosidase inhibitor results in the production of Fc domain variant polypeptides (e.g., an antibody or an antigen fragment thereof comprising a Fc domain variant) carrying oligomannose-type N-glycans while the formation of complex-type N-glycans is prevented.

A cell engineered to express a Fc domain variant polypeptide may be deficient in one or more glycosidases required for early-stage processing of N-glycans. In certain aspects, the culture conditions may be such that the activity of one or more of these glycosidases is inhibited. As a result of one or both of these conditions, oligosaccharide synthesis is shifted toward oligomannose-type species. For example, the cell may be deficient in one or more glycosidases selected from the group consisting of α-glucosidase 1, α-glucosidase 1l, and α-mannosidase 1. Cells deficient in a glycosidase of interest can be engineered using methods as described in, e.g, Tymms, et al. Gene Knockout Protocols (Methods in Molecular Biology), Humana Press, 1st ed., 2001; and in Joyner, Gene Targeting: A Practical Approach, Oxford University Press, 2nd ed., 2000). For instance, glycosidase-deficient cells can be engineered using lectin selection. See Stanley et al. (1975) Biochemistry, 72(9):3323-3327.

In certain aspects, a Fc domain variant polypeptide comprising oligomannose-type N-glycans may be produced by chemical linking of an unglycosylated Fc domain variant polypeptide of an antibody or Fc fusion protein comprising a Fc domain variant and a separately synthesized oligosaccharide moiety.

In another aspect, cells can be engineered to not express one or more glycosidases selected from the group consisting of α-glucosidase 1, α-glucosidase 1l, and α-mannosidase 1. A glycosidase gene can be disrupted by targeted mutagenesis for example, targeting a CRISPR (clustered regularly interspaced short palindromic repeats) site in the glycosidase gene. In one aspect, one or more expression vectors encoding at least a targeting RNA and a polynucleotide sequence encoding a CRISPR-associated nuclease, such as Cas9 is used to engineer a cell to not express a glycosidase gene.

In still other aspects, a cell engineered to express a Fc domain variant polypeptide may be contacted with an inhibitor of one or more glycosidases selected from the group consisting of α-glucosidase I, α-glucosidase II, and α-mannosidase I. In some aspects, inhibitors of these enzymes may be, for example, small molecules or small interfering RNAs (siRNAs). siRNAs are short (20-25 nt) double stranded RNAs that inhibit a glycosidase of interest via post-transcriptional gene silencing. A glycosidase-specific siRNA may be prepared and used as described in U.S. Pat. No. 6,506,559 and/or using other suitable methods (see Appasani, RNA Interference Technology From Basic Science to Drug Development, Cambridge University Press, 1st ed., 2005; and Uei-Ti et al. (2004) Nucleic Acids Res., 32(3):936-948). Examples of small molecule α-glucosidase I inhibitors include castanospermine (see Pan et al. (1983) Biochemistry, 22:3975-3984), deoxynojirimycin (see DNJ; Hettkamp et al. (1984) Eur. J. Biochem., 142:85-90) and N-alkyl and N-alkenyl derivatives thereof (e.g., N-butyl-DNJ); 2,5-dihydromethil-3,4-dihydroxypyrrolidine (see DMDP; Elbein et al. (1984) J. Biol. Chem., 259:12409-12413); and australine (see Molyneux et al. (1988) J. Nat. Prod., 51:1198-1206). Examples of small molecule α-glucosidase II inhibitors include DNJ and N-alkyl and N-alkenyl derivatives thereof; and MDL 25637 (see Hettkamp et al. (1984) Eur. J. Biochem., 142: 85-90; and Kaushal et al. (1988) J. Biol. Chem., 263: 17278-17283). Examples of small molecule α-mannosidase I inhibitors include deoxymannojirimycin (DMJ) (See Legler et al. (1984) Carbohydr. Res., 128:61-72) and derivatives thereof (e.g., N-methyl derivative as described in Bosch et al. (1985) Virology, 143:342-346), 1,4-dideoxy-1,4-imino-D-mannitol (DIM) (See Fleet et al. (1984) J. Chem. Soc. Chem. Commun., 1240-1241 and Palmarzyk et al. (1985) Arch. Biochem. Biophys., 243:35-45), and kifunensine (see Elbein (1990) J. Biol. Chem., 265:15599-15605).

In one aspect, a Fc domain variant polypeptide comprising a modified glycan is produced by culturing cells that express the Fc domain variant polypeptide in the presence of the α-mannosidase I inhibitor, e.g., kifunensine. In another aspect, kifunensine may be used at a concentration of 0.01 to 100 μg/ml, 0.01 to 75 μg/ml, 0.01 to 50 μg/ml 0.01 to 40 μg/ml, 0.01 to 30 μg/ml, 0.01 to 20 μg/ml, 0.1 to 10 μg/ml, 0.1 to 2.0 μg/ml, or 1 to 0.5 μg/ml for a period of at least 12, 24, 48, 72 hours or 4, 7, 10, 20 days or longer, or continuously. In another aspect, CHO or hybridoma cells are incubated with about 0.5-10 μg/ml kifunensine for over 10 days. In another aspect, the kifunensine is used at a concentration of 60 ng/ml to about 2500 ng/ml. In another aspect, the kifunensine is used at a concentration of 2000 ng/ml.

### Oligomannose-Type N-Glycan Binding Polypeptides

In still another aspect, oligomannose-type N-glycans on a Fc domain variant polypeptide disclosed herein comprise one or more oligomannose-type oligosaccharides selected from the group consisting of Man₉(GlcNAc)₂, Man₈(GlcNAc)₂, Man₇(GlcNAc)₂, Man₆(GlcNAc)₂, and Man₅(GlcNAc)₂.

In another aspect, compositions produced by the methods disclosed herein contain at least 20%, 25%, 30%, 35%, 40%, 45%, 50%, 55%, 60%, 65%, 70%, 75%, 80%, 85%, 90%, 95%, or 99% or more (by molar ratio relative to all N-glycans) oligomannose-type glycans Man₅₋₉(GlcNAc)₂. In another aspect, a composition comprising a population of isolated glycosylated Fc domain variant polypeptides each comprising an N-glycan is provided, wherein the composition comprises at least 50% Man₅₋₉(GlcNAc)₂ N-glycans by molar ratio, relative to all N-glycans.

In another aspect, a composition comprising a Fc domain variant polypeptide disclosed herein comprise Man₈ and Man₉ N-glycans as the major species of N-glycans. In another aspect, a composition comprising a population of isolated glycosylated Fc domain variant polypeptide comprising an N-glycan is provided, wherein the composition comprises at least 50% Man₅₋₉(GlcNAc)₂ N-glycans by molar ratio, relative to all N-glycans, and Man₈ and Man₉ containing N-glycans together are the major species.

In one aspect, a Fc domain variant polypeptide disclosed herein contains predominantly Man₉(GlcNAc)₂ N-glycans. In another aspect, a composition comprising a population of isolated glycosylated Fc domain variant polypeptides each comprising a Fc domain variant comprising an N-glycan is provided, wherein the composition comprises at least 40%, 45%, 50%, 55%, 60%, 65%, 70%, 75%, 80%, 85%, 90%, 95%, 96%, 97%, or 99% Man₅₋₉(GlcNAc)₂ N-glycans by molar ratio, relative to all N-glycans. In one aspect, a composition comprising a population of isolated glycosylated Fc domain variant polypeptides comprising an N-glycan is provided, wherein the composition comprises at least 97% Man₅₋₉(GlcNAc)₂ N-glycans by molar ratio, relative to all N-glycans.

In one aspect, a binding polypeptide comprising a Fc domain variant disclosed herein contains diminishing or undetectable amounts of the oligomannose-type N-glycans Man₈(GlcNAc)₂, Man₇(GlcNAc)₂, Man₆(GlcNAc)₂, and Man₅(GlcNAc)₂, while containing minor (e.g., less than 10% relative to all N-glycans) or undetectable amounts of complex type N-glycans (such as, e.g., G0, C1, G2, G0F, G1F, G2F, and G0F-Gn).

In another aspect, the compositions contain less than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% (by molar ratio, relative to all N-glycans) or less Man₅(GlcNAc)₂ and/or Man₆(GlcNAc)₂ N-glycans. In another aspect, the compositions contain minor (i.e., less than 10% by molar ratio relative to all N-glycans) or undetectable amounts of Man₄(GlcNAc)₂. In another aspect, the compositions contain less than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% Man₅₋₉(GlcNAc)₂ glycans (by molar ratio, relative to all N-glycans) or substantially no Man₅₋₉(GlcNAc)₂ by molar ratio, relative to all N-glycans. In another aspect, the compositions contain less than 80%, 75%, 70%, 65%, 60%, 55%, 50%, 45%, 40%, 35% 30%, 25%, 20%, 15%, 10%, 9%, 8%, 7%, 6%, 5%, 4%, 3%, 2%, or 1% (by molar ratio, relative to all N-glycans) or less complex-type glycans.

Glycan composition can be assessed using, e.g., lectin blotting, HPLC and/or mass spectrometry analysis, such as MALDI-TOF (see e.g., Townsend et al. (1997) Techniques in Glybiology, CRC Press).

In one aspect, a Fc domain variant polypeptide carrying oligomannose-type glycans exhibit enhanced ADCC activity as compared to the same Fc domain variant polypeptide produced without the mannosidase inhibitor (e.g., kifunensine) treatment. In another aspect, a Fc domain variant polypeptide carrying oligomannose-type glycans exhibits enhanced binding to a Fc receptor. In one aspect, a Fc domain variant polypeptide carrying oligomannose-type glycans exhibits enhanced binding to a Fcγ receptor. In another aspect, a Fc domain variant polypeptide carrying oligomannose-type glycans exhibits enhanced binding to FcγRIIIa.

In another aspect, a Fc domain variant polypeptide carrying oligomannose-type glycans exhibit substantially same or better binding specificity for the target. In another aspect, a Fc domain variant polypeptide carrying oligomannose-type glycans exhibit substantially the same or higher binding affinity for the target. In another aspect, a Fc domain variant polypeptide carrying oligomannose-type glycans exhibit substantially same or lower binding affinity for mannose receptor.

### Additional Fc Mutations and Functions

In certain aspects, a Fc domain variant polypeptide of the disclosure comprises a modified Fc domain and can further comprise one or more, e.g., two or more, three or more, or four or more, amino acid substitutions that confers a Fc domain variant polypeptide with one or more biochemical characteristics other than glycan modification.

Exemplary modified Fc domain amino acid substitutions that can confer additional biochemical characteristics to Fc domain variant polypeptide described herein are disclosed in WO2021016571A2, which is incorporated by reference in its entirety.

In certain aspects, a modified Fc region of a Fc domain variant polypeptide of the current disclosure comprises one or more mutations to modulate half-life (see e.g., Dall'Acqua et al. (2006) J Biol Chem 281: 23514-24, Zalevsky et al. (2010) Nat Biotechnol 28: 157-9, Hinton et al. (2004) J Biol Chem 279: 6213-6, Hinton et al. (2006) J Immunol 176: 346-56, Shields et al. (2001) J Biol Chem 276: 6591-604, Petkova et al. (2006) Int Immunol 18: 1759-69, Datta-Mannan et al. (2007) Drug Metab Dispos 35: 86-94, Vaccaro et al. (2005) Nat Biotechnol 23: 1283-8, Yeung et al. (2010) Cancer Res 70: 3269-77 and Kim et al. (1999) Eur J Immunol 29: 2819-25. (e.g., T250Q, M252Y, 1253A, S254T, T256E, P2571, T307A, D376V, E380A, M428L, H433K, N434S, N434A, N434H, N434F, H435A and/or H435R).

In certain aspects, a Fc domain variant polypeptide of the current disclosure can have enhanced FcRn binding affinities at both an acidic pH (e.g., less than about 7.0, no more than about 6.5, or no more than about 6.0) and a non-acidic pH (e.g., no less than about 7.0, or no less than about 7.4), as compared to a Fc domain parent polypeptide. In another example, a Fc domain variant polypeptide can comprise one or more amino acid mutations (e.g., substitutions) which alter the effector functions (e.g., ADCC or CDC function) of the Fc domain, as compared to a corresponding Fc domain parent polypeptide, e.g., a molecule having the same structure as the FcRn antagonist except that it has a wild-type Fc domain. In another example, a Fc domain variant polypeptide can comprise a modified Fc domain comprising one or more amino acid mutations (e.g., substitutions) which alter (e.g., increase or decrease) the circulating half-life (e.g., serum half-life) of the FcRn antagonist, as compared to the corresponding Fc domain parent polypeptide.

In certain aspects, a Fc domain variant polypeptide described herein can comprise a modified Fc domain that alters serum half-life compared to a Fc domain parent polypeptide. In certain aspects, a Fc domain variant polypeptide has an increased serum half-life compared to a Fc domain parent polypeptide. In certain aspects, a Fc domain variant polypeptide is modified to alter FcRn binding affinity compared to a Fc domain parent polypeptide. In certain aspects, a modified Fc domain has enhanced FcRn binding affinity compared to a Fc domain parent polypeptide. In certain aspects, a Fc domain variant polypeptide is modified to enhance the FcRn binding affinity at an acidic pH compared to a Fc domain parent polypeptide.

### Fc Domain Variant Polypeptide Expression and Purity

Despite many advances in recombinant and cell culture methodologies, the problem of ensuring proper protein folding can still thwart the most extensive efforts to produce commercially useful amounts of a desired Fc domain variant polypeptide. All Fc domain variant polypeptides must achieve a proper two- and three-dimensional conformation to function while maintaining the ability to modulate a functional attribute, e.g., an effector function as compared to a Fc domain parent polypeptide. Accordingly, the Fc domain variant polypeptides of the disclosure can be assessed for protein expression and purity in addition to modulation of a functional attribute.

Standard techniques may be used to express and purify a Fc domain variant polypeptide of the disclosure. For example, recombinant DNA, oligonucleotide synthesis, transfection of cells (for example, without limitation, electroporation, liposome-mediated transfection, transformation methods), and cell and tissue culture methods can all be used to express a Fc domain variant of the disclosure. Enzymatic reactions and purification techniques may be performed as commonly accomplished as described in a manufacturer's specifications or as described herein. The foregoing techniques and procedures may be performed according to methods as described in various general and more specific references that are cited and discussed throughout the present specification. See e.g., Sambrook et al., eds., Molecular Cloning: A Laboratory Manual, Second Edition (Cold Spring Harbor Laboratory Press, Cold Spring Harbor, New York, 1989) and Ausubel et al. (eds.), Current Protocols in Molecular Biology (John Wiley & Sons, New York, 2012), which are incorporated herein by reference.

In one aspect, a Fc domain variant polypeptide of the disclosure comprises a production titer ranging from about 5, 10, 50, 100, 200, 300, 400 to about 500 milligrams per liter (mg/L). In another aspect, a Fc domain variant polypeptide comprises at least 60, 70, 80, 90, or 100% purity when measured by size exclusion chromatography (SEC).

In one aspect, the disclosure provides a method of producing a Fc domain variant polypeptide comprising performing a Fc domain variant screen wherein Fc domain variant polypeptides are selected if: (a) the Fc domain variant polypeptide has at least a 2-fold increased binding affinity to a Fc receptor relative to the Fc domain parent polypeptide; (b) the Fc domain variant polypeptide production titer is at least 10 mg/L; and (c) the Fc domain variant polypeptide has at least 95% purity when measured by SEC.

### Thermostability

It remains difficult to predict a priori which Fc domain variant polypeptide will suffer from protein instability, e.g., protein unfolding or a propensity form a protein aggregate in response to temperature change. Accordingly, thermostability screening of a Fc domain variant polypeptide can serve as a proxy for assessing conformational stability in vivo as well as under long-term storage conditions. Accordingly, the Fc domain variant polypeptides of the disclosure can be assessed in a thermostability screen in addition to analysis with respect to protein expression, protein purity, and modulation of functional attributes.

Melting temperature (Tm) is a measure of protein thermal stability, where an increase of Tm values reflects the stabilizing effect of selected conditions on protein thermal stability. Methods available to obtain the Tm values include e.g., differential scanning calorimetry (DSC), differential scanning fluorimetry (DSF), or circular dichroism (CD).

In one aspect, a Fc domain variant polypeptide exhibits a Tm greater than 44° C. In another aspect, a Fc domain variant polypeptide exhibits a Tm greater at least 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75° C.

In one aspect, a method of producing the Fc domain variant polypeptide is provided, the method comprising performing a Fc domain variant screen wherein Fc domain variant polypeptides are selected if: (a) the Fc domain variant polypeptide has at least a 2-fold increased binding affinity to a Fc receptor relative to the Fc domain parent polypeptide; (b) the Fc domain variant polypeptide production titer is at least 10 mg/L; and (c) the Fc domain variant polypeptide has at least 95% purity when measured by SEC; and/or (d) the Fc domain variant polypeptide is selected if it exhibits a Tm greater than 44° C.

### Target Proteins

A Fc domain variant polypeptide of the disclosure can comprise a binding moiety which binds a target protein of interest. As used herein a “target protein” can be a protein having a deleterious function and for which degradation can be therapeutically advantageous. In certain aspects, a target protein is a membrane-associated target protein, a soluble target protein, or both. In certain aspects, a target protein is a pathogenic protein or a peptide which causes a disease or symptom of disease. In certain aspects, a target protein is an immune checkpoint protein, a cancer antigen, and/or an immunomodulatory protein. In certain aspects, a target protein is associated with a disease selected from the group consisting of: a cancer, an autoimmune disease, an inflammatory disorder, an infectious disease, and a neurodegenerative disorder.

In other aspects, a target protein is a soluble protein. Exemplary target proteins or peptides include proteins or peptides secreted by tumors, inflammatory protein or peptides; signaling molecules including cytokines, interleukins, interferons, tumor necrosis factors, growth factors, hormones, neurotransmitters, lipid mediators, activating factors, extracellular matrix (ECM) proteins, Wnt proteins, members of the Transforming Growth Factor-beta (TGF-β) family, and Notch ligands. In certain aspects, the target protein is selected from the group consisting of: an antibody, an autoantibody, an inflammatory protein, an interleukin, a cytokine, an interferon, a tumor necrosis factor (TNF), a growth factor, a hormone, a neurotransmitter, a lipid mediator, an activating factor, an extracellular matrix (ECM) protein, a Wnt protein, a member of the Transforming Growth Factor-beta (TGF-β) Family, a Notch ligand, and an immune checkpoint protein.

In certain aspects, a target protein is an antigen. Antigens are molecules that can elicit an immune response. In certain aspects, an antigen is an autoantigen or self-antigen produced in the cell of a subject. For example, an antigen could be a surface marker expressed on specific cell types, allowing a multispecific binding protein of the disclosure to selectively target and modulate those cells. By engaging a CD25⁺ cell via a CD25 binding portion, antigen-targeting multispecific binding proteins of the disclosure can enhance immune responses, facilitate cell-mediated cytotoxicity, or regulate immune cell functions in immunotherapy.

In other aspects, a target protein is an antibody (e.g., an autoantibody) or fragment thereof. An autoantibody is an antibody that specifically binds to one or more antigens made or formed by a subject's own body. Autoantibodies mistakenly recognize and target self-antigens, leading to autoimmune diseases. By binding autoantibodies as the second binding moiety, a multispecific binding protein, or a binding fragment thereof can specifically bind to the self-antigens associated with autoimmune disorders. This approach offers the potential for targeted therapy by redirecting the immune response towards the autoreactive cells or molecules involved in the autoimmune process.

### Disease Indications

In one aspect, the disclosure provides methods of treating a disease or disorder in a subject in need thereof comprising administering to the subject an effective amount of a Fc domain variant polypeptide disclosed herein. In certain embodiments, the present disclosure provides kits and methods for the treatment of diseases and disorders, e.g., cancer in a mammalian subject in need of such treatment.

A Fc domain variant polypeptide of the current disclosure is useful in a number of different applications. For example, in one aspect, a subject Fc domain variant polypeptide is useful for reducing or eliminating cells bearing an epitope recognized by the binding domain of the Fc domain variant. In another aspect, a subject Fc domain variant polypeptide is effective in reducing the concentration of or eliminating soluble antigen in the circulation. In another aspect, a subject Fc domain variant polypeptide is effective as T-cell engagers. In one aspect, a Fc domain variant polypeptide may reduce tumor size, inhibit tumor growth, and/or prolong the survival time of tumor-bearing animals. Accordingly, this disclosure also relates to a method of treating tumors in a human or other animal by administering to such human or animal an effective, non-toxic amount of a Fc domain variant polypeptide.

In another aspect, a subject Fc domain variant polypeptide is useful for the treatment of other disorders, including, without limitation, infectious diseases, autoimmune disorders, inflammatory disorders, lung diseases, neuronal or neurodegenerative diseases, liver diseases, diseases of the spine, diseases of the uterus, depressive disorders and the like. Non-limiting examples of infectious diseases include those caused by RNA viruses (e.g., orthomyxoviruses (e.g., influenza), paramyxoviruses (e.g., respiratory syncytial virus, parainfluenza virus, metapneumovirus), rhabdoviruses (e.g., rabies virus), coronaviruses (e.g., SARS-CoV), alphaviruses (e.g., Chikungunya virus) lentiviruses (e.g., HIV) and the like) or DNA viruses. Examples of infectious diseases also include, without limitation, bacterial infectious diseases, caused by, e.g., Staphylococcus aureus, Staphylococcus epidermidis, Enterococcus, Streptococcus, Escherichia coli, and other infectious diseases including, e.g., those caused by Candida albicans. Other infectious diseases include, without limitation, malaria, SARS, yellow fever, Lyme borreliosis, leishmaniasis, anthrax and meningitis. Exemplary autoimmune disorders include, but are not limited to, psoriasis and lupus. Accordingly, this disclosure relates to a method of treating various conditions that would benefit from using a Fc domain variant polypeptide that has effector modulating functions, e.g., enhanced half-life.

One skilled in the art would be able, by routine experimentation, to determine what an effective, non-toxic amount of Fc domain variant polypeptide would be for the purpose of treating malignancies. For example, a therapeutically active amount of a Fc domain variant polypeptide of the present disclosure may vary according to factors such as the disease stage (e.g., stage I versus stage IV), age, sex, medical complications (e.g., immunosuppressed conditions or diseases) and weight of the subject, and the ability of the modified antibody to elicit a desired response in the subject. The dosage regimen may be adjusted to provide the optimum therapeutic response. For example, several divided doses may be administered daily, or the dose may be proportionally reduced as indicated by the exigencies of the therapeutic situation.

In general, the compositions provided in the current disclosure may be used to prophylactically or therapeutically treat any neoplasm comprising an antigenic marker that allows for the targeting of the cancerous cells by the Fc domain variant.

### Other Terms (Including Polynucleotide/Vector/Host Cell/Pharm Comp) Kabat and EU Numbering

Amino acid positions in a heavy chain constant region, including amino acid positions in the CH1, hinge, CH2, CH3, and CL domains, can be numbered according to the Kabat index numbering system (see Kabat et al., in “Sequences of Proteins of Immunological Interest”, U.S. Dept. Health and Human Services, 5th edition, 1991). Alternatively, antibody amino acid positions can be numbered according to the EU index numbering system. See Kabat et al.

### Specifically Binds

The term “specifically binds” as used herein, refers to the ability of an antibody, an antigen binding fragment thereof, or a Fc domain variant polypeptide to bind to an antigen or to a Fc receptor with a dissociation constant (K_{D}) of at most about 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, or less, and/or to bind to an antigen or a Fc receptor with an affinity that is at least two-fold greater than its affinity for a nonspecific antigen or receptor. Specific binding of an antibody can be to a target antigen through the CDR sequences. An antibody comprising a Fc domain variant polypeptide can also specifically bind to FcRs, such as FcRn or FcγRIIIa through the Fc region.

### Dissociation Constant

The dissociation constant (K_{D}) of a binding protein can be determined, for example, by surface plasmon resonance. Generally, surface plasmon resonance analysis measures real-time binding interactions between ligand (a target antigen on a biosensor matrix) and analyte (a binding protein in solution) by surface plasmon resonance (SPR) using the Biacore system (Cytiva Life Sciences, Marlborough, MA) or Carterra LSA platform (Carterra, Salt Lake City, UT). Surface plasmon analysis can also be performed by immobilizing the analyte (binding protein on a biosensor matrix) and presenting the ligand (target antigen). The term “K_{D}” as used herein refers to the dissociation constant of the interaction between a particular binding protein and a target antigen or a Fc receptor.

### Valency

As used herein the term “valency” refers to the number of potential target binding sites in a polypeptide. Each target binding site specifically binds one target molecule or specific site on a target molecule. When a polypeptide comprises more than one target binding site, each target binding site can specifically bind the same or different molecules (e.g., can bind to more than one target protein, or different epitopes on the same target protein). A subject Fc domain variant polypeptide can have at least one binding site (e.g., 1, 2, 3, or more) specific for a Fc receptor. A subject Fc domain variant polypeptide can have at least one binding site (e.g., 1, 2, 3, 4, or more) for a target protein.

### Specificity

The term “specificity” refers to the ability to specifically bind (e.g., immunoreact with) a given target antigen (e.g., a Fc receptor). In certain aspects, a subject Fc domain variant polypeptide is specific for different types of Fc receptors.

For example, Fc domain variant polypeptide is specific for Fc-gamma receptors (FcγR), Fc-alpha receptors (FcαR), Fc-epsilon receptors (FcεR), and/or neonatal Fc receptor (FcRn). In one aspect, a Fc domain variant polypeptide is specific for FcγR and FcRn but not for FcαR or FcεR. In another aspect, a Fc domain variant polypeptide is specific for FcγRI, FcγRIIa, FcγRIIb, FcγRIIIa, and FcγRIIIb.

In certain aspects, a Fc domain variant polypeptide of the disclosure employ Fc receptor binding moieties which bind to human Fc receptors but not to Fc receptors from other species. Alternatively, a Fc domain variant polypeptide binds to human Fc receptors and to Fc receptors from one or more non-human species. For example, a Fc domain variant polypeptide can bind to human Fc receptor and can bind or not bind, as the case can be, to one or more of mouse, rat, guinea pig, hamster, gerbil, pig, cat, dog, rabbit, goat, sheep, cow, horse, camel, cynomolgus, marmoset, rhesus or chimpanzee Fc receptor. In certain aspects, a Fc domain variant polypeptide can bind to a human Fc receptor, but does not bind to rat and mouse Fc receptor. In other aspects a Fc domain variant polypeptide bind to human Fc receptor, and to rat and mouse Fc receptor, with similar binding affinities.

### About or Approximately

The term “about” or “approximately” means within about 20%, such as within about 10%, within about 5%, or within about 1% or less of a given value or range.

As used herein, “administer” or “administration” refers to the act of injecting or otherwise physically delivering a substance as it exists outside the body (e.g., a Fc domain variant polypeptide provided herein) into a patient, such as by, but not limited to, pulmonary (e.g., inhalation), mucosal (e.g., intranasal), intradermal, intravenous, intramuscular delivery and/or any other method of physical delivery described herein or known in the art. When a disease, or a symptom thereof, is being managed or treated, administration of the substance typically occurs after the onset of the disease or symptoms thereof. When a disease, or symptom thereof, is being prevented, administration of the substance typically occurs before the onset of the disease or symptoms thereof and can be continued chronically to defer or reduce the appearance or magnitude of disease-associated symptoms.

### Composition

As used herein, the term “composition” is intended to encompass a product containing the specified ingredients (e.g., a Fc domain variant polypeptide composition provided herein) in, optionally, the specified amounts, as well as any product which results, directly or indirectly, from combination of the specified ingredients in, optionally, the specified amounts.

### Effective Amount

“Effective amount” means the amount of active pharmaceutical agent (e.g., a Fc domain variant polypeptide of the present disclosure) sufficient to effectuate a desired physiological outcome in an individual in need of the agent. The effective amount can vary among individuals depending on the health and physical condition of the individual to be treated, the taxonomic group of the individuals to be treated, the formulation of the composition, assessment of the individual's medical condition, and other relevant factors.

### Subject or Patient

As used herein, the terms “subject” and “patient” are used interchangeably. As used herein, a subject can be a mammal, such as a non-primate (e.g., cows, pigs, horses, cats, dogs, rats, etc.) or a primate (e.g., monkey and human). In certain aspects, the term “subject,” as used herein, refers to a vertebrate, such as a mammal. Mammals include, without limitation, humans, non-human primates, wild animals, feral animals, farm animals, sport animals, and pets.

### Therapy and Pharmaceutical Compositions

As used herein, the term “therapy” refers to any protocol, method and/or agent that can be used in the prevention, management, treatment and/or amelioration of a disease or a symptom related thereto. In one aspect, the term “therapy” refers to any protocol, method and/or agent that can be used in the modulation or depletion of a target protein from the circulation of a subject. In some aspects, the terms “therapies” and “therapy” refer to a biological therapy, supportive therapy, and/or other therapies useful in the prevention, management, treatment and/or amelioration of a disease or a symptom related thereto, known to one of skill in the art such as medical personnel. In other aspects, the terms “therapies” and “therapy” refer to a biological therapy, supportive therapy, and/or other therapies useful in the modulation of an immune response to an inflammatory or autoimmune diseases in a subject or a symptom related thereto known to one of skill in the art such as medical personnel.

As used herein, the terms “treat,” “treatment” and “treating” refer to the reduction or amelioration of the progression, severity, and/or duration of a disease or a symptom related thereto, resulting from the administration of one or more therapies (including, but not limited to, the administration of one or more prophylactic or therapeutic agents, such as the administration of a Fc domain variant polypeptide or a binding polypeptide comprising a Fc domain variant provided herein). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

Fc domain variant polypeptides e.g., comprised in Fc-containing binding polypeptides of the current disclosure are useful in a number of different applications. In certain aspects, Fc domain variant polypeptides e.g., comprised in Fc-containing binding polypeptides disclosed herein is effective in reducing the concentration of or eliminating a target protein in the circulation. In certain aspects, a Fc domain variant polypeptide e.g., comprised in a Fc-containing binding polypeptide can reduce inflammatory symptoms. In certain aspects, a Fc domain variant polypeptide e.g., comprised in a binding polypeptide of the disclosure can reduce tumor size. Methods of preparing and administering, a Fc domain variant polypeptide e.g., comprised in a binding polypeptide of the current disclosure to a subject are well known to or are readily determined by those skilled in the art.

The route of administration of a Fc domain variant polypeptide of the current disclosure can be oral, parenteral, by inhalation, or topical, or other suitable method. The term parenteral as used herein includes intravenous, intraarterial, intraperitoneal, intramuscular, subcutaneous, rectal, or vaginal administration. While all these forms of administration are clearly contemplated as being within the scope of the current disclosure, a form for administration would be a solution for injection, in particular for intravenous or intraarterial injection or drip. Usually, a suitable pharmaceutical composition for injection can comprise a buffer (e.g., acetate, phosphate or citrate buffer), a surfactant (e.g., polysorbate), optionally a stabilizer agent (e.g., human albumin), etc. However, in other methods compatible with the teachings herein, a Fc domain variant polypeptide can be delivered directly to the site of the adverse tissue thereby increasing the exposure of the diseased tissue to the therapeutic agent.

In certain aspects, a pharmaceutical composition comprises a Fc domain variant polypeptide described herein and a pharmaceutically acceptable carrier or diluent. In certain aspects, a method of depleting a target protein comprises administering to a subject an effective amount of a Fc-containing binding polypeptide comprising a Fc domain variant polypeptide of the disclosure or the pharmaceutical composition comprising the Fc domain variant polypeptide.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the compositions and methods of the current disclosure, pharmaceutically acceptable carriers include, but are not limited to, 0.01-0.1 M, e.g., 0.05M phosphate buffer, or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as for example, antimicrobials, antioxidants, chelating agents, and inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will typically be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

In many cases, isotonic agents will be included, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a Fc domain variant polypeptide or an antibody or antibody fragment thereof comprising a Fc domain variant polypeptide by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, exemplary methods of preparation include vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Such articles of manufacture will typically have labels or package inserts indicating that the associated compositions are useful for treating a subject suffering from or predisposed to autoimmune or neoplastic disorders.

Effective doses of a Fc domain variant polypeptide compositions of the present disclosure, for the treatment of the conditions described above vary depending upon many different factors, including means of administration, target site, physiological state of the patient, whether the patient is human or another animal, other medications administered, and whether treatment is prophylactic or therapeutic. Usually, the patient is a human but non-human mammals including transgenic mammals can also be treated. Treatment dosages can be titrated using routine methods known to those of skill in the art to optimize safety and efficacy.

Fc domain variant polypeptides of the current disclosure can be administered on multiple occasions. Intervals between single dosages can be weekly, monthly or yearly. Intervals can also be irregular as indicated by measuring blood levels of a target protein in the patient. Alternatively, Fc domain variant polypeptide can be administered as a sustained release formulation, in which case less frequent administration is required. For Fc domain variant polypeptides, dosage and frequency vary depending on the half-life of the Fc domain variant polypeptide in the patient.

The dosage and frequency of administration can vary depending on whether the treatment is prophylactic or therapeutic. In prophylactic applications, compositions containing the present a Fc domain variant polypeptide or a cocktail thereof are administered to a patient not already in the disease state to enhance the patient's resistance. Such an amount is defined to be a “prophylactic effective dose.” In this use, the precise amounts again depend upon the patient's state of health and general immunity, but generally range from about 0.1 to about 25 mg per dose, especially about 0.5 to about 2.5 mg per dose. A relatively low dosage is administered at relatively infrequent intervals over a long period of time. Some patients continue to receive treatment for the rest of their lives. In therapeutic applications, a relatively high dosage (e.g., from about 1 to 400 mg/kg of a Fc domain variant polypeptide per dose) at relatively short intervals is sometimes required until progression of the disease is reduced or terminated, or until the patient shows partial or complete amelioration of disease symptoms. Thereafter, the patient can be administered a prophylactic regime.

A pharmaceutical composition in accordance with the present disclosure can comprise a pharmaceutically acceptable, non-toxic, sterile carrier such as physiological saline, nontoxic buffers, preservatives and the like. For the purposes of the instant application, a pharmaceutically effective amount of a Fc domain variant polypeptide disclosed herein, shall be held to mean an amount sufficient to achieve effective binding to a target protein and to achieve a benefit, e.g., to ameliorate symptoms of a disease or disorder.

### Determining Specificity

The binding affinity of a Fc domain variant polypeptide that binds to a Fc receptor (e.g., FcγRIIIa or FcRn) can be assessed using, e.g., surface plasmon resonance, ELISA, or other suitable method (see Shields et al. (2001) J. Biol. Chem., 276:6591-6604).

In one aspect, the binding constant K_{D} of a Fc domain variant polypeptide for a Fc receptor can be above that of a Fc domain parent polypeptide by 1, 2, 3, 4, 5, 6, 7, 8, 9, 10, 11, 12, 13, 14, 15, 16, 17, 18, 19, or 20-fold or higher. The Fc domain parent polypeptide can be an antibody or an antigen binding fragment thereof that comprises a Fc domain (e.g., a wild-type Fc IgG domain). The binding constant K_{D} of a Fc domain variant polypeptide for Fc receptor can be substantially the same (i.e., ±50%) as a Fc domain parent polypeptide or above it. In one aspect, a Fc domain variant polypeptide has about a 1.5-fold to a 20-fold increased binding affinity to a Fc receptor relative to the Fc domain parent polypeptide.

In one aspect, the binding constant K_{D} of a Fc domain variant polypeptide for FcγRIIIa can be above that of the wild-type control by 1, 2, 3, 4, 5, 6, 7, 8, 9, 10, 11, 12, 13, 14, 15, 16, 17, 18, 19, or 20-fold or higher. The wild-type control can be an antibody or an antigen binding fragment thereof that comprises a Fc domain parent polypeptide (e.g., a wild-type Fc IgG domain). The binding constant K_{D} of a Fc domain variant polypeptide for FcγRIIIa can be substantially the same (i.e., ±50%) as a Fc domain parent polypeptide or above it.

In one aspect, the binding constant K_{D} of a Fc domain variant polypeptide for FcRn can be above that of the wild-type control by 1, 2, 3, 4, 5, 6, 7, 8, 9, 10, 11, 12, 13, 14, 15, 16, 17, 18, 19, or 20-fold or higher. The wild-type control can be an antibody or an antigen binding fragment thereof that comprises a Fc domain parent polypeptide (e.g., a wild-type Fc IgG domain). The binding constant K_{D} of a Fc domain variant polypeptide for FcRn can be substantially the same (i.e., ±50%) as a Fc domain parent polypeptide or above it.

In one aspect, the binding constant K_{D} of a Fc domain variant polypeptide of the disclosure for a Fc receptor can be substantially the same (i.e., ±50%) as the wild-type control or below it. In one aspect, the binding constant K_{D} of a Fc domain variant polypeptide of the disclosure for FcγRIIIa can be substantially the same (i.e., ±50%) as the wild-type control or below it. In one aspect, the binding constant K_{D} of a Fc domain variant polypeptide of the disclosure for FcRn can be substantially the same (i.e., ±50%) as the wild-type control or below it.

The binding specificity of a Fc domain variant polypeptide to a Fc receptor or to a FcγRIIIa can be determined by, e.g., flow cytometry, western blotting, or another suitable method. In some aspects, a Fc domain variant polypeptide specifically binds to a Fc receptor (e.g., FcγRIIIa and/or FcRn) and a target protein.

In one aspect, certain pharmacokinetic parameters of a Fc domain variant polypeptide of the disclosure are same or better that those of a Fc domain parent polypeptide (e.g., a wild-type IgG Fc domain) or above it. For example, in one aspect, elimination half-life (t_{1/2}) and/or the area under the concentration curve (AUC) can be substantially the same (i.e., ±50%) as the wild-type control or above it. Pharmacokinetic parameters can be measured in humans or using an appropriate animal model (see, e.g., Shargel et al. (1995) Applied Biopharmaceutics and Pharmacokinetics, 4th ed., McGraw-Hill/Appleton).

### Polynucleotides

In one aspect, polynucleotides (i.e., nucleic acid molecules) encoding a Fc domain variant polypeptide described herein or variants thereof are provided. A polynucleotide variant as used herein is about 50, 75, 80, 85, 90, 93, 95, 98, 99% or more identical to a polynucleotide that encodes a Fc domain variant polypeptide described herein.

Methods of making a Fc domain variant polypeptide comprising expressing these polynucleotides are also provided.

Polynucleotides encoding a Fc domain variant polypeptide disclosed herein are typically inserted in an expression vector for introduction into host cells that can be used to produce the desired quantity. Accordingly, in certain aspects, the disclosure provides expression vectors comprising polynucleotides disclosed herein and host cells comprising these vectors and polynucleotides.

In one aspect, nucleic acid molecules encode an amino acid sequence for a Fc domain variant polypeptide.

### Expression Vector and Host Cell

The term “vector” or “expression vector” is used herein for the purposes of the specification and claims, to mean vectors used in accordance with the present disclosure as a vehicle for introducing into and expressing the polynucleotide sequence encoding a Fc domain variant polypeptide in a cell. As known to those skilled in the art, such vectors can easily be selected from the group consisting of plasmids, phages, viruses and retroviruses. In general, vectors compatible with the instant disclosure will comprise a selection marker, appropriate restriction sites to facilitate cloning of the desired gene and the ability to enter and/or replicate in eukaryotic or prokaryotic cells.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

One or more polynucleotides encoding a Fc domain variant polypeptide can also be expressed in non-mammalian cells such as bacteria or yeast or plant cells. In this regard it will be appreciated that various unicellular non-mammalian microorganisms such as bacteria can also be transformed; i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides can become part of inclusion bodies. The polypeptides must be isolated, purified and then assembled into functional molecules.

In addition to prokaryotes, eukaryotic cells can also be used. Saccharomyces cerevisiae, or common baker's yeast, is the most commonly used although a number of other strains are commonly available.

In some aspect, a vector comprises nucleic acid molecules encoding an amino acid sequence for a Fc domain variant polypeptide.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present disclosure has been described with reference to the specific aspects thereof, it should be understood by those skilled in the art that various changes can be made and equivalents can be substituted without departing from the true spirit and scope of the application. It will be readily apparent to those skilled in the art that other suitable modifications and adaptations of the methods described herein can be made using suitable equivalents without departing from the scope of the aspects disclosed herein. In addition, many modifications can be made to adapt a particular situation, material, composition of matter, process, process step or steps, to the objective, spirit and scope of the present disclosure. All such modifications are intended to be within the scope of the claims appended hereto. Having now described certain aspects in detail, the same will be more clearly understood by reference to the following examples, which are included for purposes of illustration only and are not intended to be limiting.

### EXAMPLES

The present disclosure is further illustrated by the following examples which should not be construed as further limiting. The contents of the Sequence Listing, figures and all references, patents, and published patent applications cited throughout this application are expressly incorporated herein by reference.

### Example 1. Engineering, Expression, and Purification Fc Domain Variants

### Introduction

One hundred twenty-five (125) Fc domain variant polypeptides were designed, each featuring one or more mutations relative to a wild-type Fc domain parent polypeptide. Each mutation introduced was made to change an amino acid residue at the protein coding sequence level. Subsequently, Fc domain variant polypeptides underwent screening on three distinct parameters: (1) protein expression profile; (2) protein purity; and (3) FcγRIIIa binding capability (discussed in Example 2).

### Material and Methods

The designed Fc domain variant polypeptides were expressed and purified using the following methods.

### Expression and Purification

Point mutations were introduced into the nucleic acid sequences encoding the human IgG1 Fc domain. These nucleic acid sequences were then fused to the coding sequence of the variable domain of mAb1 (an IgG1 antibody directed against a protein target of interest and cloned into mammalian expression plasmids containing a cytomegalovirus (CMV) enhancer/promoter and the SV40 polyA signal. The resulting plasmids were transfected into HEK293 cells according to the manufacturer's instructions. Fc domain variant polypeptides were then purified from the transfected HEK293 cell lines using purification techniques according to manufacturer's specifications.

### Results

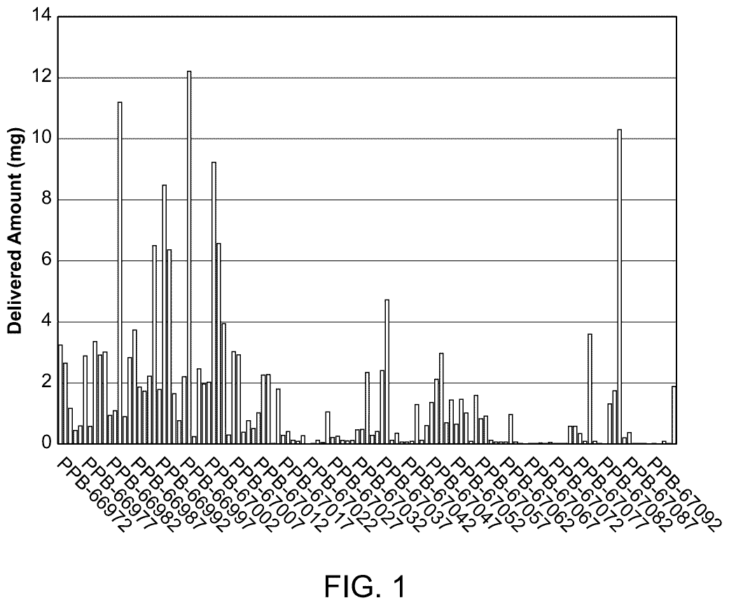

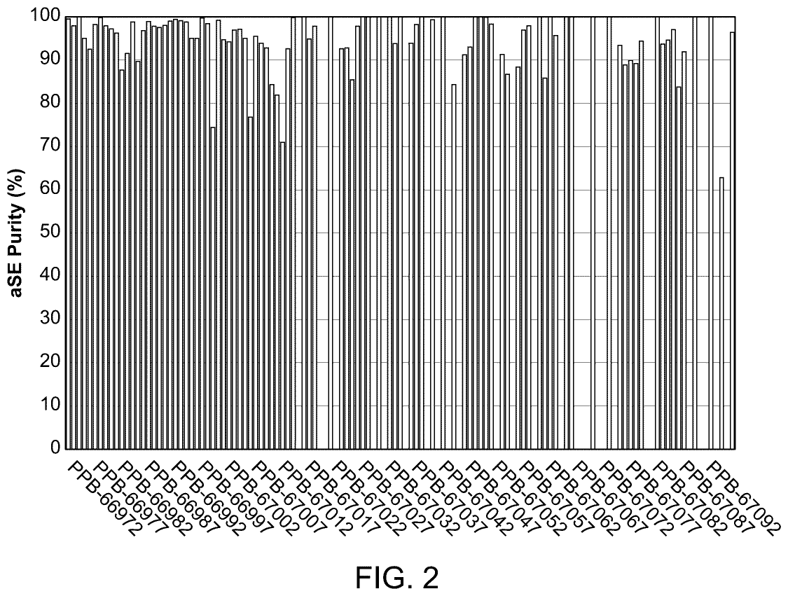

Among the 125 Fc domain variant polypeptides that were expressed, only 47 (approximately 38% of total) exhibited protein production levels greater than 0.2 mg production as illustrated in FIG. 1. In the assessment of protein purity, 36 of the 125 Fc domain variant polypeptides (approximately 29% of total) demonstrated purity levels greater than 80% as shown in FIG. 2.

### Conclusion

The acquisition of expression and purity profiles for all 125 Fc domain variant polypeptides facilitated the exclusion of polypeptides that displayed improper folding and/or formed protein aggregates. Consequently, Fc domain variant polypeptides which exhibited protein expression exceeding 0.2 mg and a minimum purity of 80% were valuable criteria used for selecting Fc domain variant polypeptides for downstream analysis.

### Example 2: Fc Domain Variants with Modulated Fc Function

### Introduction

The 125 Fc domain variant polypeptides described in Example 1 were tested for their ability to enhance FcγR binding (in particular, hFcγRIIIa binding) relative to Fc domain parent polypeptide as well as additional control Fc domain parent polypeptides.

### Methods

Fc Fragments were captured to immobilized protein AG and flow with multiple concentrations of hFcγRIIIa-V158 used to measure binding.

### Sample Preparation

PEPP samples were filtered at 0.22 μM in a 96-well filter plate. A280 was measured on Stunner. Samples were normalized to 100 μg/mL then 10 μg/mL in PBS at pH 7.2 on Hamilton. The sample was diluted to 0.25 μg/mL in 384 well plates with Benchsmart in duplicate.

### Procedure on Carterra LSA

Protein AG was immobilized to a HC30M sensor chip using amine chemistry. Load antibodies were diluted to 0.25 μg/mL per 3 minutes in multiple prints. Multiple concentrations of hFcγRIIIa-V158 were injected for 2 minutes with 5 minutes of dissociation, including twelve 2-fold serial dilutions from 4000 nM to 1.9 nM in HBS-EP+at pH 7.4 (dropped 4 uM and 2 uM concentrations due to avidity). The chips were regenerated with 3×60 seconds of 0.425% Phosphoric Acid with 1 minute stabilization. Finally, the sensorgrams were fit using a 1:1 kinetic binding model.

### Results

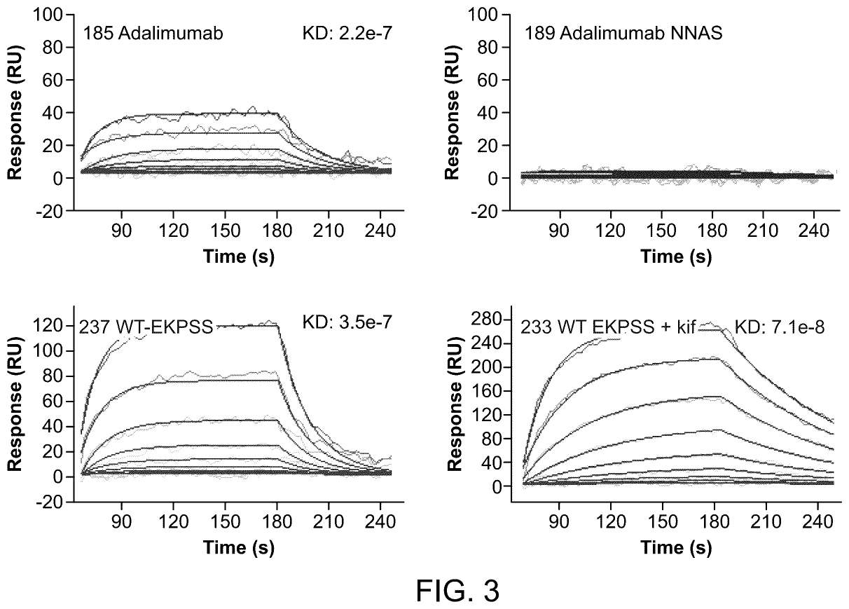

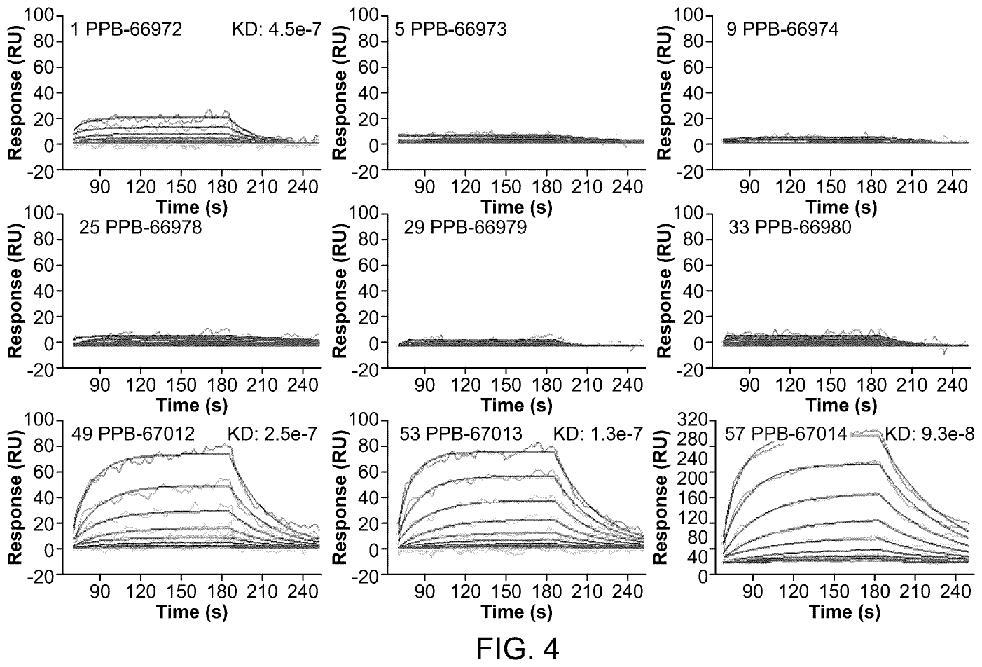

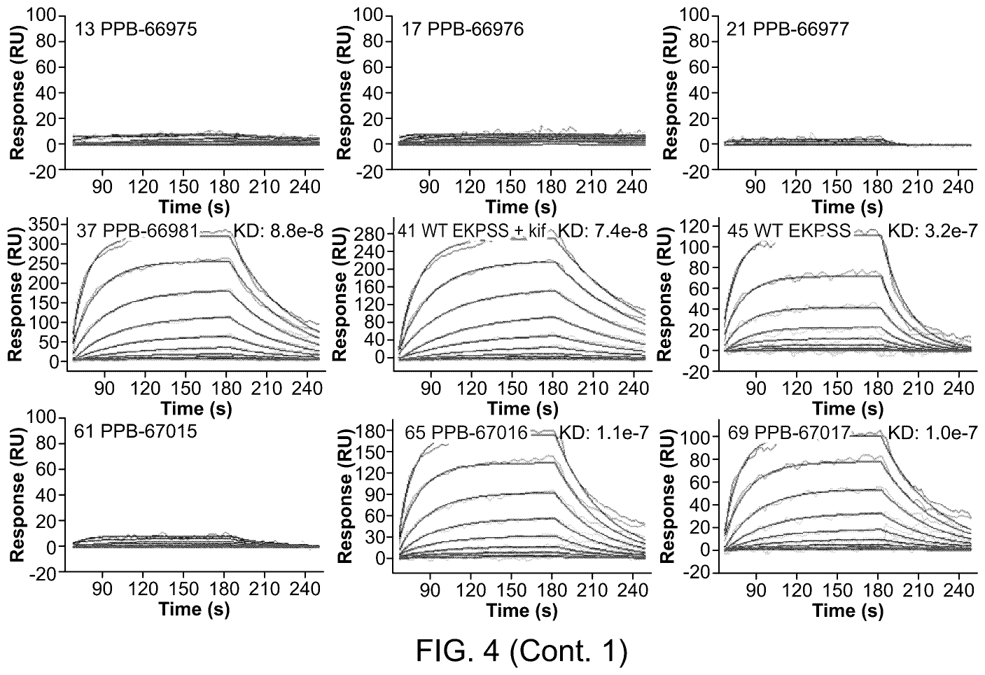

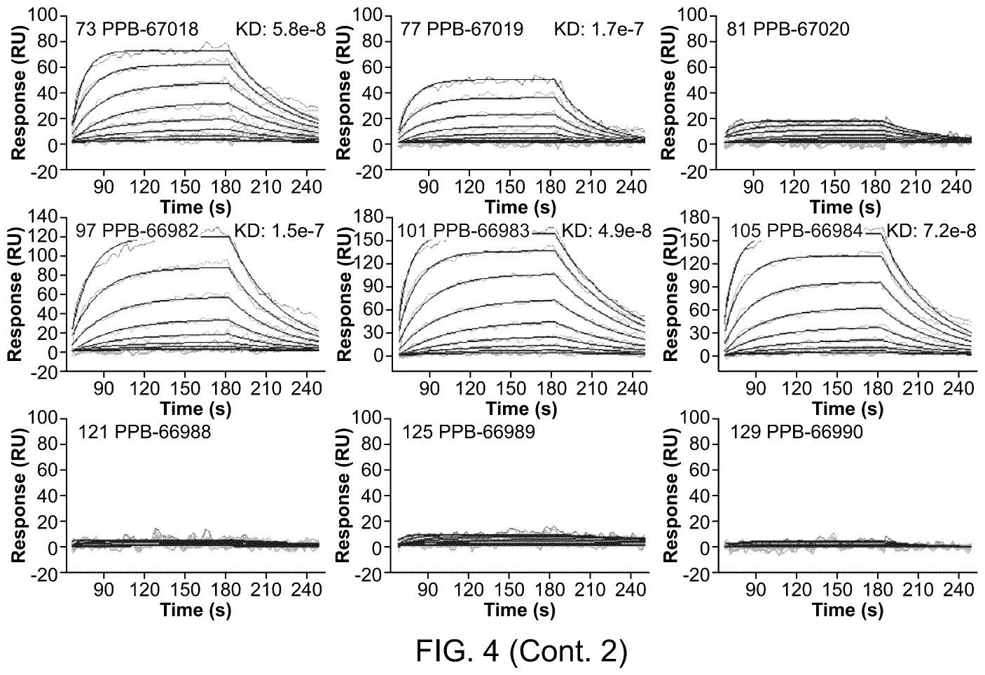

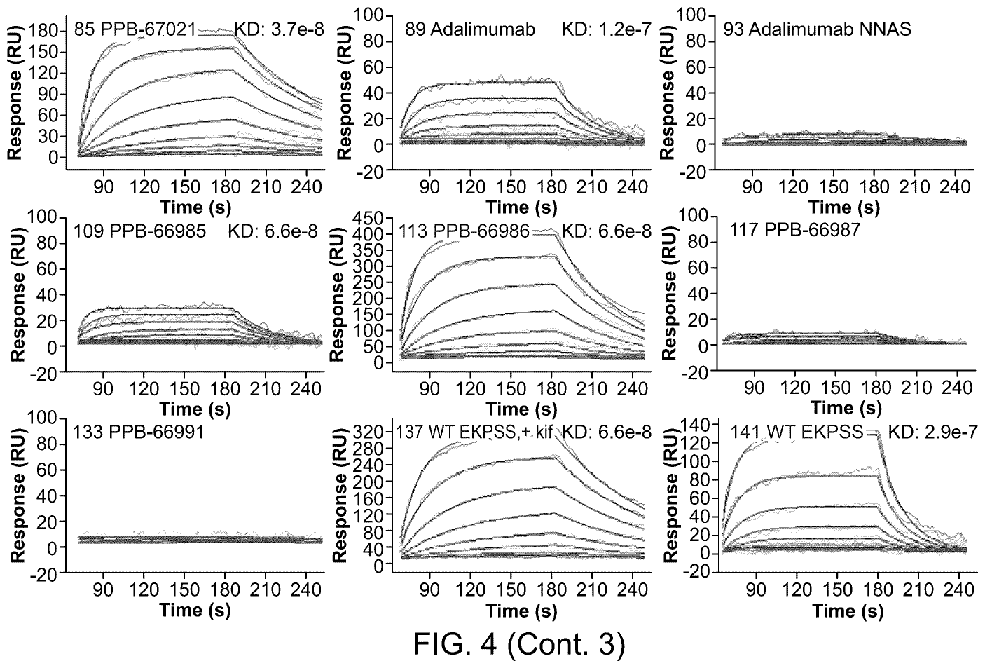

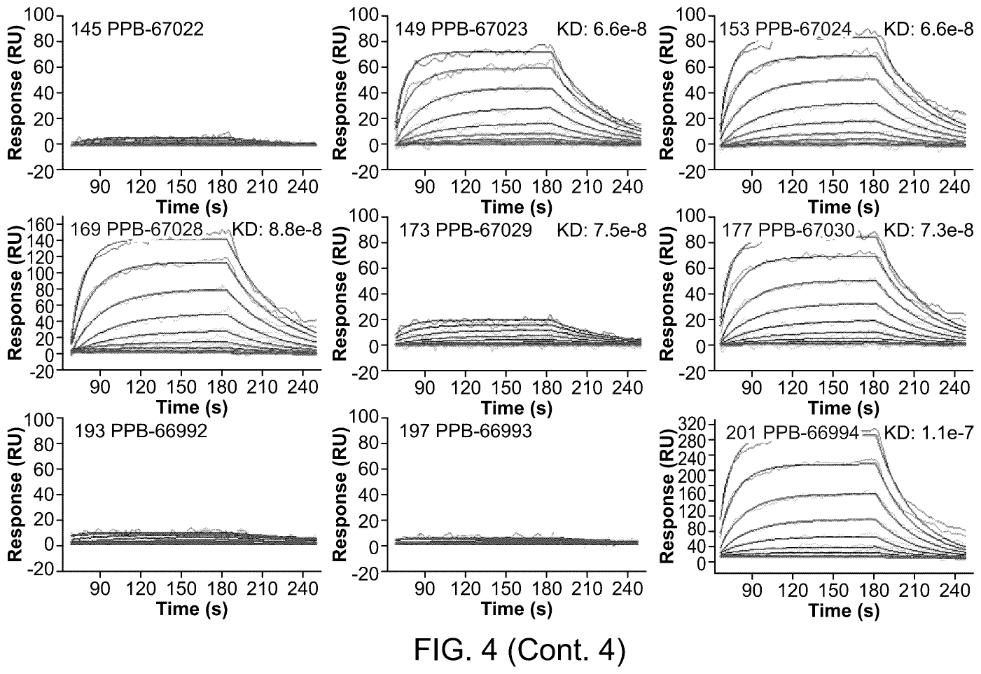

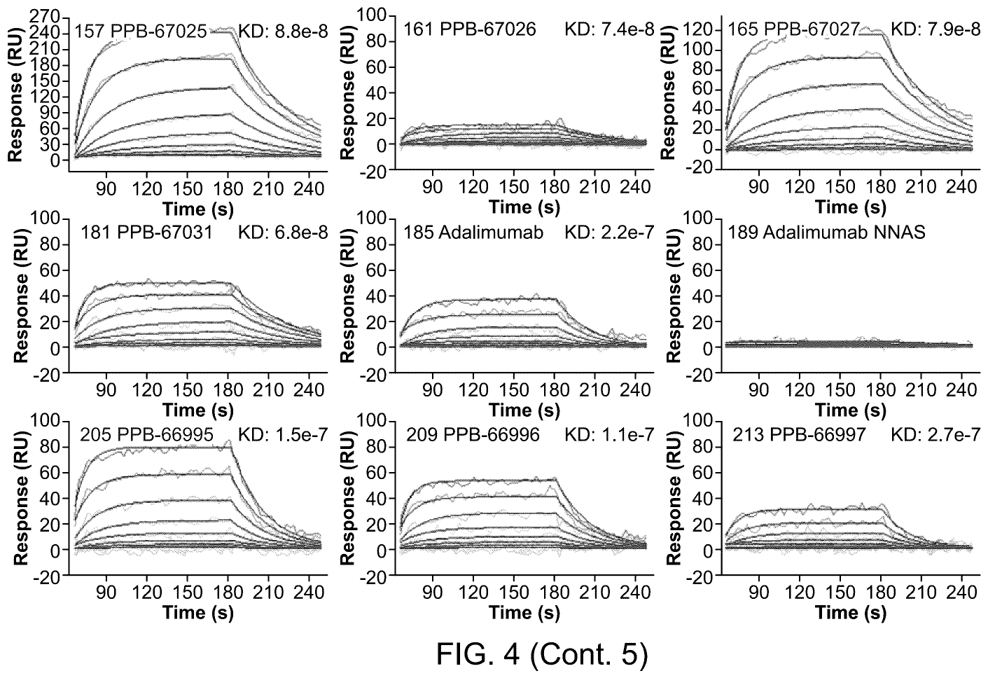

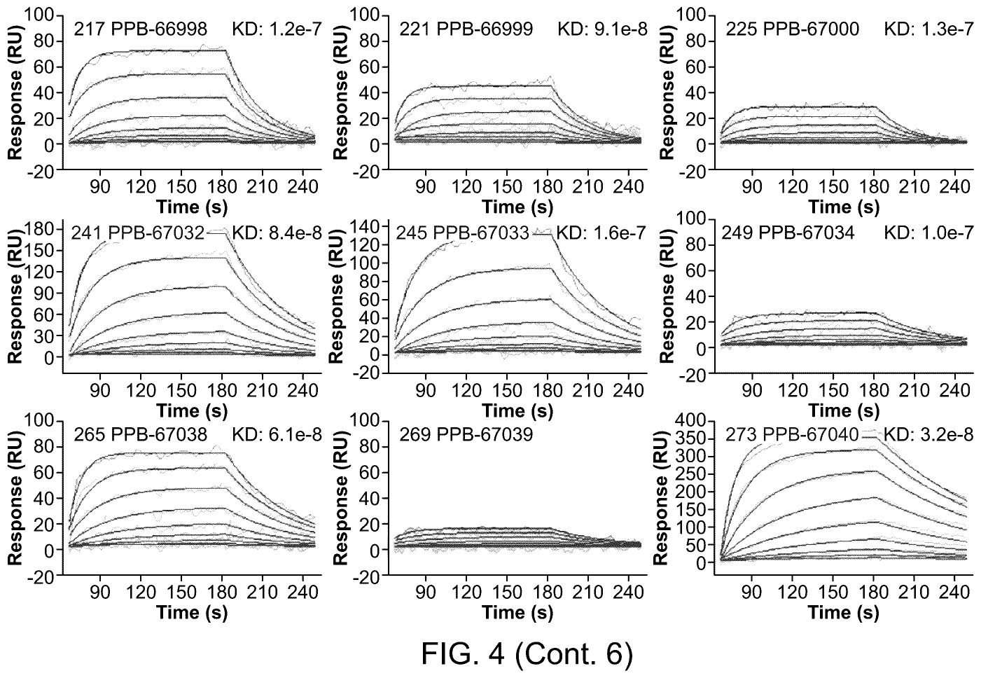

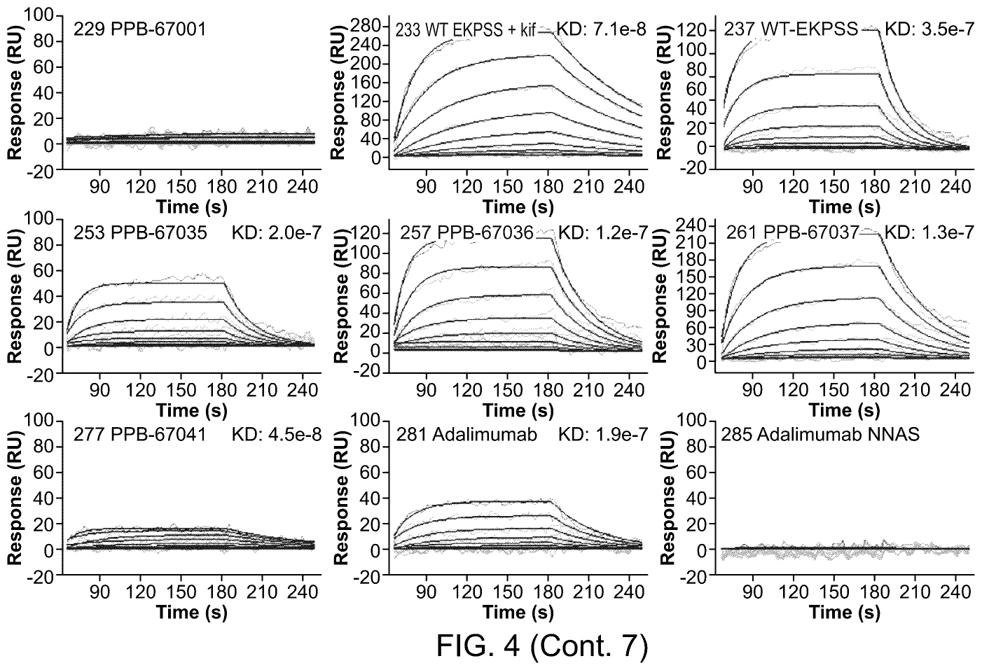

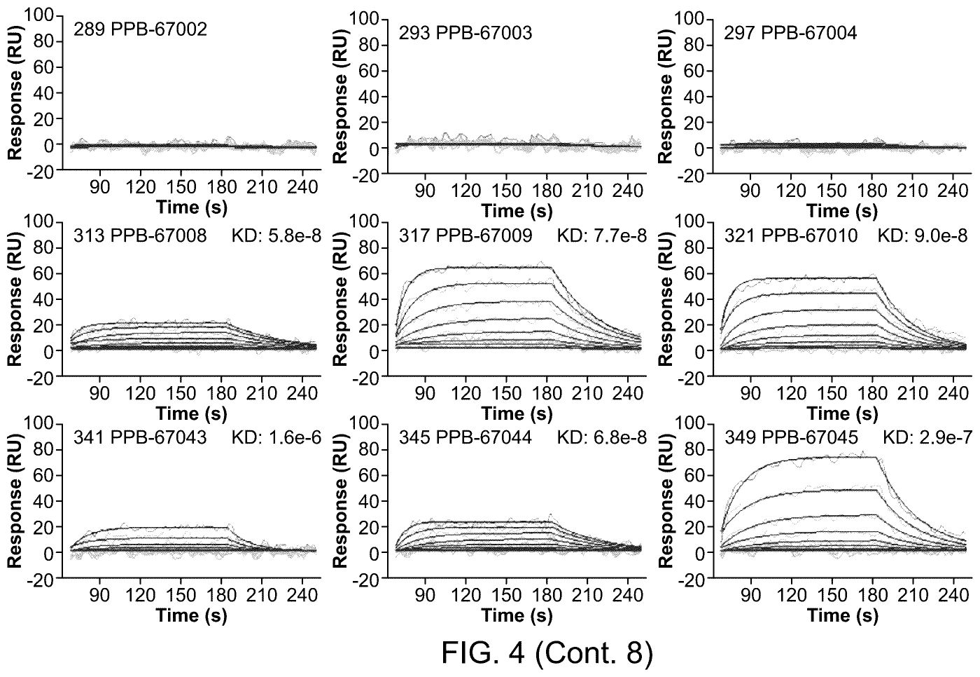

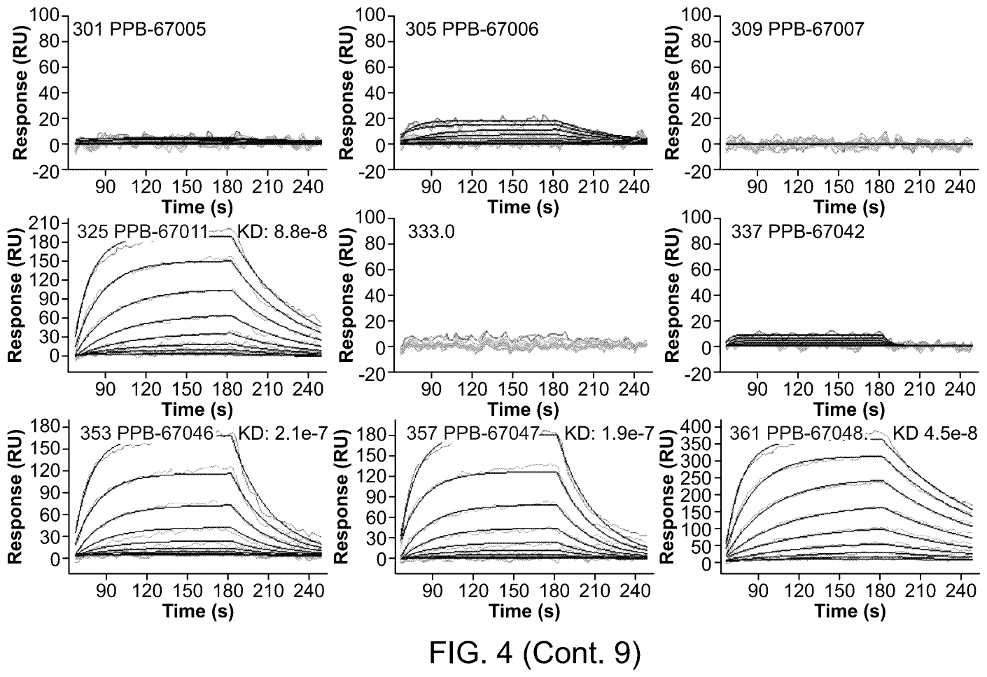

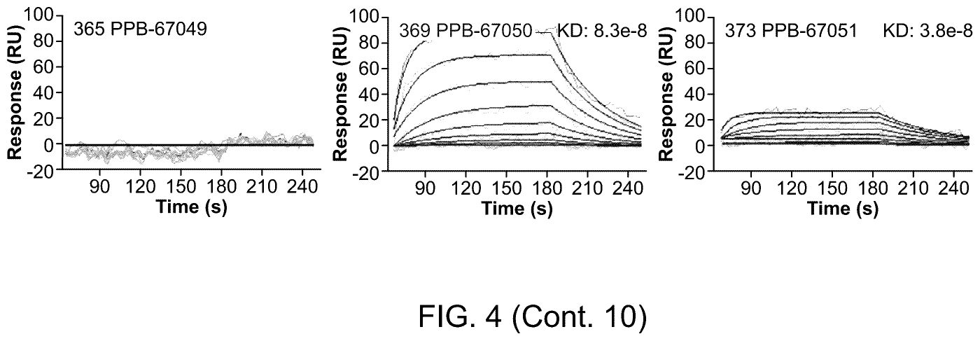

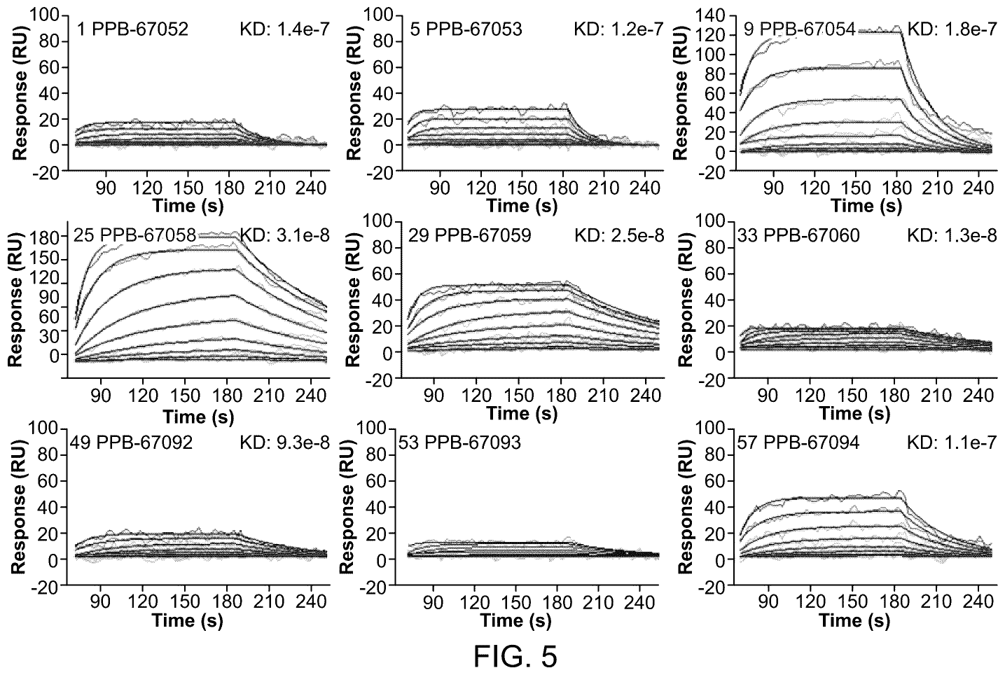

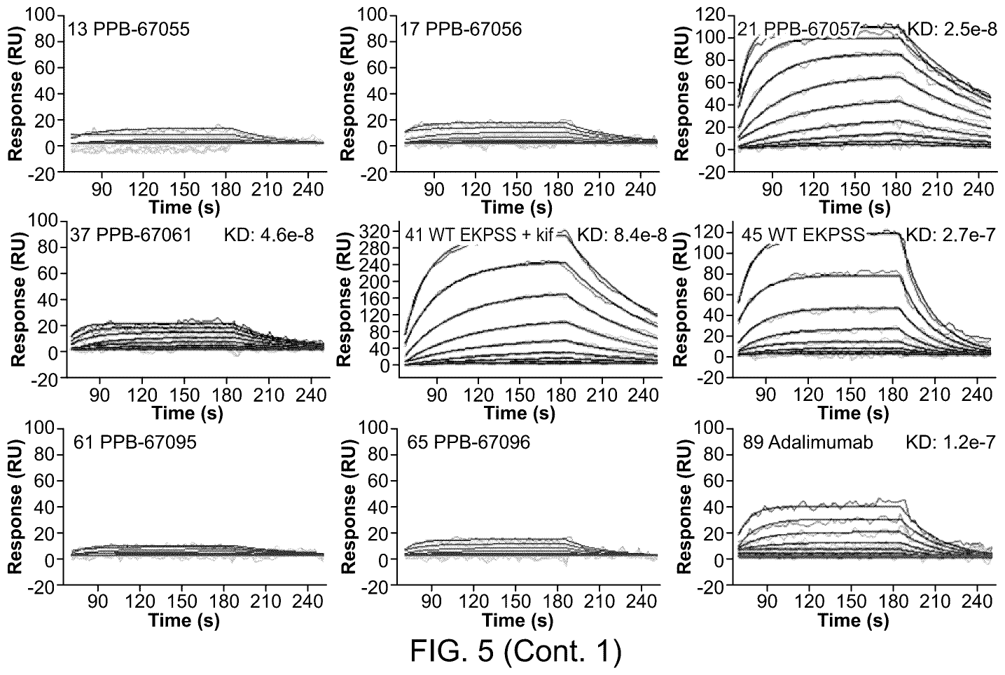

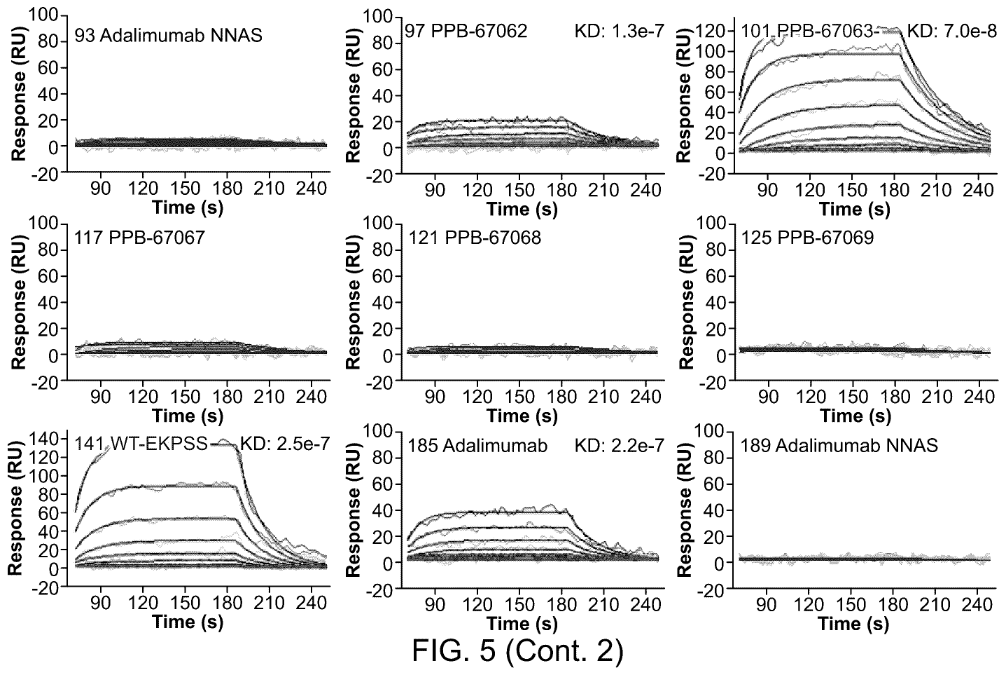

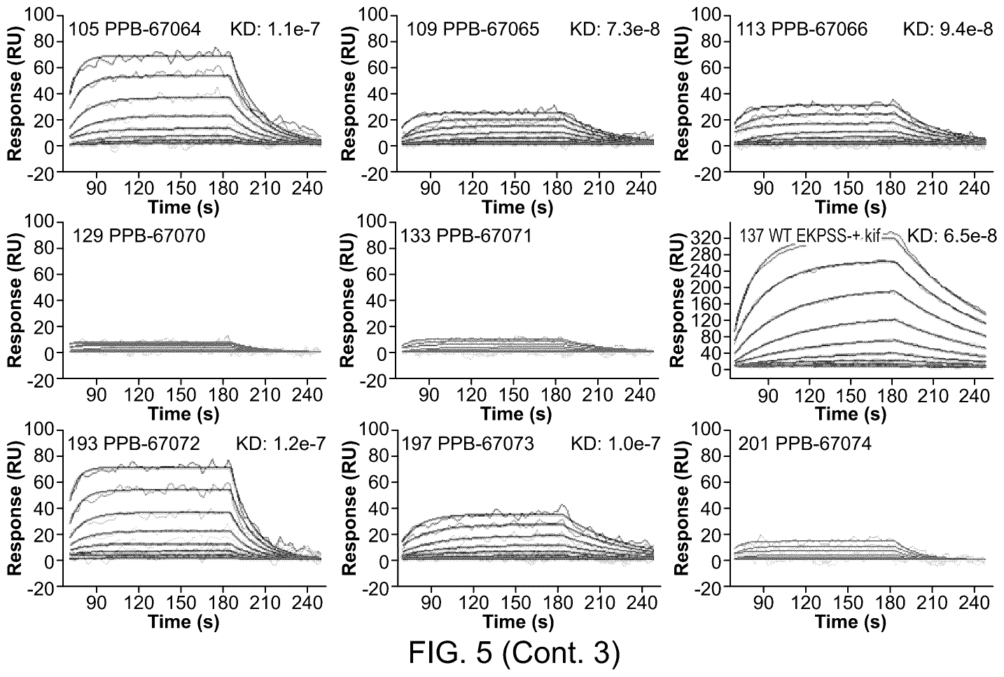

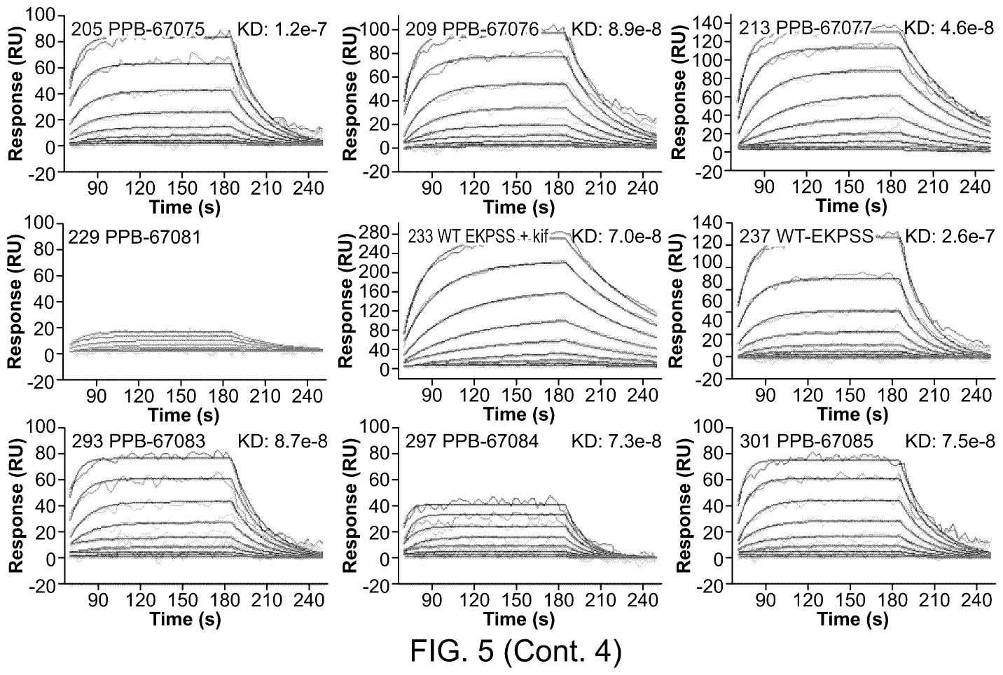

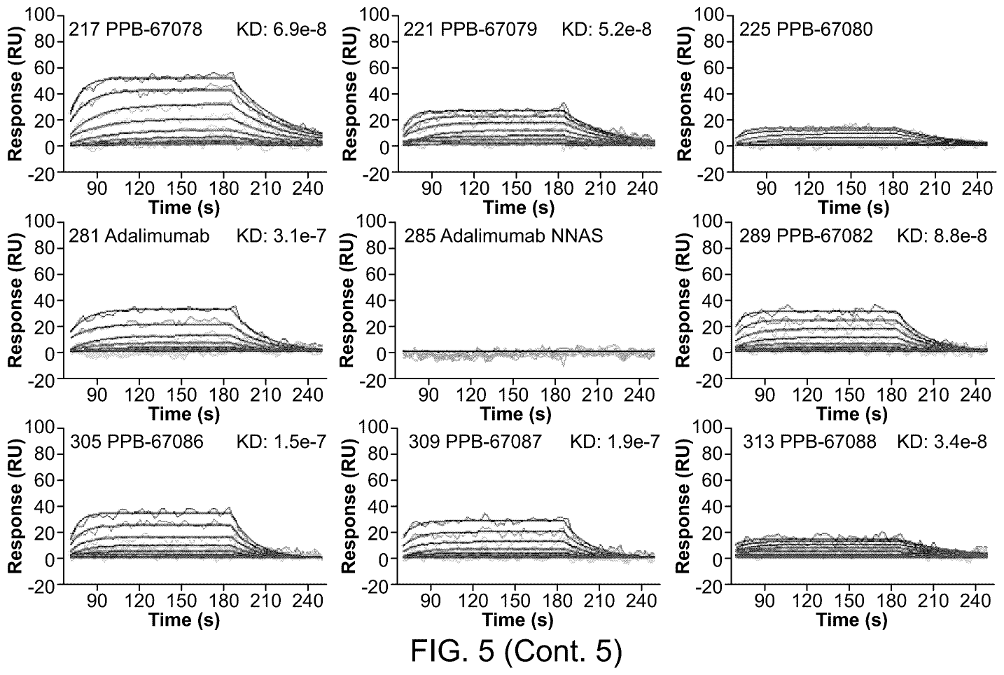

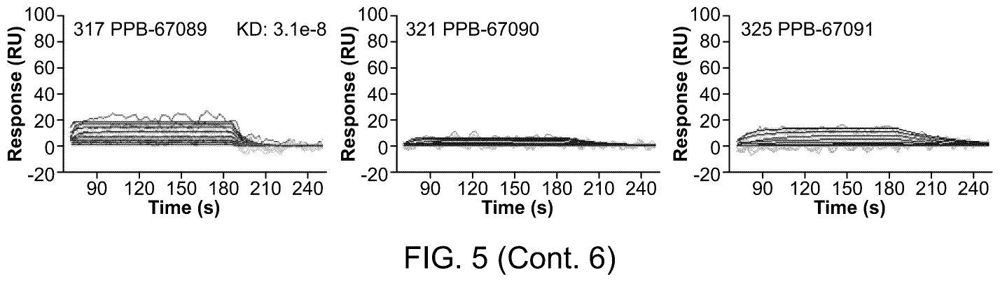

Sensorgram data measuring hFcγRIIIa binding affinity collected for each of the 125 Fc domain variant polypeptides was collected over two plates with respective controls as shown in FIG. 3 and FIG. 4. Example sensorgram data collected from the controls is illustrated in FIG. 5. The controls were: (1) a commercially available antibody with a wild-type IgG domain (adalimumab; left top panel); (2) the same antibody as (1) with a Fc-domain mutation that facilitated glycoengineering which abolished effector functions including hFcγRIIIa binding (an antibody with aS298N/T299A/Y300S Fc domain mutation; right top panel); (3) an antibody with a Fc-domain mutation that enhanced hFcγRIIIa binding (bottom left panel); and (4) the antibody in (3) with glycoengineering that further enhanced hFcγRIIIa binding.

### Conclusion

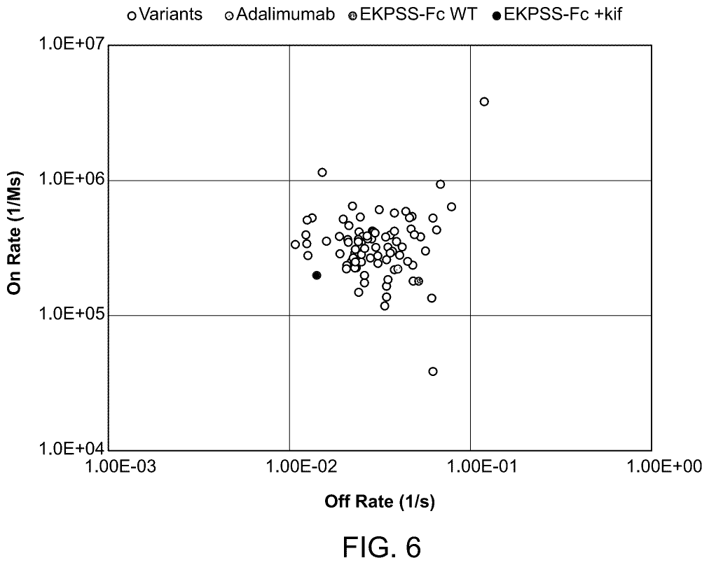

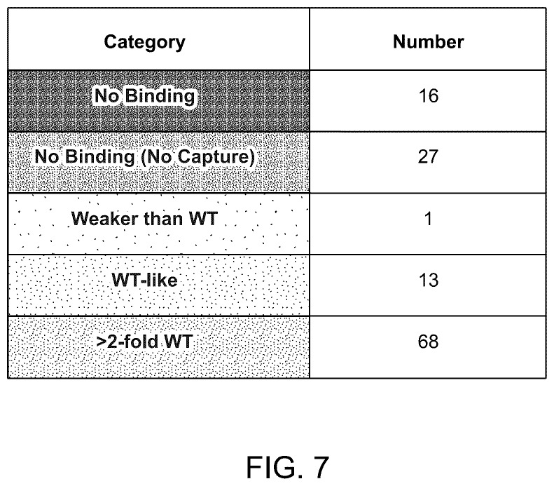

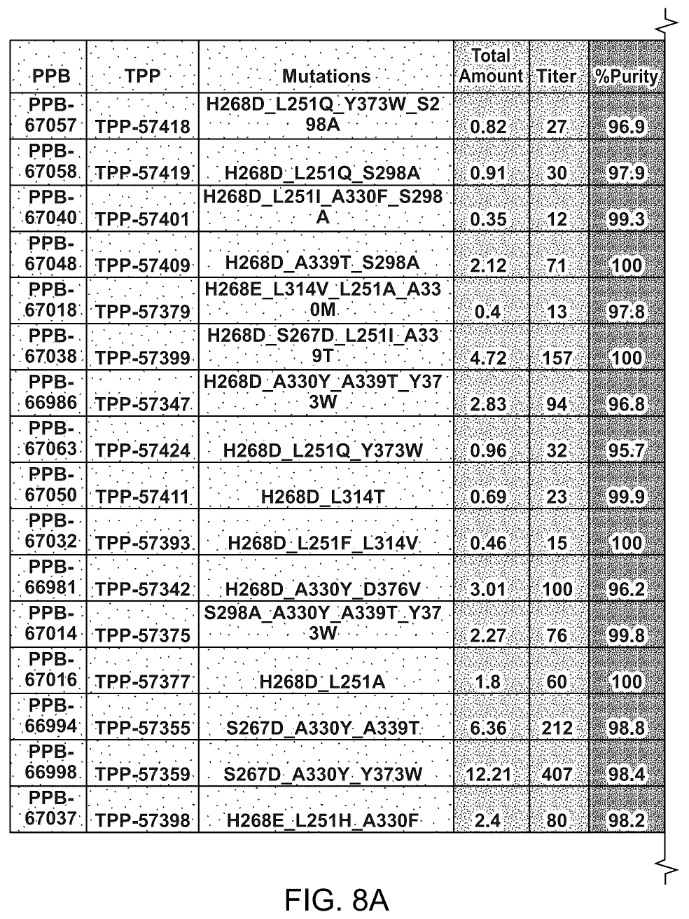

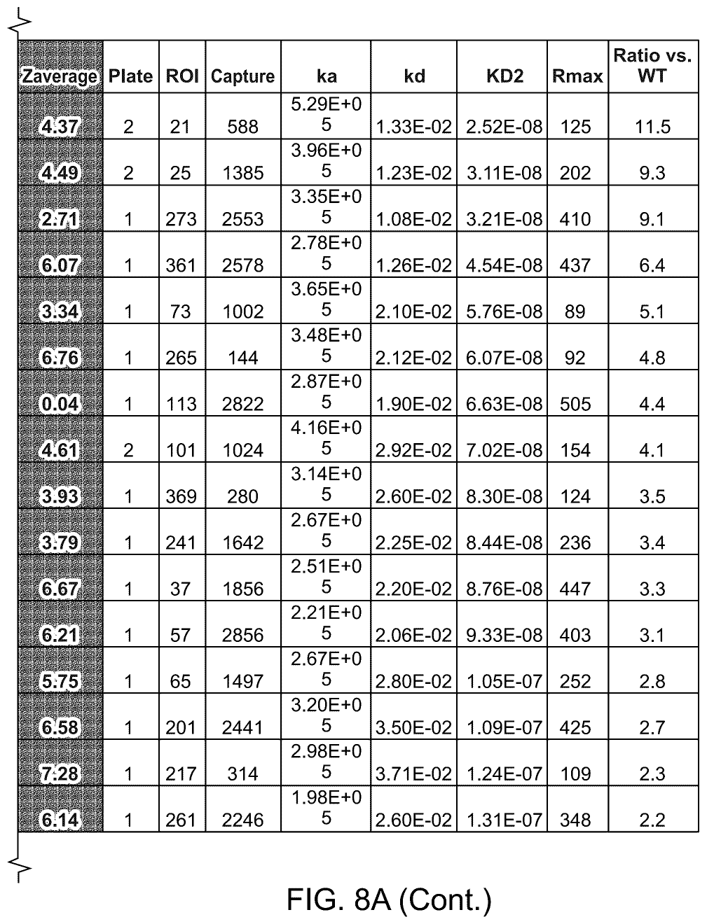

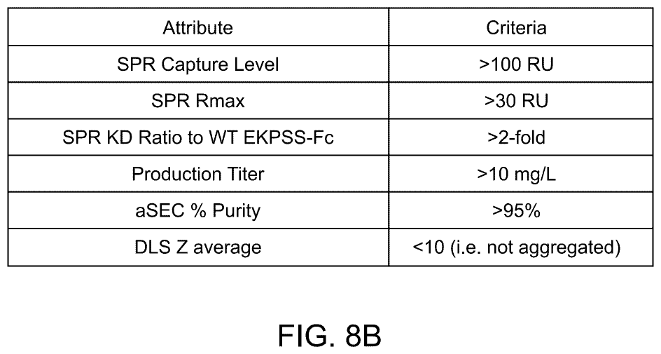

Using sensorgram data from all 125 Fc domain variant polypeptides and controls an isoaffinity dot plot of the Fc domain variant polypeptides hFcγRIIIa binding affinity (Kₐ, 1/Ms, y-axis; K_{d}, 1/s, x-axis) was constructed as shown in FIG. 6. The Fc domain variant polypeptides showed a range of affinities. The Fc domain variant polypeptides were also categorized based on a comparison to the hFcγRIIIa binding affinity of wild-type IgG Fc domain (adalimumab). As shown in FIG. 7, 68 Fc domain variant polypeptides displayed a >2-fold binding affinity relative to a wild-type IgG Fc domain. When the protein expression (exceeding 10 mg/L) and protein purity profile (minimum purity of 95%) of the 68 Fc domain variant polypeptides was applied as assessed as described in Example 1, the number of candidate Fc domain variant polypeptides dropped to 16 Fc domain variant polypeptides. The 16 candidate Fc domain variant polypeptides that met all three criteria (high protein expression, protein purity profile, and binding kinetics) are shown in tabular form in FIG. 8A with FIG. 8B being a legend of the criteria applied. Table 2 provides affinity assessment summary for a series of Fc domain variant polypeptides that were tested.

TABLE 2
FcgRIlla Binding Kinetic Profiles for some Fc domain variant polypeptides

| | | | | Fold- |
|---|---|---|---|---|
| | | | | change |
| | | | | relative |
| Mutations | ka | kd | KD | to WT |
| H268D_L251Q_L314E | 1.15E+06 | 1.52E−02 | 1.33E−08 | 21.9 |
| H268D_L251Q_L314M_S298A | 5.09E+05 | 1.25E−02 | 2.46E−08 | 11.8 |
| H268D_L251Q_Y373W_S298A | 5.29E+05 | 1.33E−02 | 2.52E−08 | 11.5 |
| H268D_L251W_L314S | 3.83E+06 | 1.19E−01 | 3.11E−08 | 9.4 |
| H268D_L251Q_S298A | 3.96E+05 | 1.23E−02 | 3.11E−08 | 9.3 |
| H268D_L251I_A330F_S298A | 3.35E+05 | 1.08E−02 | 3.21E−08 | 9.1 |
| H268D_L251W_L314N | 6.47E+05 | 2.23E−02 | 3.45E−08 | 8.4 |
| H268D_L251F_L314M_S298A | 3.40E+05 | 1.25E−02 | 3.67E−08 | 7.9 |
| H268D_L314W_Y373W_A330M | 5.16E+05 | 1.98E−02 | 3.84E−08 | 7.6 |
| H268E_L251I_L314T_D376V | 3.55E+05 | 1.60E−02 | 4.50E−08 | 6.5 |
| H268D_A339T_S298A | 2.78E+05 | 1.26E−02 | 4.54E−08 | 6.4 |
| H268D_L251Q_L314G | 5.36E+05 | 2.46E−02 | 4.59E−08 | 6.3 |
| H268D_L251W_S298A | 4.63E+05 | 2.13E−02 | 4.60E−08 | 6.3 |
| H268D_A330Y_L314K | 3.85E+05 | 1.89E−02 | 4.91E−08 | 5.9 |
| H268D_L251W_L314T_S298A | 6.07E+05 | 3.13E−02 | 5.17E−08 | 5.6 |
| H268E_L314V_L251A_A330M | 3.65E+05 | 2.10E−02 | 5.76E−08 | 5.1 |
| S298A_A330Y_A339T | 4.15E+05 | 2.42E−02 | 5.84E−08 | 5.0 |
| H268D_S267D_L251I_A339T | 3.48E+05 | 2.12E−02 | 6.07E−08 | 4.8 |
| H268D_L251F_L314K | 3.66E+05 | 2.40E−02 | 6.56E−08 | 4.4 |
| H268D_L251F_L314G | 3.85E+05 | 2.53E−02 | 6.58E−08 | 4.4 |
| H268D_A330Y_L251W_D376V | 5.74E+05 | 3.80E−02 | 6.62E−08 | 4.4 |
| H268D_A330Y_A339T_Y373W | 2.87E+05 | 1.90E−02 | 6.63E−08 | 4.4 |
| A330Y_S267A_D376V_S298A | 4.23E+05 | 2.87E−02 | 6.79E−08 | 4.3 |
| H268D_L251F_L314T | 3.52E+05 | 2.40E−02 | 6.82E−08 | 4.3 |
| H268D_L251W_L314M_S298A | 3.89E+05 | 2.69E−02 | 6.91E−08 | 4.2 |
| H268D_L251Q_Y373W | 4.16E+05 | 2.92E−02 | 7.02E−08 | 4.1 |
| WT EKPSS + kif | 1.99E+05 | 1.40E−02 | 7.08E−08 | 4.1 |
| H268D_A330Y_Y373W | 3.49E+05 | 2.50E−02 | 7.17E−08 | 4.1 |
| H268D_L251Q_L314M_Y373W | 4.08E+05 | 2.97E−02 | 7.28E−08 | 4.0 |
| H268D_L251W | 9.36E+05 | 6.82E−02 | 7.28E−08 | 4.0 |
| H268D_L251F_L314S | 3.70E+05 | 2.70E−02 | 7.30E−08 | 4.0 |
| H268D_L251F | 3.28E+05 | 2.43E−02 | 7.39E−08 | 3.9 |
| H268D_L251W_A330Y_Y373W | 5.90E+05 | 4.40E−02 | 7.46E−08 | 3.9 |
| H268D_L251F_L314N | 3.09E+05 | 2.31E−02 | 7.49E−08 | 3.9 |
| S298A_A330Y_D376V | 3.69E+05 | 2.85E−02 | 7.72E−08 | 3.8 |
| H268D_L251F_L314M_Y373W | 2.89E+05 | 2.30E−02 | 7.95E−08 | 3.7 |
| H268D_L314T | 3.14E+05 | 2.60E−02 | 8.30E−08 | 3.5 |
| H268D_L251F_L314V | 2.67E+05 | 2.25E−02 | 8.44E−08 | 3.4 |
| H268D_L251W_Y373W | 5.30E+05 | 4.60E−02 | 8.68E−08 | 3.4 |
| H268D_A330Y_D376V | 2.51E+05 | 2.20E−02 | 8.76E−08 | 3.3 |
| H268D_L251W_L314K | 5.42E+05 | 4.76E−02 | 8.78E−08 | 3.3 |
| H268D_L251F_Y373W | 2.58E+05 | 2.28E−02 | 8.82E−08 | 3.3 |
| H268D_L251F_L314M | 2.83E+05 | 2.50E−02 | 8.83E−08 | 3.3 |
| S298A_A330Y_L314K | 2.35E+05 | 2.07E−02 | 8.84E−08 | 3.3 |
| H268D_L251W_Y373W_S298A | 3.81E+05 | 3.40E−02 | 8.91E−08 | 3.3 |
| S298A_A330Y_L251W | 4.21E+05 | 3.80E−02 | 9.04E−08 | 3.2 |
| S267D_A330Y_L251W_D376V | 3.94E+05 | 3.60E−02 | 9.14E−08 | 3.2 |
| H268D_L251W_L314V | 2.49E+05 | 2.31E−02 | 9.28E−08 | 3.1 |
| S298A_A330Y_A339T_Y373W | 2.21E+05 | 2.06E−02 | 9.33E−08 | 3.1 |
| H268D_L251Q_L314M | 3.19E+05 | 3.00E−02 | 9.39E−08 | 3.1 |
| H268D_L314M_L251A_Y373W | 2.49E+05 | 2.50E−02 | 1.00E−07 | 2.9 |
| H268E_L314W_L251Q_A330Y | 2.25E+05 | 2.30E−02 | 1.02E−07 | 2.8 |
| L251F | 2.26E+05 | 2.34E−02 | 1.04E−07 | 2.8 |
| H268D_L251A | 2.67E+05 | 2.80E−02 | 1.05E−07 | 2.8 |
| H268D_L251Q | 4.38E+05 | 4.70E−02 | 1.07E−07 | 2.7 |
| S267D_A330Y_A339T | 3.20E+05 | 3.50E−02 | 1.09E−07 | 2.7 |
| S267D_A330Y_L251W | 3.53E+05 | 3.91E−02 | 1.11E−07 | 2.6 |
| H268E_L251W_L314I_A330F | 2.76E+05 | 3.08E−02 | 1.11E−07 | 2.6 |
| H268E_L251Q_A339T_L314A | 5.26E+05 | 6.20E−02 | 1.18E−07 | 2.5 |
| A330Y_D376V_Y373W | 6.37E+05 | 7.86E−02 | 1.23E−07 | 2.4 |
| H268D_L251W_S267D_Y373W | 3.97E+05 | 4.90E−02 | 1.23E−07 | 2.4 |
| S267D_L314W_L251H_D376V | 2.90E+05 | 3.60E−02 | 1.24E−07 | 2.3 |
| S267D_A330Y_Y373W | 2.98E+05 | 3.71E−02 | 1.24E−07 | 2.3 |
| S298A_A330Y_L251W_D376V | 2.43E+05 | 3.08E−02 | 1.27E−07 | 2.3 |
| S267D_A330Y_A339T_Y373W | 3.22E+05 | 4.20E−02 | 1.31E−07 | 2.2 |
| H268E_L251H_A330F | 1.98E+05 | 2.60E−02 | 1.31E−07 | 2.2 |
| H268D_L314K_L251Q | 2.59E+05 | 3.44E−02 | 1.33E−07 | 2.2 |
| A330Y_L314A_D376N | 3.82E+05 | 5.30E−02 | 1.39E−07 | 2.1 |
| S267D_A330Y_D376V | 2.79E+05 | 4.06E−02 | 1.45E−07 | 2.0 |
| H268D_A330Y_L251W | 1.74E+05 | 2.60E−02 | 1.49E−07 | 2.0 |
| H268D_L251W_L314M_Y373W | 4.31E+05 | 6.50E−02 | 1.51E−07 | 1.9 |
| H268D_L251F_L314W | 1.48E+05 | 2.41E−02 | 1.63E−07 | 1.8 |
| L251F_S267A_L314S | 2.18E+05 | 3.80E−02 | 1.74E−07 | 1.7 |
| L314W_A339T_A330M_D376T | 2.52E+05 | 4.50E−02 | 1.79E−07 | 1.6 |
| H268D_L251W_L314M | 3.00E+05 | 5.64E−02 | 1.88E−07 | 1.5 |
| L314V_A330F_Y373W_H268A | 1.84E+05 | 3.50E−02 | 1.90E−07 | 1.5 |
| L251F_A339T_L314S_Y373W | 2.35E+05 | 4.80E−02 | 2.04E−07 | 1.4 |
| S267D_L314T_A339T | 1.65E+05 | 3.44E−02 | 2.08E−07 | 1.4 |
| S298A_A330Y_Y373W | 1.37E+05 | 3.44E−02 | 2.51E−07 | 1.2 |
| S267D_A330Y_L314K | 1.80E+05 | 4.82E−02 | 2.68E−07 | 1.1 |
| L314N_S267A_A330M_D376T | 1.17E+05 | 3.36E−02 | 2.86E−07 | 1.0 |
| WT | 1.82E+05 | 5.18E−02 | 2.89E−07 | 1.0 |
| L251I_L314G_A339T_A330F | 3.85E+04 | 6.20E−02 | 1.61E−06 | 0.2 |

In a further study, thermal stability assessments were carried out determine the melting temperatures for a few of the Fc domain variant polypeptides from Table 2, comprising a mutation at position L251.

TABLE 3
Thermal stability of a few Fc domain variant polypeptides
comprising a mutation at position L251

| | Tm | Tonset |
|---|---|---|
| Fc domain variant | (° C.) | (° C.) |
| H268D, L251I, A330F, S298A | 53.5 | 47.4 |
| H268D, L251Q, S298A | 44.8 | |
| H268D, L251Q, Y373W, S298A | 44.9 | |
| H268D, L251Q, Y373W | 46.8 | 42.1 |
| H268D, L251I, S267D, A339T | 46.2 | 41.6 |
| H268D, L251Q, L314M, S298A | 44.8 | |
| H268D, L251Q, Y373W, S298A | 46.5 | 37.9 |
| H268D, L251Q, S298A | 46.3 | 41.3 |
| H268D, L251F, L314V | 52.5 | 47.5 |
| H268D, A330Y, D376V | 54.2 | 48 |
| H268D, L251Q, L314M | 51.9 | 46.8 |
| H268E, L314V, L251A, A330M | 47.9 | 41.6 |
| WT | 67.3 | 60.6 |

In order to further explore the effect of mutation at position L251, additional Fc domain variants were tested, with or without mutations at position L251.

As shown in Table 4, all tested Fc domain variant polypeptides displayed enhanced binding affinity relative to a wild-type IgG Fc domain (WT). It was observed that inclusion of a mutation at position L251 did enhance the FcγRIIIa binding (e.g., while H268D, L251Q, S298A combination exhibited around 8.7-fold enhancement in binding affinity compared to the WT, reversion of the mutation at position L251, as seen in the combination of H268D, S298A decreased the fold enhancement relative to WT to about 5-fold). Similar effect was observed when comparing the combinations: (A) H268D, L251Q, Y373W, S298A (about 10.6 fold enhancement) compared to H268D, Y373W, S298A (about 5.8-fold enhancement); and (B) H268D, L251Q, L314M, S298A (about 7.8-fold enhancement) compared to H268D, L314M, S298A (about 5.6-fold enhancement) relative to WT.

In terms of thermal stability however, the variants comprising the wild-type residue in position 251 (L251) had higher melting temperatures (Tm, in 00) when compared to the corresponding combinations with L251X mutations. Results are summarized in Table 4. See, e.g., comparison of H268D, L251Q, Y373W, S298A combination with a Tm of 44.9° C. and H268D, Y373W, S298A combination with a Tm of 62° C.

TABLE 4
Summary table for Fc domain variant polypeptides

| Mutations | ka | kd | KD | SD | Fold Change | Tm | Tonset |
|---|---|---|---|---|---|---|---|
| (if Applicable) | (1/Ms) | (1/s) | (M) | (M) | vs. WT | (° C.) | (° C.) |
| H268D, L251Q, | 4.78E+05 | 2.15E−02 | 4.50E−08 | 2.91E−09 | 10.6 | 44.9 | 38.7 |
| Y373W, S298A | | | | | | | |
| H268D, L314T, | 4.65E+05 | 2.17E−02 | 4.68E−08 | 3.13E−09 | 10.2 | 53.8 | 48.4 |
| S298A | | | | | | | |
| H268D, L251Q, | 4.73E+05 | 2.59E−02 | 5.50E−08 | 3.68E−09 | 8.7 | 44.8 | 38.7 |
| S298A | | | | | | | |
| H268D, L251Q, | 5.09E+05 | 2.79E−02 | 6.08E−08 | 2.36E−08 | 7.8 | 44.8 | 40.6 |
| L314M, S298A | | | | | | | |
| H268D, A330F, | 3.89E+05 | 3.11E−02 | 8.01E−08 | 2.21E−09 | 6.0 | 62.5 | 56.3 |
| S298A | | | | | | | |
| H268D, Y373W, | 3.32E+05 | 2.73E−02 | 8.26E−08 | 6.23E−09 | 5.8 | 62 | 55.7 |
| S298A | | | | | | | |
| H268D, L314M, | 3.59E+05 | 3.05E−02 | 8.51E−08 | 3.92E−09 | 5.6 | 59.1 | 53.2 |
| S298A | | | | | | | |
| H268D, S298A | 3.28E+05 | 3.14E−02 | 9.57E−08 | 4.06E−09 | 5.0 | 62.3 | 56.1 |
| H268D, L314N | 4.71E+05 | 4.52E−02 | 9.65E−08 | 5.78E−09 | 4.9 | 56.9 | 50.6 |
| H268D, S267D, | 4.42E+05 | 5.25E−02 | 1.19E−07 | 9.23E−09 | 4.0 | 56.9 | 51.2 |
| A339T | | | | | | | |
| H268D, L314T | 3.84E+05 | 4.81E−02 | 1.25E−07 | 6.73E−09 | 3.8 | 54.4 | 48.3 |
| H268D, L314E | 4.16E+05 | 5.76E−02 | 1.40E−07 | 1.68E−08 | 3.4 | 56.6 | 50.3 |
| H268D, L314G | 3.19E+05 | 4.66E−02 | 1.46E−07 | 6.95E−09 | 3.3 | 51.9 | 46.5 |
| H268D, L314S | 2.86E+05 | 4.34E−02 | 1.52E−07 | 7.92E−09 | 3.1 | 54.3 | 48.6 |
| H268D, A330Y, | 2.67E+05 | 4.06E−02 | 1.52E−07 | 9.94E−09 | 3.1 | 52.8 | 47.9 |
| D376V | | | | | | | |
| H268E, L314T, | 4.04E+05 | 6.40E−02 | 1.59E−07 | 4.95E−09 | 3.0 | 48.9 | 42.9 |
| D376V | | | | | | | |
| H268D, L314K | 3.24E+05 | 5.16E−02 | 1.59E−07 | 9.53E−09 | 3.0 | 53.8 | 48.3 |
| H268E, L314V, | 2.06E+05 | 3.29E−02 | 1.60E−07 | 6.78E−09 | 3.0 | 56.5 | 50.2 |
| A330M | | | | | | | |
| H268D, Y373W | 2.90E+05 | 6.38E−02 | 2.21E−07 | 1.82E−08 | 2.2 | 62.7 | 55.9 |
| WT | 2.18E+05 | 9.98E−02 | 4.77E−07 | 6.36E−08 | 1.0 | 67.3 | 60.6 |

1. A Fc domain variant polypeptide, wherein the Fc domain variant polypeptide comprises at least two substitutions at amino acid positions 251, 267, 268, 298, 314, 330, 339, 373, and 376, according to EU numbering, as compared to a Fc domain parent polypeptide.

2. The Fc domain variant polypeptide of claim 1, wherein the Fc domain variant polypeptide has increased binding to a Fc receptor relative to the Fc domain parent polypeptide.

3. The Fc domain variant polypeptide of claim 1, wherein the at least two amino acid substitutions are selected from:
(i) an alanine (A), a histidine (H), an isoleucine (I), a phenylalanine (F), a glutamine (Q), or a tryptophan (W) at amino acid position 251,
(ii) an alanine (A) or an aspartic acid (D) at amino acid position 267,
(iii) an aspartic acid (D) or a glutamic acid (E) at amino acid position 268,
(iv) an alanine (A) at amino acid position 298,
(v) a glycine (G), a lysine (K), an asparagine (N), a methionine (M), a serine (S), a threonine (T), a valine (V), a glutamic acid (E), or a tryptophan (W) at amino acid position 314,
(vi) a phenylalanine (F), a methionine (M), or a tyrosine (Y) at amino acid position 330,
(vii) a threonine (T) at amino acid position 339,
(viii) a tryptophan (W) at amino acid position 373, or
(ix) a valine (V) at amino acid position 376,
according to EU numbering.

4. The Fc domain variant polypeptide of claim 1, wherein the Fc domain parent polypeptide is a wild-type Fc domain.

5. The Fc domain variant polypeptide of claim 1, wherein the Fc domain variant comprises:
an aspartic acid (D) at amino acid position 268, a glutamine (Q) at amino acid position 251, a tryptophan (W) at amino acid position 373, and an alanine (A) at amino acid position 298;
an aspartic acid (D) at amino acid position 268, a glutamine (Q) at amino acid position 251, and an alanine (A) at amino acid position 298;
an aspartic acid (D) at amino acid position 268, an isoleucine (I) at amino acid position 251, a phenylalanine (F) at amino acid position 330, and an alanine (A) at amino acid position 298;
an aspartic acid (D) at amino acid position 268, a threonine (T) at amino acid position 339, and an alanine (A) at amino acid position 298;
a glutamic acid (E) at amino acid position 268, a valine (V) at amino acid position 376, an alanine (A) at amino acid position 251, and a methionine (M) at amino acid position 330;
an aspartic acid (D) at amino acid position 268, an aspartic acid (D) at amino acid position 267, an isoleucine (I) at amino acid position 251, and a threonine (T) at amino acid position 339;
an aspartic acid (D) at amino acid position 268, a tyrosine (Y) at amino acid position 330, a threonine (T) at amino acid position 339, a tryptophan (W) at amino acid position 373;
an aspartic acid (D) at amino acid position 268, a glutamine (Q) at amino acid position 251, a tryptophan (W) at amino acid position 373;
an aspartic acid (D) at amino acid position 268 and a threonine (T) at amino acid position 314; an aspartic acid (D) at amino acid position 268, a phenylalanine (F) at amino acid position 251, and a valine (V) at amino acid position 314;
an aspartic acid (D) at amino acid position 268, a tyrosine (Y) at amino acid position 330, a valine (V) at amino acid position 376;
an alanine (A) at amino acid position 298, a tyrosine (Y) at amino acid position 330, a threonine (T) at amino acid position 339, and a tryptophan (W) at amino acid position 373;
an aspartic acid (D) at amino acid position 268 and an alanine (A) at amino acid position 251,
an aspartic acid (D) at amino acid position 267, a tyrosine (Y) at amino acid position 330, a threonine (T) at amino acid position 339;
an aspartic acid (D) at amino acid position 267, a tyrosine (Y) at amino acid position 330, a tryptophan (W) at amino acid position 373;
a glutamic acid (E) at amino acid position 268, a histidine (H) at amino acid position 251, a phenylalanine (F) at amino acid position 330;
an aspartic acid (D) at amino acid position 268, a tryptophan (W) at amino acid position 373, an alanine (A) at amino acid position 298;
an aspartic acid (D) at amino acid position 268 and an alanine (A) at amino acid position 298; an aspartic acid (D) at amino acid position 268, a phenylalanine (F) at amino acid position 330, and an alanine (A) at amino acid position 298;
a glutamic acid (E) at amino acid position 268, a valine (V) at amino acid position 314, a methionine (M) at amino acid position 330;
an aspartic acid (D) at amino acid position 268, an aspartic acid (D) at amino acid position 267, a threonine (T) at amino acid position 339;
an aspartic acid (D) at amino acid position 268 and a tryptophan (W) at amino acid position 373;
an aspartic acid (D) at amino acid position 268 and a valine (V) at amino acid position 314;
a glutamic acid (E) at amino acid position 268 and a phenylalanine (F) at amino acid position 330;
an aspartic acid (D) at amino acid position 268, a glutamine (Q) at amino acid position 251, and a glutamic acid (E) at amino acid position 314;
an aspartic acid (D) at amino acid position 268, a glutamine (Q) at amino acid position 251, and a methionine (M) at amino acid position 314, and an alanine (A) at amino acid position 298;
an aspartic acid (D) at amino acid position 268, a tryptophan (W) at amino acid position 251, and a serine (S) at amino acid position 314;
an aspartic acid (D) at amino acid position 268, a tryptophan (W) at amino acid position 251, and an asparagine (N) at amino acid position 314;
an aspartic acid (D) at amino acid position 268, a phenylalanine (F) at amino acid position 251, a methionine (M) at amino acid position 314, and an alanine (A) at amino acid position 298;
an aspartic acid (D) at amino acid position 268, a tryptophan (W) at amino acid position 314, a tryptophan (W) at amino acid position 373, and a methionine (M) at amino acid position 330;
a glutamic acid (E) at amino acid position 268, an isoleucine (I) at amino acid position 251, a threonine (T) at amino acid position 314, and a valine (V) at amino acid position 376;
an aspartic acid (D) at amino acid position 268, a glutamine (Q) at amino acid position 251, and a glycine (G) at amino acid position 314;
an aspartic acid (D) at amino acid position 268, a tryptophan (W) at amino acid position 251, and a glycine (G) at amino acid position 314, and an alanine (A) at amino acid position 298;
an aspartic acid (D) at amino acid position 268, a tyrosine (Y) at amino acid position 330, and a lysine (K) at amino acid position 314;
an aspartic acid (D) at amino acid position 268, a tryptophan (W) at amino acid position 251, a threonine (T) at amino acid position 314, and an alanine (A) at amino acid position 298;
a glutamic acid (E) at amino acid position 268, a valine (V) at amino acid position 314, an alanine (A) at amino acid position 251, and a methionine (M) at amino acid position 330;
an alanine (A) at amino acid position 298, a tyrosine (Y) at amino acid position 330, and a threonine (T) at amino acid position 339;
an aspartic acid (D) at amino acid position 268, a phenylalanine (F) at amino acid position 251, and a lysine (K) at amino acid position 314;
an aspartic acid (D) at amino acid position 268, a phenylalanine (F) at amino acid position 251, and a glycine (G) at amino acid position 314;
an aspartic acid (D) at amino acid position 268, a tyrosine (Y) at amino acid position 330, a tryptophan (W) at amino acid position 251, and a valine (V) at amino acid position 376;
a tyrosine (Y) at amino acid position 330, an alanine (A) at amino acid position 267, a valine (V) at amino acid position 376, and an alanine (A) at amino acid position 298;
an aspartic acid (D) at amino acid position 268, a phenylalanine (F) at amino acid position 251, and a threonine (T) at amino acid position 314;
an aspartic acid (D) at amino acid position 268, a tryptophan (W) at amino acid position 251, a methionine (M) at amino acid position 314, and an alanine (A) at amino acid position 298;
an aspartic acid (D) at amino acid position 268, a tyrosine (Y) at amino acid position 330, and a tryptophan (W) at amino acid position 373;
an aspartic acid (D) at amino acid position 268 and a glutamic acid (E) at amino acid position 314;
an aspartic acid (D) at amino acid position 268, a methionine (M) at amino acid position 314, and an alanine (A) at amino acid position 298;
an aspartic acid (D) at amino acid position 268, a tryptophan (W) at amino acid position 373, and an alanine (A) at amino acid position 298;
an aspartic acid (D) at amino acid position 268 and a serine (S) at amino acid position 314;
an aspartic acid (D) at amino acid position 268 and an asparagine (N) at amino acid position 314,
a glutamic acid (E) at amino acid position 268, a threonine (T) at amino acid position 314, and a valine (V) at amino acid position 376;
an aspartic acid (D) at amino acid position 268 and a glycine (G) at amino acid position 314,
an aspartic acid (D) at amino acid position 268, a threonine (T) at amino acid position 314, and an alanine (A) at amino acid position 298;
a glutamic acid (E) at amino acid position 268, a valine (V) at amino acid position 314, and a methionine (M) at amino acid position 330;
an aspartic acid (D) at amino acid position 268 and a lysine (K) at amino acid position 314;
an aspartic acid (D) at amino acid position 268, a tyrosine (Y) at amino acid position 330, and a valine (V) at amino acid position 376;
an aspartic acid (D) at amino acid position 268, a glutamine (Q) at amino acid position 251, a methionine (M) at amino acid position 314, and an alanine (A) at amino acid position 298;
an aspartic acid (D) at amino acid position 268, an aspartic acid (D) at amino acid position 267, and a threonine (T) at amino acid position 339;
an aspartic acid (D) at amino acid position 268 and a threonine (T) at position 314;
an aspartic acid (D) at amino acid position 268 and a glutamic acid (E) at position 314;
an aspartic acid (D) at amino acid position 268 and a glycine (G) at position 314;
an aspartic acid (D) at amino acid position 268 and a serine (S) at position 314;
an aspartic acid (D) at amino acid position 268, a tyrosine (Y) at amino acid position 330, a valine (V) at position 376;
a glutamic acid (E) at amino acid position 268, a threonine (T) at amino acid position 314, a valine (V) at position 376: or
an aspartic acid (E) at amino acid position 268, a valine (V) at amino acid position 314, and a methionine (M) at amino acid position 330,
according to EU numbering.

6-68. (canceled)

69. The Fc domain variant polypeptide of claim 1, wherein the Fc domain variant is of mammalian origin, optionally wherein
the Fc domain variant is of human origin,
the Fc domain variant is derived from an immunoglobulin class selected from a group consisting of: IgM, IgG, IgD, IgA, and IgE,
the Fc domain variant is derived from an IgG Fc domain,
the Fc domain variant is derived from a IgG1 Fc domain or an IgG4 Fc domain, or the Fc domain variant is about 20%, 30%, 40%, 50%, 60%, 70%, 90% or more afucosylated.

70-73. (canceled)

74. The Fc domain variant polypeptide of claim 1, further comprising
an aspartic acid (D) at amino acid position 239,
a glutamic acid (E) at amino acid position 332,
an aspartic acid (D) at amino acid position 239 and a glutamic acid (E) at amino acid position 332, or
a cysteine (C) at amino acid position 292 and a cysteine (C) at amino acid position 302.

75-78. (canceled)

79. The Fc domain variant polypeptide of claim 1, wherein the Fc domain variant comprises at least one N-glycan, wherein the N-glycan comprises a mannose and/or a GlcNAc.

80. (canceled)

81. The Fc domain variant polypeptide of claim 79, wherein:
the N-glycan is an oligomannose-type, optionally wherein
the oligomannose-type N-glycan comprise an oligosaccharide selected from the group consisting of Man₉(GlcNAc)₂, Man₈(GlcNAc)₂, Man₇(GlcNAc)₂, Man₆(GlcNAc)₂, and Man₅(GlcNAc)₂;
the Fc domain variant polypeptide comprises 20%, 30%, 40%, 50%, 60%, 70%, 90% or more Man₅₋₉(GlcNAc)₂ glycans by molar ratio relative to all N-glycans;
the Fc domain variant polypeptide comprises greater than 70%, 75%, 80%, 85%, 90%, or 95% Man₅₋₉(GlcNAc)₂N-glycans by molar ratio relative to all N-glycans;
the Fc domain variant polypeptide comprises Man₈ and Man₉ together as the major species of Man₅₋₉(GlcNAc)₂ N-glycans;
the Fc domain variant polypeptide comprises at least 97% Man₅₋₉(GlcNAc)₂N-glycans by molar ratio relative to all N-glycans;
the Fc domain variant polypeptide comprises Man₈ and Man₉ together as the major species of Man₅₋₉(GlcNAc)₂ N-glycans;
the Fc domain variant polypeptide comprises at least 97% Man₅₋₉(GlcNAc)₂N-glycans by molar ratio relative to all N-glycans: or
the Fc domain variant polypeptide comprises less than 30%, 20%, 10%, 5%, 1% of Man₅₋₉(GlcNAc)₂ glycans by molar ratio relative to all N-glycans, or substantially no Man₅₋₉(GlcNAc)₂ glycans by molar ratio relative to all N-glycans.

82-87. (canceled)

88. The Fc domain variant polypeptide of claim 79, wherein:
the Fc domain variant polypeptide further comprises a cysteine (C) at amino acid position 292 and a cysteine (C) at amino acid position 302,
the Fc domain variant polypeptide is produced by culturing cells that express the Fc domain variant polypeptide in the presence of a mannosidase inhibitor;
the mannosidase inhibitor is kifunensine;
the concentration of kifunensine is from about 60 ng/mL to about 2500 ng/mL: or
the concentration of kifunensine is about 2000 ng/mL.

89-92. (canceled)

93. A binding polypeptide comprising at least one binding site for selectively binding to a target antigen of interest and the Fc domain variant polypeptide of claim 1.

94. The binding polypeptide of claim 93, wherein the binding polypeptide is
a multispecific binding polypeptide,
an antibody or an antigen fragment thereof;
a multispecific antibody; and/or
wherein the binding polypeptide comprises a VHH,
wherein the VHH comprises at least one antigen binding fragment selected from a group consisting of: a variable fragment (Fv), a Fab, a Fab′, a F(ab′)₂, a minibody, a diabody, a triabody, a tetrabody, a tandem di-scFv, a tandem tri-scFv, an immunoglobulin single variable domain (ISV).

95-98. (canceled)

99. The Fc domain variant polypeptide claim 1, wherein the Fc receptor is a human Fc receptor;
the Fc receptor is a Fcγ receptor (FcγR);
the FcγR is a FcγRI, FcγRIIA, FcγRIIB, FcγRIIIA, or a FcγRIIIB;
the FcγRIIIa is hFcγRIIIa with a valine at amino acid position 158 (FcγRIIIav₁₅₈), according to EU numbering;
the FcγRIIIa is hFcγRIIIa with a phenylalanine at amino acid position 158 (FcγRIIIa_{F158}), according to EU numbering; or
the Fc receptor is a neonatal Fc receptor (FcRn).

100-104. (canceled)

105. The Fc domain variant polypeptide of claim 1, wherein the Fc domain parent polypeptide is a wild-type Fc domain or a modified Fc domain.

106. (canceled)

107. The Fc domain variant polypeptide of claim 105, wherein the Fc domain parent polypeptide comprises at least one modified glycan, optionally wherein
the modified glycan is a bis mannose 6 phosphate (bisM6P), a disaccharide mannose 6 phosphate, or a mannose 6 phosphate monosaccharide, and further wherein
the modified glycan is an oligomannose-type N-glycan comprising an oligosaccharide selected from the group consisting of: a Man₉(GlcNAc)₂, a Man₈(GlcNAc)₂, a Man₇(GlcNAc)₂, a Man₆(GlcNAc)₂, or a Man₅(GlcNAc)₂.

108-109. (canceled)

110. The Fc domain variant polypeptide of claim 1, wherein:
the Fc domain variant polypeptide has about a 1.5-fold to a 20-fold increased binding affinity to a Fc receptor relative to the Fc domain parent polypeptide,
the polypeptide comprising the Fc domain variant has production titer ranges from about 5 to about 500 milligrams per liter (mg/L)
the polypeptide comprising the Fc domain variant has at least 60, 70, 80, 90, or 100% purity when measured by size exclusion chromatography (SEC): or
the polypeptide comprising the Fc domain variant exhibits a melting temperature (Tm) of greater than about 44° C.

111-113. (canceled)

114. A pharmaceutical composition comprising a Fc domain variant polypeptide wherein the Fc domain variant polypeptide comprises at least two substitutions at amino acid positions 251, 267, 268, 298, 314, 330, 339, 373, and 376, according to EU numbering, as compared to a Fc domain parent polypeptide and a pharmaceutically acceptable carrier or diluent.

115. A nucleic acid molecule encoding a Fc domain variant polypeptide wherein the Fc domain variant polypeptide comprises at least two substitutions at amino acid positions 251, 267, 268, 298, 314, 330, 339, 373, and 376, according to EU numbering, as compared to a Fc domain parent polypeptide.

116. The nucleic acid molecule of claim 115, wherein a vector comprises the nucleic acid molecule encoding the Fc domain variant polypeptide.

117. A producing cell that expresses the nucleic acid molecule according to claim 115.
